(19) United States
(12) Patent Application Publication
(10) Pub. No.: US 2024/0076288 A1
(43) Pub. Date: Mar. 7, 2024

(54) HETEROCYCLIC COMPOUND AND ORGANIC LIGHT-EMITTING DEVICE INCLUDING THE SAME

(71) Applicants: Samsung Electronics Co., Ltd., Suwon-si (KR); Samsung SDI Co., Ltd., Yongin-si (KR)

(72) Inventors: Minsik MIN, Suwon-si (KR); Eunsuk Kwon, Suwon-si (KR); Kyungdoc Kim, Seoul (KR); Inkoo KIM, Suwon-si (KR); Juhyun KIM, Suwon-si (KR); Joonghyuk KIM, Seoul (KR); Jhunmo SON, Yongin-si (KR); Yongsik JUNG, Seoul (KR)

(21) Appl. No.: 18/380,344

(22) Filed: Oct. 16, 2023

Related U.S. Application Data
(63) Continuation of application No. 16/716,966, filed on Dec. 17, 2019.

(30) Foreign Application Priority Data
Dec. 21, 2018 (KR) 10-2018-0167893

(51) Int. Cl. C07D 405/10; C07D 403/10; C07D 409/10; H10K 85/60

(52) U.S. Cl. CPC C07D 405/10; C07D 403/10; C07D 409/10; H10K 85/6572; H10K 85/6574; H10K 85/6576; H10K 50/15

(57) ABSTRACT

A heterocyclic compound represented by Formula 1 and an organic light-emitting device the heterocyclic compound represented by Formula 1:

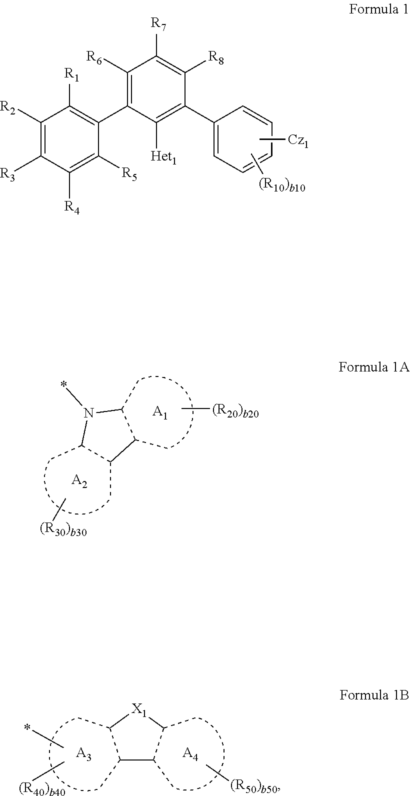

wherein, in Formula 1,
Cz₁ is a group represented by Formula 1A,
Het₁ is a group represented by Formula 1B,
wherein, R₁ to R₁₀, R₂₀, R₃₀, R₄₀, R₅₀, and b10 to b50 are each independently the same as described in the detailed description of the specification.

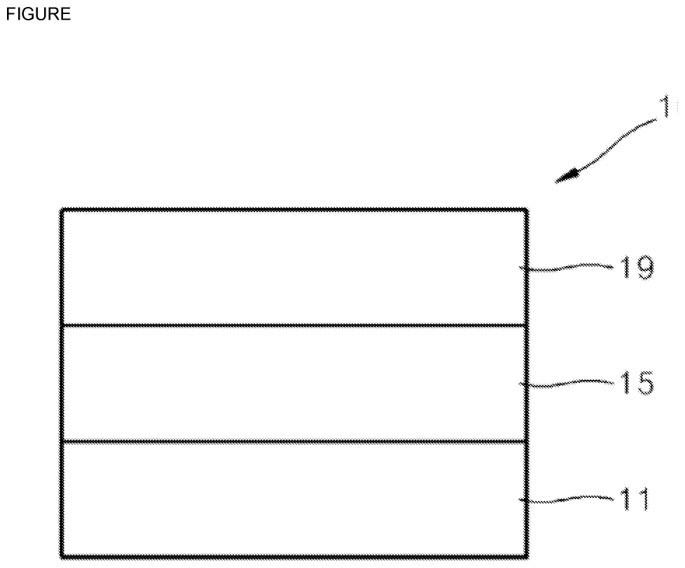

### CROSS-REFERENCE TO RELATED APPLICATION

This is a continuation application of Ser. No. 16/716,966, filed Dec. 17, 2019, which claims the priority and the benefit of Korean Patent Application No. 10-2018-0167893, filed on Dec. 21, 2018, in the Korean Intellectual Property Office, and all the benefits accruing therefrom under 35 U.S.C. 119, the contents of which are incorporated herein in their entirety by reference.

### BACKGROUND

### 1. Field

One or more embodiments relate to a heterocyclic compound and an organic light-emitting device including the same.

### 2. Description of the Related Art

Organic light-emitting devices are self-emission devices that produce full-color images, and also have wide viewing angles, high contrast ratios, short response times, and excellent characteristics in terms of brightness, driving voltage, and response speed.

In an example, an organic light-emitting device includes an anode, a cathode, and an organic layer between the anode and the cathode, wherein the organic layer includes an emission layer. A hole transport region may be between the anode and the emission layer, and an electron transport region may be between the emission layer and the cathode. Holes provided from the anode may move toward the emission layer through the hole transport region, and electrons provided from the cathode may move toward the emission layer through the electron transport region. Carriers, such as holes and electrons, recombine in the emission layer to produce excitons. These excitons transit from an excited state to a ground state, thereby generating light.

### SUMMARY

Aspects of the present disclosure provide a heterocyclic compound and an organic light-emitting device including the same.

Additional aspects will be set forth in part in the description which follows and, in part, will be apparent from the description, or may be learned by practice of the presented embodiments.

An aspect of the present disclosure provides a heterocyclic compound represented by Formula 1:

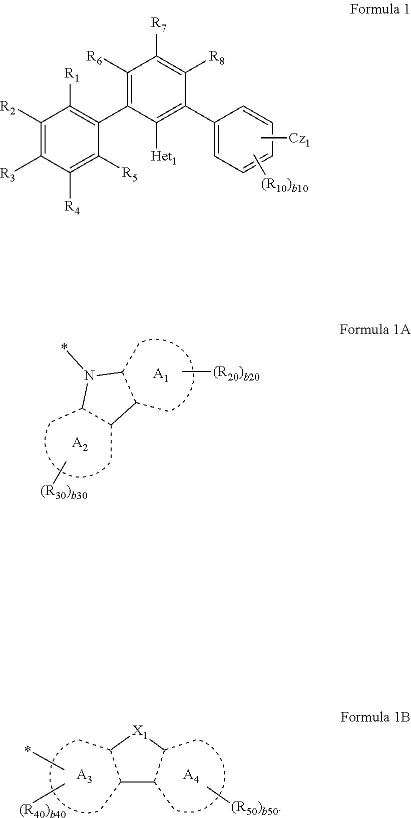

In Formula 1,
Cz₁ may be a group represented by Formula 1A,
Het₁ may be a group represented by Formula 1B,
in Formulae 1, 1A, and 1B,
X₁ may be O, S, or N(R₉),
A₁ to A₄ may each independently be a C₅-C₆₀ carbocyclic group or a C₁-C₆₀ heterocyclic group,
R₁ to R₁₀, R₂₀, R₃₀, R₄₀, and R₅₀ may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), or —B(Q₆)(Q₇),
b10 may be an integer from 1 to 4,
b20, b30, b40, and b50 may each independently be an integer from 1 to 10,
at least one substituent of the substituted C₅-C₆₀ carbocyclic group, the substituted C₁-C₆₀ heterocyclic group, the substituted C₆-C₆₀ arylene group, the substituted C₁-C₆₀ heteroarylene group, the substituted divalent non-aromatic condensed polycyclic group, the substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₄)(Q₁₅), or —B(Q₁₆)(Q₁₇);
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group;
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₄)(Q₂₅), or —B(Q₂₆)(Q₂₇); or —Si(Q₃₁)(Q₃₂)(Q₃₃), —N(Q₃₄)(Q₃₅), or —B(Q₃₆)(Q₃₇), and
Q₁ to Q₇, Q₁₁ to Q₁₇, Q₂₁ to Q₂₇, and Q₃₁ to Q₃₇ may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group.

Another aspect of the present disclosure provides an organic light-emitting device including: a first electrode; a second electrode; and an organic layer between the first electrode and the second electrode and including an emission layer, wherein the organic layer includes at least one of the heterocyclic compound represented by Formula 1.

### BRIEF DESCRIPTION OF THE DRAWING

These and/or other aspects will become apparent and more readily appreciated from the following description of the embodiments, taken in conjunction with FIGURE which is a schematic view of an organic light-emitting device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the FIGURES, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly on the other element or intervening elements may be present therebetween. In contrast, when an element is referred to as being “directly on” another element, there are no intervening elements present.

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer, or section from another element, component, region, layer, or section. Thus, “a first element,” “component,” “region,” “layer,” or “section” discussed below could be termed a second element, component, region, layer, or section without departing from the teachings herein.

The terminology used herein is for the purpose of describing particular embodiments only and is not intended to be limiting. As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise.

“Or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the FIGURES. It will be understood that relative terms are intended to encompass different orientations of the device in addition to the orientation depicted in the FIGURES.

For example, if the device in one of the FIGURES is turned over, elements described as being on the “lower” side of other elements would then be oriented on “upper” sides of the other elements. The exemplary term “lower,” can therefore, encompasses both an orientation of “lower” and “upper,” depending on the particular orientation of the FIGURE.

Similarly, if the device in one of the FIGURES is turned over, elements described as “below” or “beneath” other elements would then be oriented “above” the other elements.

The exemplary terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

A heterocyclic compound according to an embodiment is represented by Formula 1 below:

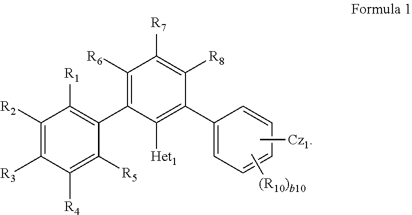

In Formula 1,
Cz₁ may be a group represented by Formula 1A, and
Het₁ may be a group represented by Formula 1B:

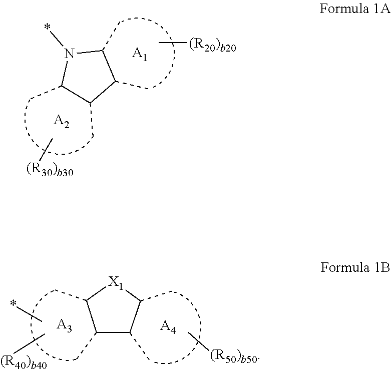

In Formula 1B, X₁ may be O, S, or N(R₉).

In Formulae 1A and 1B, A₁ to A₄ may each independently be a C₅-C₆₀ carbocyclic group or a C₁-C₆₀ heterocyclic group.

In an embodiment, A₁ to A₄ may each independently be a benzene group, a naphthalene group, a fluorene group, a pyridine group, a pyrazine group, a pyrimidine group, a pyridazine group, a carbazole group, a triazine group, a dibenzofuran group, a dibenzothiophene group, or a dibenzosilole group.

In an embodiment, A₁ to A₄ may each independently be a benzene group.

In an embodiment, in Formula 1, Cz₁ may be a group represented by Formulae 2A-1 to 2A-7:

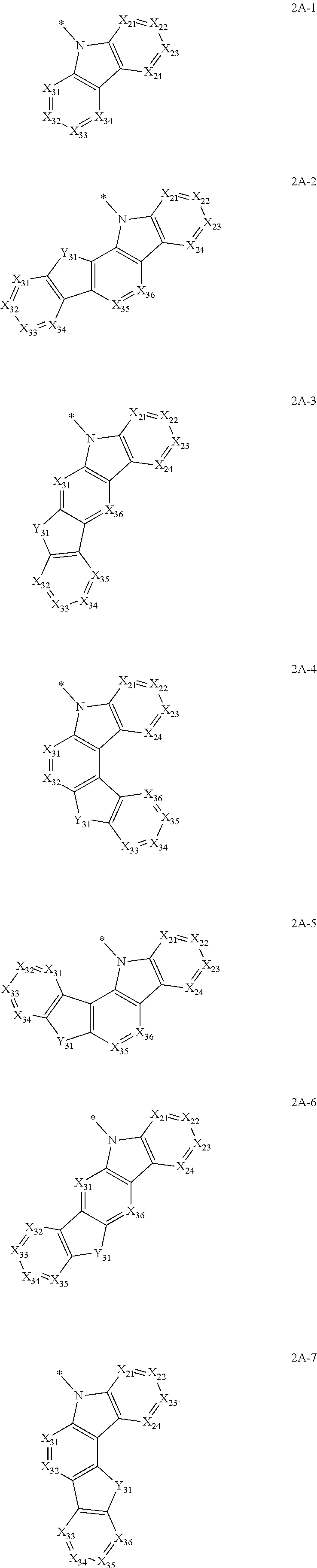

In Formulae 2A-1 to 2A-7,
X₂₁ may be N or C(R₂₁), X₂₂ may be N or C(R₂₂), X₂₃ may be N or C(R₂₃), X₂₄ may be N or C(R₂₄), and each of X₂₁ to X₂₄ may not be N,
X₃₁ may be N or C(R₃₁), X₃₂ may be N or C(R₃₂), X₃₃ may be N or C(R₃₃), X₃₄ may be N or C(R₃₄), X₃₅ may be N or C(R₃₅), X₃₆ may be N or C(R₃₆), and each of X₃₁ to X₃₆ may not be N,
Y₃₁ may be O, S, N(R₃₇), C(R₃₇)(R₃₈), or Si(R₃₇)(R₃₈), R₂₁ to R₂₄ may each independently be the same as defined in connection with R₂₀,
R₃₁ to R₃₈ may each independently be the same as defined in connection with R₃₀, and
* indicates a binding site to a neighboring atom.

In an embodiment, in Formula 1, Het₁ may be a group represented by Formulae 2B-1 to 2B-7:

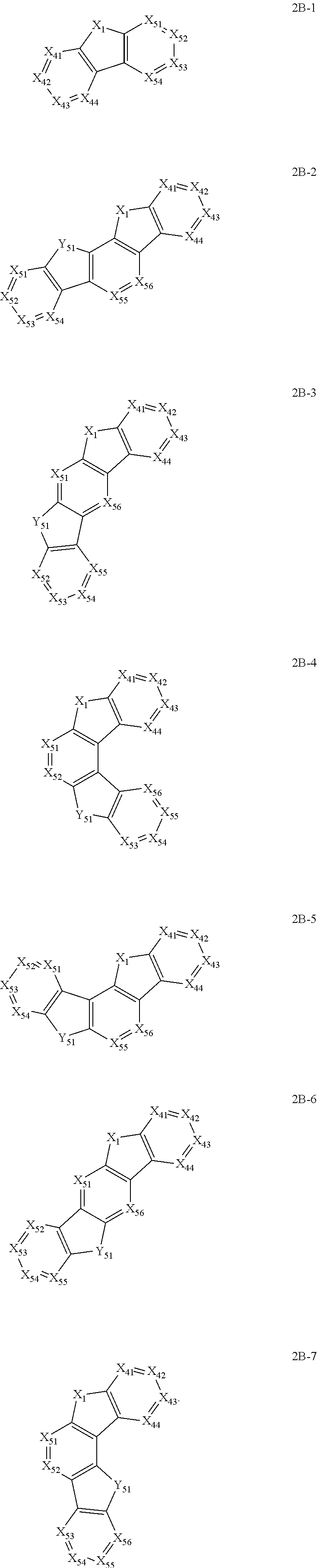

In Formulae 2B-1 to 2B-7,
X₁ is the same as described above, X₄₁ may be N, C(R₄₁), or *—C, X₄₂ may be N, C(R₄₂), or *—C, X₄₃ may be N, C(R₄₃), or *—C, X₄₄ may be N, C(R₄₄), or *—C, and one of X₄₁ to X₄₄ may be *—C,
X₆₁ may be N or C(R₅₁), X₅₂ may be N or C(R₅₂), X₅₃ may be N or C(R₅₃), X₅₄ may be N or C(R₅₄), X₅₅ may be N or C(R₅₅), X₅₆ may be N or C(R₅₆), and each of X₆₁ to X₅₆ may not be N,
Y₅₁ may be O, S, N(R₅₇), C(R₅₇)(R₅₈), or Si(R₅₇)(R₅₈),
R₄₁ to R₄₄ may each independently be the same as defined in connection with R₄₀,
R₅₁ to R₅₈ may each independently be the same as defined in connection with R₅₀, and
* indicates a binding site to a neighboring atom.

In Formulae 1, 1A, and 1B, R₁ to R₁₀, R₂₀, R₃₀, R₄₀, and R₅₀ may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), or —B(Q₆)(Q₇).

In an embodiment, R₁ to R₁₀, R₂₀, R₃₀, R₄₀, and R₅₀ may each independently be:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, or a C₁-C₂₀ alkoxy group;
a C₁-C₂₀ alkyl group or a C₁-C₂₀ alkoxy group, each substituted with at least one deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a pyridinyl group, a pyrimidinyl group, a pyrazinyl group, a pyridazinyl group, or a triazinyl group;
a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-bifluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, an imidazopyridimidinyl group, or an imidazopyridinyl group; or
a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-bifluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, an imidazopyridimidinyl group, or an imidazopyridinyl group, each substituted with at least one deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a naphthyl group, an anthracenyl group, a pyrenyl group, a phenanthrenyl group, a fluorenyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, or a quinazolinyl group.

In an embodiment, R₁ to R₁₀, R₂₀, R₃₀, R₄₀, and R₅₀ may each independently be:
hydrogen, deuterium, a cyano group, a C₁-C₂₀ alkyl group, or a C₁-C₂₀ alkoxy group;
a C₁-C₂₀ alkyl group or a C₁-C₂₀ alkoxy group, each substituted with at least one deuterium, a cyano group, a phenyl group, a biphenyl group, a terphenyl group, or a naphthyl group;
a cyclopentyl group, a cyclohexyl group, a cyclopentenyl group, a cyclohexenyl group, a cycloheptenyl group, a phenyl group, a biphenyl group, a terphenyl group, a naphthyl group, a fluorenyl group, a carbazolyl group, a dibenzofuranyl group, or a dibenzothiophenyl group; or
a cyclopentyl group, a cyclohexyl group, a cyclopentenyl group, a cyclohexenyl group, a cycloheptenyl group, a phenyl group, a biphenyl group, a terphenyl group, a naphthyl group, a fluorenyl group, a carbazolyl group, a dibenzofuranyl group, or a dibenzothiophenyl group, each substituted with at least one of deuterium, a cyano group, a C₁-C₂₀ alkyl group, a C₁-C₂₀ alkoxy group, a phenyl group, a biphenyl group, a terphenyl group, or a naphthyl group.

In an embodiment, R₁ to R₁₀, R₂₀, R₃₀, R₄₀, and R₅₀ may each independently be hydrogen, deuterium, a cyano group, a C₁-C₂₀ alkyl group, a C₁-C₂₀ alkoxy group, or a phenyl group.

In an embodiment, R₁ to R₁₀, R₂₀, R₃₀, R₄₀, and R₅₀ may each independently be hydrogen, deuterium, a cyano group, a C₁-C₂₀ alkyl group, or a C₁-C₂₀ alkoxy group.

In an embodiment, R₁ to R₁₀, R₂₀, R₃₀, R₄₀, and R₅₀ may each independently be hydrogen, a cyano group, a C₁-C₂₀ alkyl group, or a phenyl group.

In an embodiment, R₁ to R₁₀, R₂₀, R₃₀, R₄₀, and R₅₀ may each independently be hydrogen, a cyano group, a methyl group, an ethyl group, a propyl group, an isobutyl group, a sec-butyl group, ter-butyl group, or a phenyl group.

In an embodiment, the heterocyclic compound may not include a cyano group, or the heterocyclic compound may include one to three cyano groups.

In an embodiment, at least one of R₁ to R₁₀, R₂₀, R₃₀, R₄₀, and R₅₀ may be a cyano group. For example, at least one of R₁ to R₅ may be a cyano group. For example, R₂ may be a cyano group. In an embodiment, R₄ may be a cyano group. In an embodiment, R₂₀ may be a cyano group. In an embodiment, R₅₀ may be a cyano group.

In Formulae 1, 1A, and 1B, b10 may be an integer from 1 to 4, and b20, b30, b40, and b50 may each independently be an integer from 1 to 10.

In an embodiment, b10 may be an integer from 1 to 4, and b20, b30, b40, and b50 may each independently be an integer from 1 to 4.

In an embodiment, the heterocyclic compound represented by Formula 1 may be represented by Formula 10:

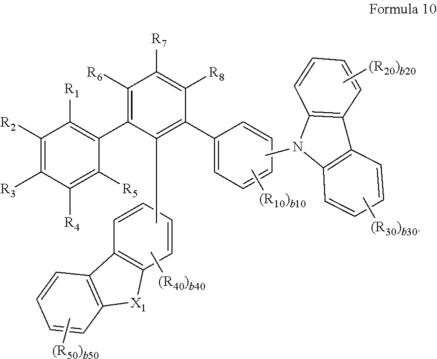

In Formula 10,
X₁, R₁ to R₁₀, R₂₀, R₃₀, R₄₀, and R₅₀ are each independently the same as described above,
b20, b30, and b50 may each independently be an integer from 1 to 4, and
b40 may be an integer from 1 to 3.

In an embodiment, the heterocyclic compound represented by Formula 1 may be represented by one of Formulae 10-1 to 10-12:

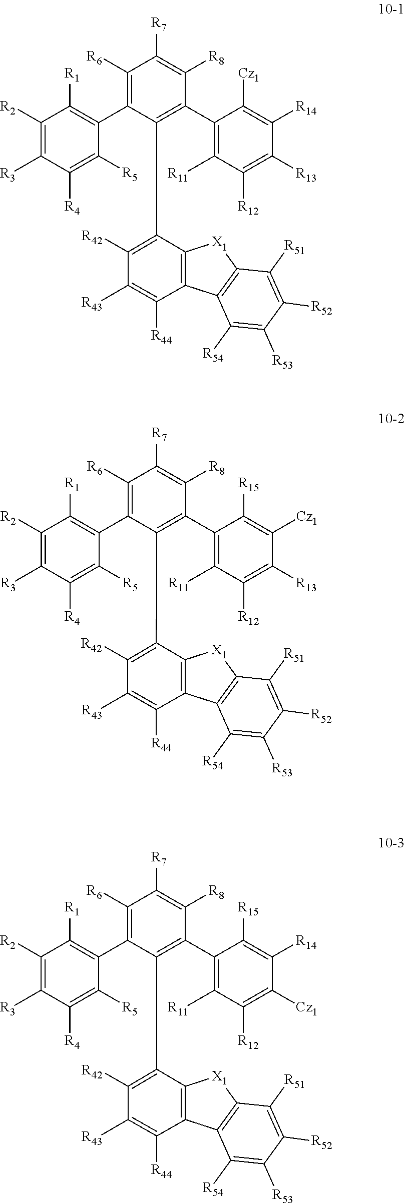

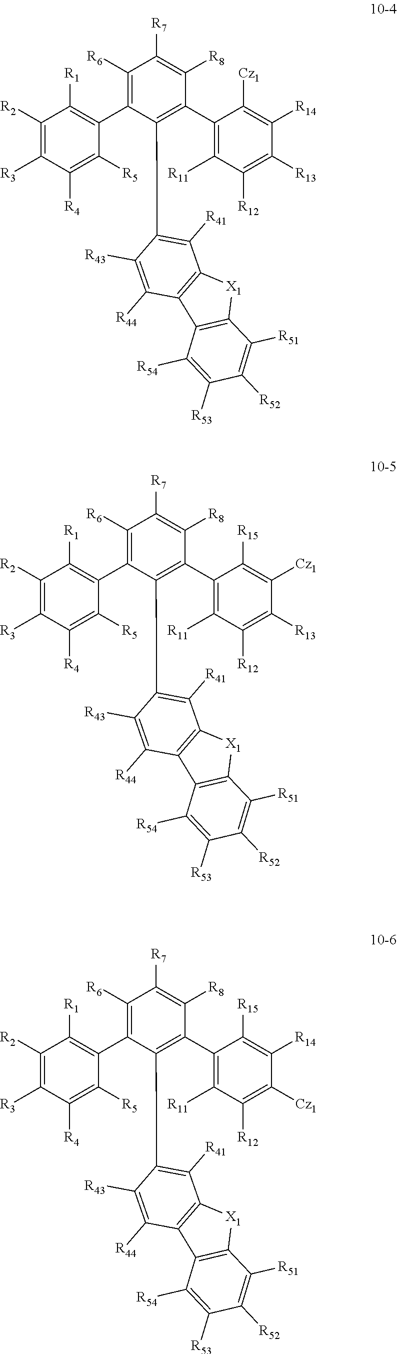

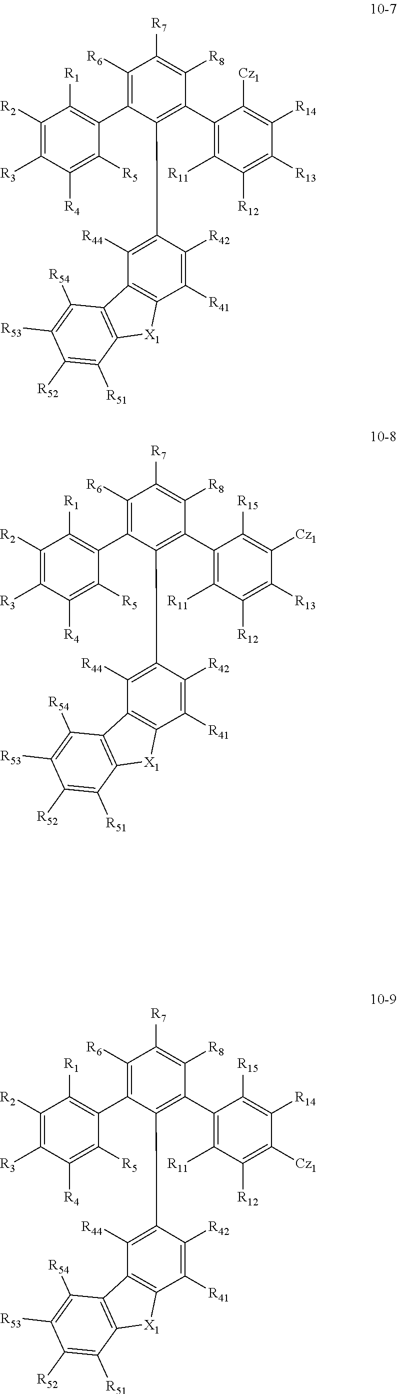

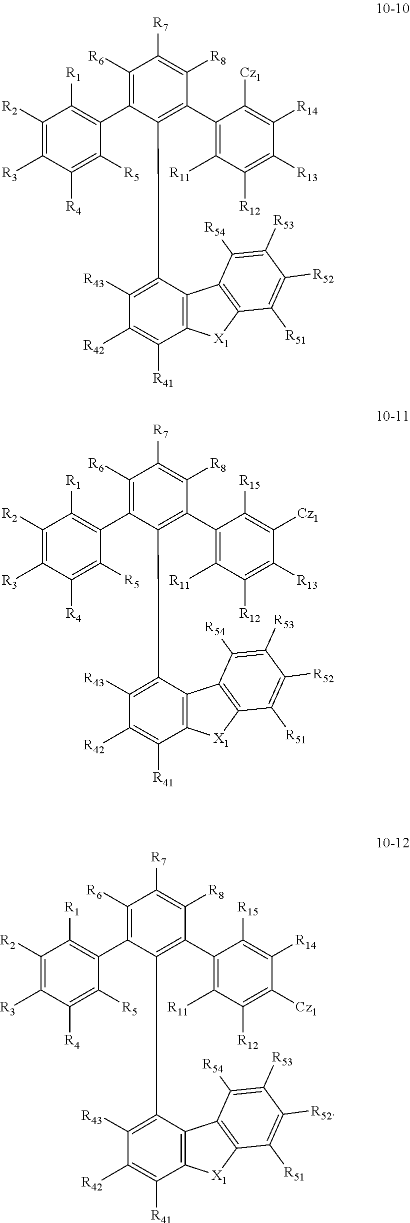

In Formulae 10-1 to 10-12,
Cz₁ may be a group represented by Formula 10A:

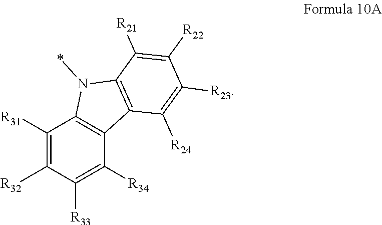

In Formulae 10-1 to 10-12 and 10A,
X₁ and R₁ to R₉ are each independently the same as described above,
R₁₁ to R₁₅ may each independently be the same as defined in connection with R₁₀,
R₂₁ to R₂₄ may each independently be the same as defined in connection with R₂₀,
R₃₁ to R₃₄ may each independently be the same as defined in connection with R₃₀,
R₄₁ to R₄₄ may each independently be the same as defined in connection with R₄₀,
R₅₁ to R₅₄ may each independently be the same as defined in connection with R₅₀, and
* indicates a binding site to a neighboring atom.

In an embodiment, at least one of R₁ to R₅ may be a cyano group. For example, R₂ may be a cyano group. In an embodiment, R₄ may be a cyano group.

In an embodiment, at least one of R₂₁ to R₂₄ may be a cyano group. For example, R₂₃ may be a cyano group.

In an embodiment, at least one of R₅₁ to R₅₄ may be a cyano group. For example, R₅₃ may be a cyano group.

At least one substituent of the substituted C₅-C₆₀ carbocyclic group, the substituted C₁-C₆₀ heterocyclic group, the substituted C₆-C₆₀ arylene group, the substituted C₁-C₆₀ heteroarylene group, the substituted divalent non-aromatic condensed polycyclic group, the substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₄)(Q₁₅), or —B(Q₁₆)(Q₁₇);
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group;
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₄)(Q₂₅), or —B(Q₂₆)(Q₂₇), or
—Si(Q₃₁)(Q₃₂)(Q₃₃), —N(Q₃₄)(Q₃₅), or —B(Q₃₆)(Q₃₇), and
Q₁ to Q₇, Q₁₁ to Q₁₇, Q₂₁ to Q₂₇, and Q₃₁ to Q₃₇ may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group.

In an embodiment, the heterocyclic compound may have an asymmetrical structure.

In an embodiment, the heterocyclic compound may be one of Compounds 1 to 15:

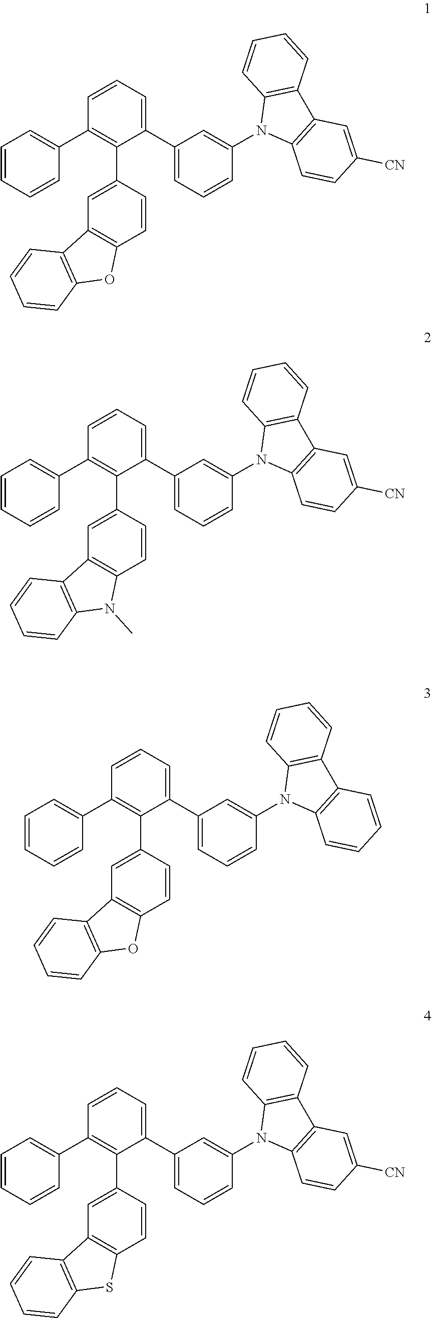 [SMILES: N#Cc1ccc2c(c1)c1ccccc1n2-c1cccc(-c2cccc(-c3ccccc3)c2-c2ccc3oc4ccccc4c3c2)c1 ; Cn1c2ccccc2c2cc(-c3c(-c4ccccc4)cccc3-c3cccc(-n4c5ccccc5c5cc(C#N)ccc54)c3)ccc21 ; c1ccc(-c2cccc(-c3cccc(-n4c5ccccc5c5ccccc54)c3)c2-c2ccc3oc4ccccc4c3c2)cc1 ; N#Cc1ccc2c(c1)c1ccccc1n2-c1cccc(-c2cccc(-c3ccccc3)c2-c2ccc3sc4ccccc4c3c2)c1]

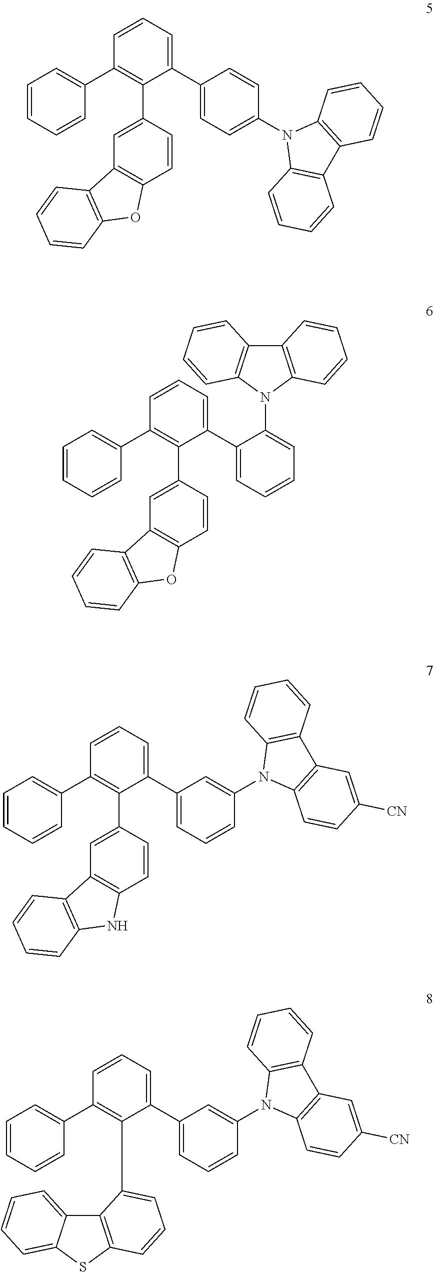 [SMILES: c1ccc(-c2cccc(-c3ccc(-n4c5ccccc5c5ccccc54)cc3)c2-c2ccc3oc4ccccc4c3c2)cc1 ; c1ccc(-c2cccc(-c3ccccc3-n3c4ccccc4c4ccccc43)c2-c2ccc3oc4ccccc4c3c2)cc1 ; N#Cc1ccc2c(c1)c1ccccc1n2-c1cccc(-c2cccc(-c3ccccc3)c2-c2ccc3[nH]c4ccccc4c3c2)c1 ; N#Cc1ccc2c(c1)c1ccccc1n2-c1cccc(-c2cccc(-c3ccccc3)c2-c2cccc3sc4ccccc4c23)c1]

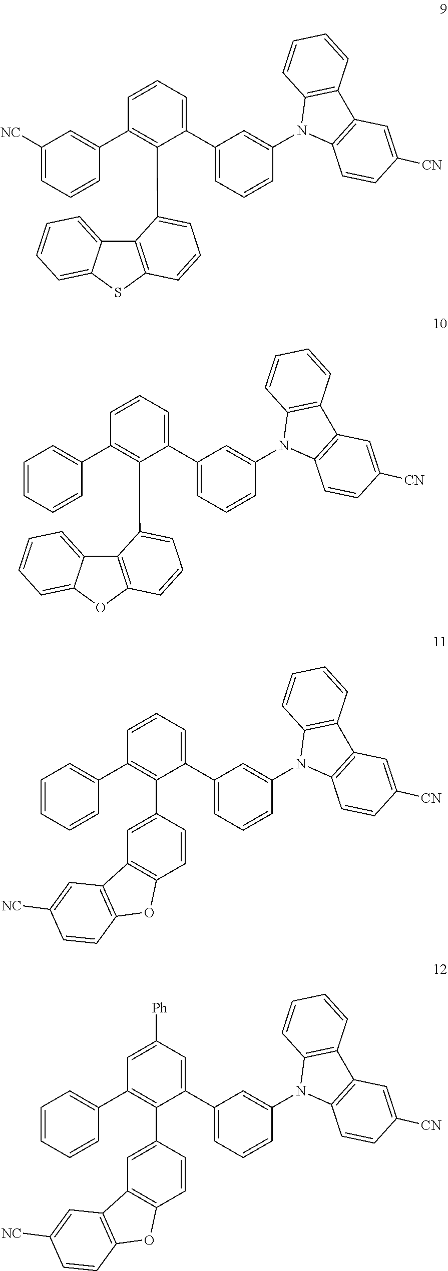 [SMILES: N#Cc1cccc(-c2cccc(-c3cccc(-n4c5ccccc5c5cc(C#N)ccc54)c3)c2-c2cccc3sc4ccccc4c23)c1 ; N#Cc1ccc2c(c1)c1ccccc1n2-c1cccc(-c2cccc(-c3ccccc3)c2-c2cccc3oc4ccccc4c23)c1 ; N#Cc1ccc2oc3ccc(-c4c(-c5ccccc5)cccc4-c4cccc(-n5c6ccccc6c6cc(C#N)ccc65)c4)cc3c2c1 ; N#Cc1ccc2oc3ccc(-c4c(-c5ccccc5)cc(-c5ccccc5)cc4-c4cccc(-n5c6ccccc6c6cc(C#N)ccc65)c4)cc3c2c1]

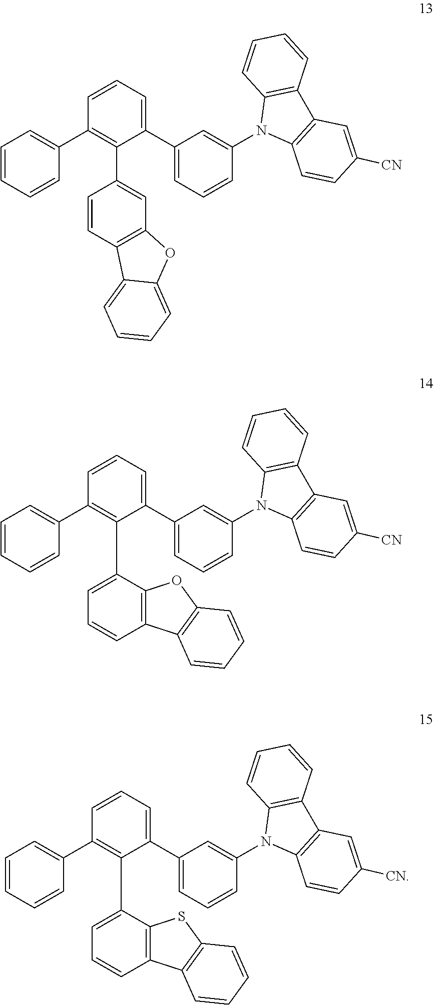 [SMILES: N#Cc1ccc2c(c1)c1ccccc1n2-c1cccc(-c2cccc(-c3ccccc3)c2-c2ccc3c(c2)oc2ccccc23)c1 ; N#Cc1ccc2c(c1)c1ccccc1n2-c1cccc(-c2cccc(-c3ccccc3)c2-c2cccc3c2oc2ccccc23)c1 ; N#Cc1ccc2c(c1)c1ccccc1n2-c1cccc(-c2cccc(-c3ccccc3)c2-c2cccc3c2sc2ccccc23)c1]

In Compounds 1 to 15, -Ph indicates a phenyl group.

The heterocyclic compound represented by Formula 1 may include an m-terphenyl group linker. A substituent represented by Het₁ in Formula 1 is substituted at a specific position of a medium benzene ring of the m-terphenyl group linker. Hence, steric hindrance occurs and a conjugation length is shortened. Therefore, since the compound represented by Formula 1 may have a relatively high T₁ energy level, the compound represented by Formula 1 may have relatively excellent electron and hole transport capabilities.

In addition, in the heterocyclic compound represented by Formula 1, a carbazole group is N-substituted in an outer benzene ring of the m-terphenyl linker. Therefore, the heterocyclic compound represented by Formula 1 may have a high T₁ energy level and excellent hole transport capability.

In addition, since the heterocyclic compound represented by Formula 1 may include a cyano group, the heterocyclic compound represented by Formula 1 may have excellent electron transport capability and may adjust the highest occupied molecular orbital (HOMO) and lowest unoccupied molecular orbital (LUMO).

As described above, the heterocyclic compound represented by Formula 1 may have electric characteristics suitable for a material for manufacturing an organic light-emitting device, for example, a host material, a hole transport material, or an electron transport material in an emission layer. Therefore, an organic light-emitting device including the heterocyclic compound may have high efficiency and/or a long lifespan.

For example, the HOMO, LUMO, and T₁ energy levels of Compounds 1-15 and Comparative Compound 1 were evaluated by using a DFT method of Gaussian program (structurally optimized at a level of B3LYP, 6-31G(d,p)), and the results thereof are shown in Table 1.

TABLE 1

| | HOMO | LUMO | T₁ |
|---|---|---|---|
| Compound No. | (eV) | (eV) | (eV) |
| Compound 1 | −5.680 | −1.213 | 3.13 |
| Compound 2 | −5.40 | −1.05 | 3.12 |
| Compound 3 | −5.31 | −1.07 | 3.16 |
| Compound 4 | −5.70 | −1.22 | 3.09 |
| Compound 5 | −5.26 | −1.07 | 3.14 |
| Compound 6 | −5.26 | −0.90 | 3.15 |
| Compound 7 | −5.46 | −1.09 | 3.12 |
| Compound 8 | −5.73 | −1.23 | 3.09 |
| Compound 9 | −5.79 | −1.56 | 3.09 |
| Compound 10 | −5.73 | −1.22 | 3.12 |
| Compound 11 | −5.86 | −1.63 | 3.12 |
| Compound 12 | −5.83 | −1.59 | 3.06 |
| Compound 13 | −5.72 | −1.27 | 3.04 |
| Compound 14 | −5.71 | −1.23 | 3.12 |
| Compound 15 | −5.71 | −1.24 | 3.09 |
| Comparative Compound 1 | −5.419 | −1.066 | 3.00 |
| 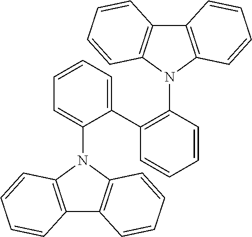 [SMILES: c1ccc(-n2c3ccccc3c3ccccc32)c(-c2ccccc2-n2c3ccccc3c3ccccc32)c1] | | | |

Referring to Table 1, it is confirmed that Compound 1 as one of the heterocyclic compounds represented by Formula 1 has a high T, energy level, and the heterocyclic compound represented by Formula 1 is suitable for use as a material for manufacturing an electronic device, for example, an emission layer of an organic light-emitting device.

Synthesis methods of the heterocyclic compound represented by Formula 1 may be understood by one of ordinary skill in the art by referring to Synthesis Examples provided below.

The heterocyclic compound represented by Formula 1 may be suitable for use as an organic layer of an organic light-emitting device, and for example, may be used as a material for forming an emission layer, a material for forming a hole transport region, and/or a material for forming an electron transport region, wherein such an emission layer, a hole transport region, and an electron transport region are included in an organic layer. Therefore, according to another aspect of the present disclosure, an organic light-emitting device includes: a first electrode, a second electrode, and an organic layer located between the first electrode and the second electrode and including an emission layer and at least one heterocyclic compound represented by Formula 1.

The organic light-emitting device may have, due to the inclusion of an organic layer including the heterocyclic compound represented by Formula 1, low driving voltage, high efficiency, high luminance, high quantum emission efficiency, and a long lifespan.

In an embodiment, in the organic light-emitting device,
the first electrode may be an anode,
the second electrode may be a cathode,
the organic layer may include a hole transport region located between the first electrode and the emission layer and an electron transport region located between the emission layer and the second electrode,
the hole transport region may include a hole injection layer, a hole transport layer, an electron blocking layer, or any combination thereof, and
the electron transport region may include a hole blocking layer, an electron transport layer, an electron injection layer, or any combination thereof, but embodiments of the present disclosure are not limited thereto.

For example, the emission layer of the organic light-emitting device may include at least one of the heterocyclic compounds represented by Formula 1.

In an embodiment, the emission layer may include a host and a dopant, wherein the host may include the heterocyclic compound represented by Formula 1, and an amount of the host may be larger than that of the dopant.

In an embodiment, the emission layer may emit blue light having a maximum emission wavelength in a range of about 410 nm to about 490 nm.

In an embodiment, the emission layer of the organic light-emitting device may include a host and a dopant, wherein the host may include the heterocyclic compound represented by Formula 1, and the dopant may include a phosphorescent dopant or a fluorescent dopant. For example, the dopant may include a phosphorescent dopant (for example, an organometallic compound represented by Formula 81). The host may further include a host compound other than the heterocyclic compound represented by Formula 1.

The emission layer may emit red light, green light, or blue light.

In an embodiment, the emission layer may be a phosphorescent dopant, but embodiments of the present disclosure are not limited thereto.

In an embodiment, the heterocyclic compound represented by Formula 1 may be included in the hole transport region of the organic light-emitting device.

For example, the hole transport region of the organic light-emitting device may include at least one of a hole injection layer, a hole transport layer, or an electron blocking layer, and at least one of the hole injection layer, the hole transport layer, or the electron blocking layer may include the heterocyclic compound represented by Formula 1.

In an embodiment, the heterocyclic compound represented by Formula 1 may be included in the electron transport region of the organic light-emitting device.

For example, the electron transport region of the organic light-emitting device may include at least one of a hole blocking layer, an electron transport layer, or an electron injection layer, and at least one of the hole blocking layer, the electron transport layer, or the electron injection layer may include the heterocyclic compound represented by Formula 1.

In an embodiment, the hole transport region of the organic light-emitting device may include the electron blocking layer, wherein the electron blocking layer includes the heterocyclic compound represented by Formula 1. The electron blocking layer may directly contact the emission layer.

In an embodiment, the electron transport region of the organic light-emitting device may include the hole blocking layer, wherein the hole blocking layer includes the heterocyclic compound represented by Formula 1. The hole blocking layer may be in direct contact with the emission layer.

In an embodiment, the organic layer of the organic light-emitting device may further include, in addition to the heterocyclic compound represented by Formula 1, an organometallic compound represented by Formula 81:

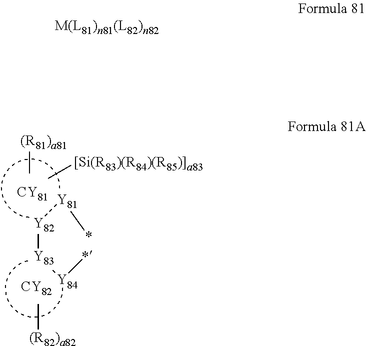

In Formulae 81 and 81A,
M may be iridium (Ir), platinum (Pt), osmium (Os), titanium (Ti), zirconium (Zr), hafnium (Hf), europium (Eu), terbium (Tb), thulium (Tm), or rhodium (Rh),
L₈₁ may be a ligand represented by Formula 81A, and n91 may be an integer from m1 to 3, wherein, when n81 is two or more, two or more L₈₁(s) may be identical to or different from each other,
L₈₂ may be an organic ligand, and n82 may be an integer from 0 to 4, wherein, when n82 is two or more, two or more L₈₂(s) may be identical to or different from each other,
Y₈₁ to Y₈₄ may each independently be carbon (C) or nitrogen (N),
Y₈₁ and Y₈₂ may be linked via a single bond or a double bond, and Y₈₃ and Y₈₄ may be linked via a single bond or a double bond, and
CY₈₁ and CY₈₂ may each independently be a C₅-C₃₀ carbocyclic group or a C₂-C₃₀ heterocarbocyclic group.

In an embodiment, CY₈₁ and CY₈₂ may optionally be linked via an organic linking group,
R₈₁ to R₈₅ may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₈₁)(Q₈₂)(Q₈₃), —N(Q₈₄)(Q₈₅), —B(Q₈₆)(Q₈₇), or —P(═O)(Q₈₈)(Q₈₉),
a81 to a83 may each independently be an integer from 0 to 5, wherein,
when a81 is two or more, two or more R₈₁(s) may be identical to or different from each other,
when a82 is two or more, two or more R₈₂(s) may be identical to or different from each other, when a81 is two or more, two neighboring R₈₁(s) may optionally be linked to form a saturated or unsaturated C₂-C₃₀ ring (for example, a benzene ring, a cyclopentane ring, a cyclohexane ring, a cyclopentene ring, a cyclohexene ring, a norbornane ring, a naphthalene ring, a benzoindene ring, a benzoindole ring, a benzofuran ring, a benzothiophene ring, a pyridine ring, a pyrimidine ring, or a pyrazine ring), or a saturated or unsaturated C₂-C₃₀ ring substituted with at least one R₈₈ (for example, a benzene ring, a cyclopentane ring, a cyclohexane ring, a cyclopentene ring, a cyclohexene ring, a norbornane ring a naphthalene ring, a benzoindene ring, a benzoindole ring, a benzofuran ring, a benzothiophene ring, a pyridine ring, a pyrimidine ring, or a pyrazine ring, each substituted with at least one R₈₈),
when a82 is two or more, two neighboring R₈₂(s) may optionally be linked to form a saturated or unsaturated C₂-C₃₀ ring (for example, a benzene ring, a cyclopentane ring, a cyclohexane ring, a cyclopentene ring, a cyclohexene ring, a norbornane ring, a naphthalene ring, a benzoindene ring, a benzoindole ring, a benzofuran ring, a benzothiophene ring, a pyridine ring, a pyrimidine ring, or a pyrazine ring) or a saturated or unsaturated C₂-C₃₀ ring substituted at least one R₈₉ (for example, a benzene ring, a cyclopentane ring, a cyclohexane ring, a cyclopentene ring, a cyclohexene ring, a norbornane ring, a bicyclo[2.2.1]heptane ring, a naphthalene ring, a benzoindene ring, a benzoindole ring, a benzofuran ring, a benzothiophene ring, a pyridine ring, a pyrimidine ring, or a pyrazine ring, each substituted with at least one R₈₉),
R₈₈ may be the same as defined in connection with R₈₁,
R₈₉ may be the same as defined in connection with R₈₂,
* and *′ in Formula 81A each indicate a binding site to M in Formula 81, at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, or —Si(Q₉₁)(Q₉₂)(Q₉₃), and
Q₈₁ to Q₈₉ and Q₉₁ to Q₉₃ may each independently be hydrogen, deuterium, a C₁-C₆₀ alkyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₁-C₆₀ heteroaryl group, a monovalent non-aromatic condensed polycyclic group, or a monovalent non-aromatic condensed heteropolycyclic group.

In an embodiment, in Formula 81A,
a83 may be 1 or 2, and
R₈₃ to R₈₅ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂;
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group; or
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group, each substituted with at least one deuterium, a C₁-C₁₀ alkyl group, or a phenyl group,
but embodiments of the present disclosure are not limited thereto.

In an embodiment, in Formula 81A,
Y₈₁ may be nitrogen,
Y₈₂ and Y₈₃ may each independently be carbon,
Y₈₄ may be nitrogen or carbon, and
CY₈₁ and CY₈₂ may each independently be a cyclopentadiene group, a benzene group, a heptalene group, an indene group, a naphthalene group, an azulene group, a heptalene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentacene group, a rubicene group, a corozene group, an ovalene group, a pyrrole group, an isoindole group, an indole group, an indazole group, a pyrazole group, an imidazole group, a triazole group, an oxazole group, an isoxazole group, an oxadiazole group, a thiazole group, an isothiazole group, a thiadiazole group, a purine group, a furan group, a thiophene group, a pyridine group, a pyrimidine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, a benzofuran group, a benzothiophene group, an isobenzothiazole group, a benzoxazole group, an isobenzoxazole group, a benzocarbazole group, a dibenzocarbazole group, an imidazopyridine group, an imidazopyrimidine group, a dibenzofuran group, a dibenzothiophene group, a dibenzothiophene sulfone group, a carbazole group, a dibenzosilole group, or a 2,3-dihydro-1H-imidazole group.

In an embodiment, in Formula 81A,
Y₈₁ may be nitrogen,
Y₈₂ to Y₈₄ may each independently be carbon,
CY₈₁ may be a 5-membered ring including two nitrogen atoms as a ring-forming atom, and
CY₈₂ may be a benzene group, a naphthalene group, a fluorene group, a dibenzofuran group, or a dibenzothiophene group, but embodiments of the present disclosure are not limited thereto.

In an embodiment, in Formula 81A,
Y₈₁ may be nitrogen,
Y₈₂ to Y₈₄ may each independently be carbon,
CY₈₁ may be an imidazole group or a 2,3-dihydro-1H-imidazole group, and
CY₈₂ may be a benzene group, a naphthalene group, a fluorene group, a dibenzofuran group, or a dibenzothiophene group, but embodiments of the present disclosure are not limited thereto.

In an embodiment, in Formula 81A,
Y₈₁ may be nitrogen,
Y₈₂ to Y₈₄ may each independently be carbon,
CY₈₁ may be a pyrrole group, a pyrazole group, an imidazole group, a triazole group, an oxazole group, an isoxazole group, an oxadiazole group, a thiazole group, an isothiazole group, a thiadiazole group, a pyridine group, a pyrimidine group, a quinoline group, an isoquinoline group, a benzoquinoline group, a phthalazine group, a naphthyridine group, a quinoxaline group, a quinazoline group, a cinnoline group, a benzimidazole group, an isobenzothiazole group, a benzoxazole group, or an isobenzoxazole group, and
CY₈₂ may be cyclopentadiene group, a benzene group, a naphthalene group, a fluorene group, a benzofluorene group, a dibenzofluorene group, a phenanthrene group, an anthracene group, a triphenylene group, a pyrene group, a chrysene group, a perylene group, a benzofuran group, a benzothiophene group, a benzocarbazole group, a dibenzocarbazole group, a dibenzofuran group, a dibenzothiophene group, a dibenzothiophene sulfone group, a carbazole group or a dibenzosilole group.

In an embodiment, in Formula 81A,
R₈₁ and R₈₂ may each independently be:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, or a C₁-C₂₀ alkoxy group;
a C₁-C₂₀ alkyl group and a C₁-C₂₀ alkoxy group, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a pyridinyl group, or a pyrimidinyl group;
a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, or an imidazopyrimidinyl group;
a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, or an imidazopyrimidinyl group, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, or an imidazopyrimidinyl group; or
—B(Q₈₆)(Q₈₇) or —P(═O)(Q₈₈)(Q₈₉), and
Q₈₆ to Q₈₉ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂;
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group; or
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group, each substituted with at least one deuterium, a C₁-C₁₀ alkyl group, or a phenyl group.

In an embodiment, in Formula 81A, at least one R₈₁ in the number of a81 and R₈₂ in the number of a82 may be a cyano group.

In an embodiment, in Formula 81A, at least one R₈₂ in the number of a82 may be a cyano group.

In an embodiment, in Formula 81A, at least one R₈₁ in the number of a81 and R₈₂ in the number of a82 may be deuterium.

In an embodiment, in Formula 81, L₈₂ may be a ligand represented by one of Formulae 3-1(1) to 3-1(60), 3-1(61) to 3-1(69), 3-1(71) to 3-1(79), 3-1(81) to 3-1(88), 3-1(91) to 3-1(98), and 3-1(101) to 3-1(114):

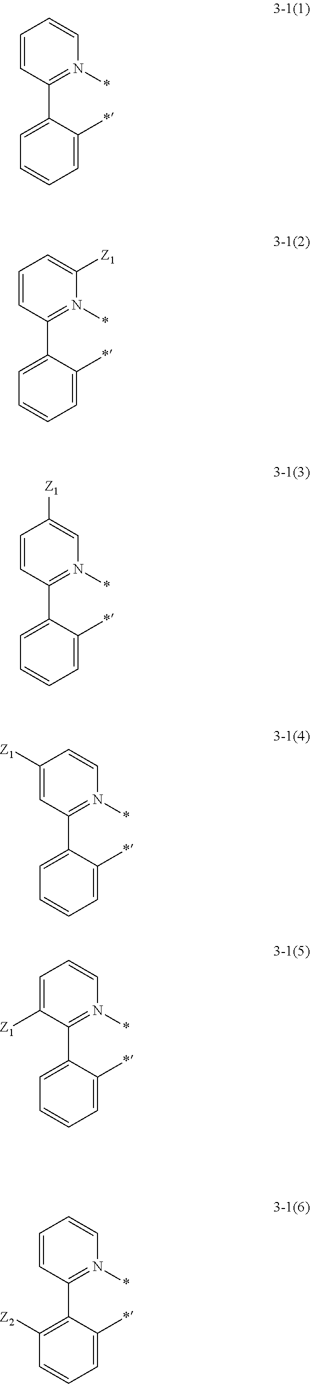

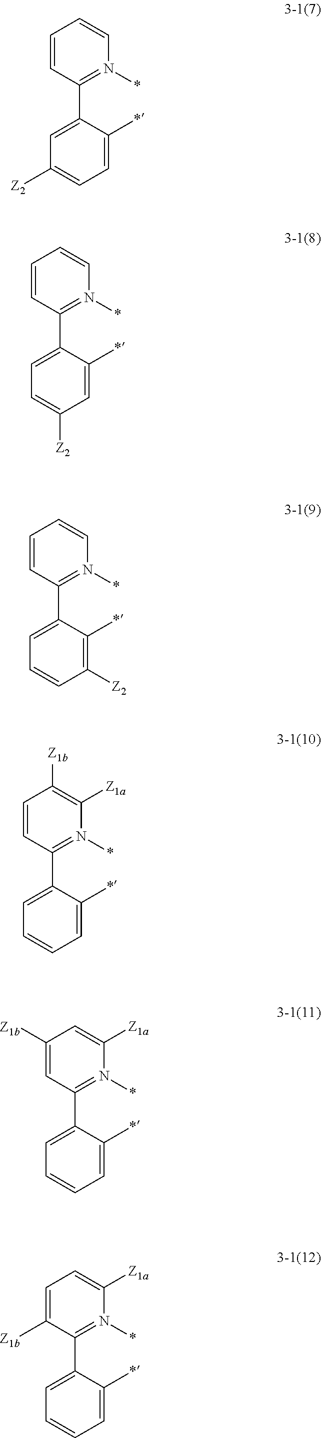

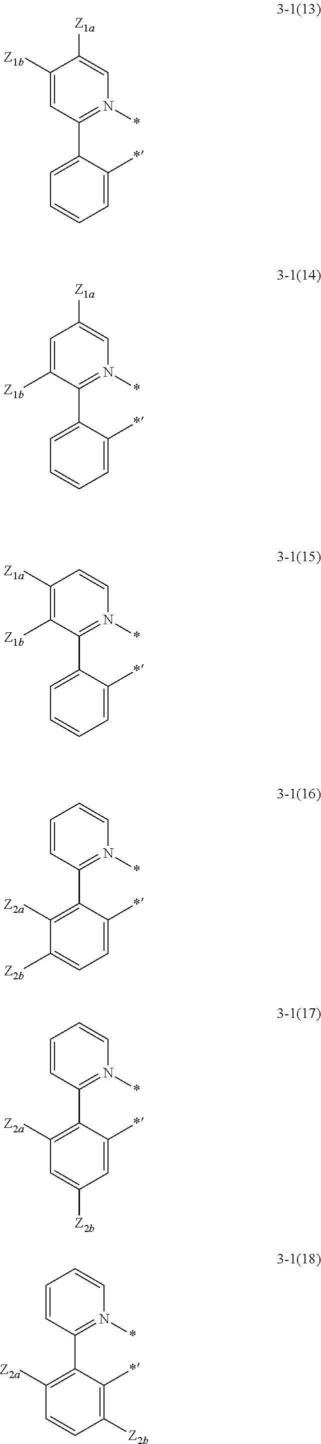

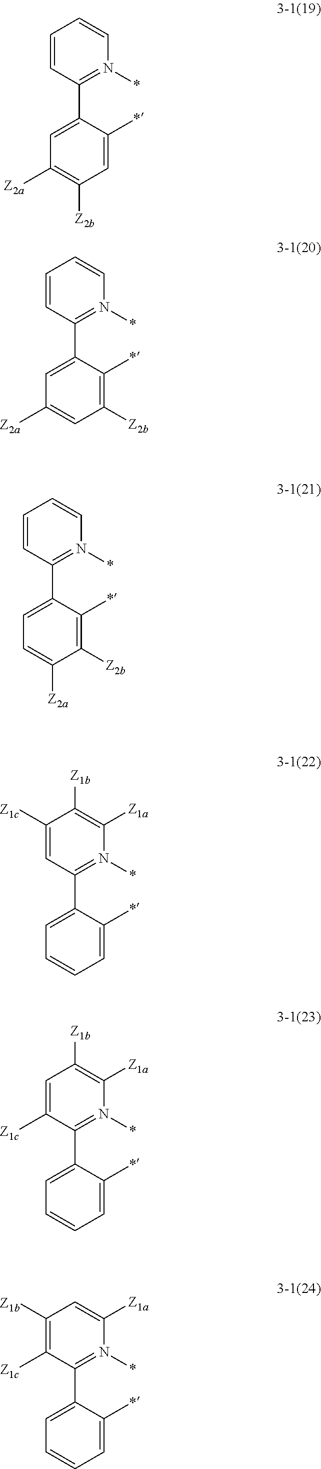

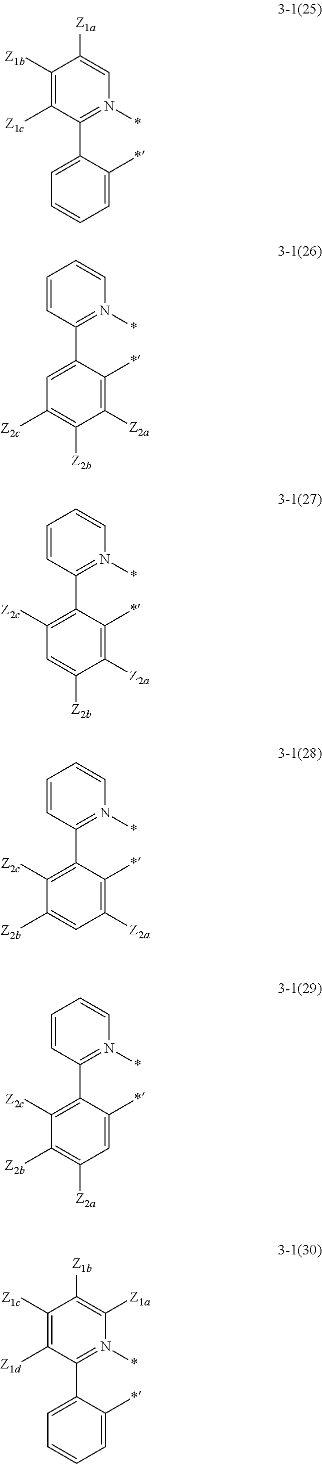

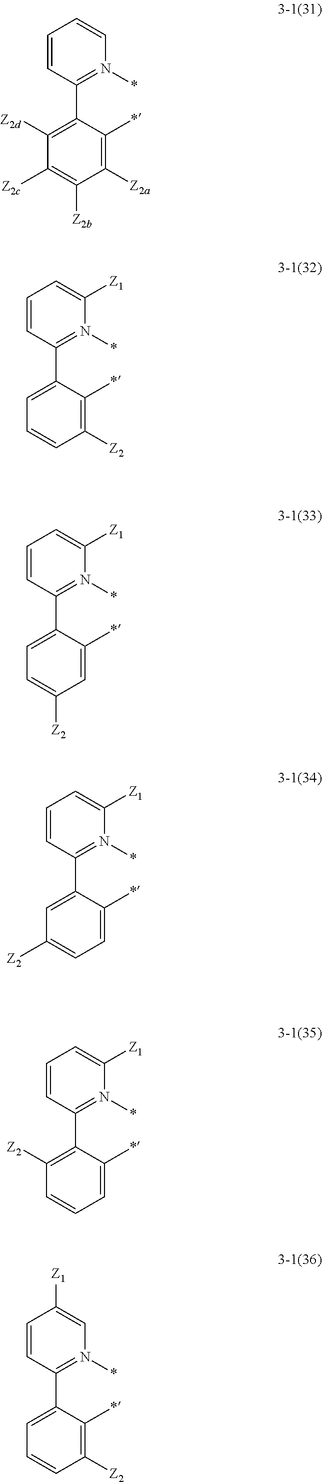

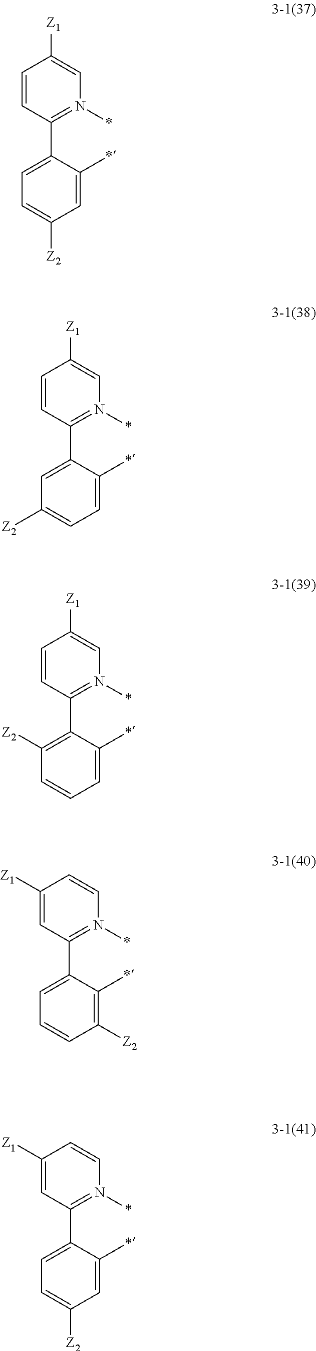

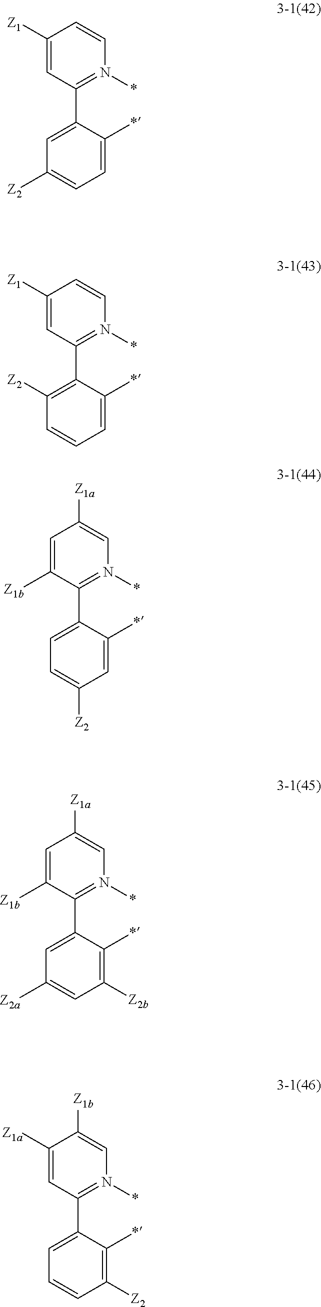

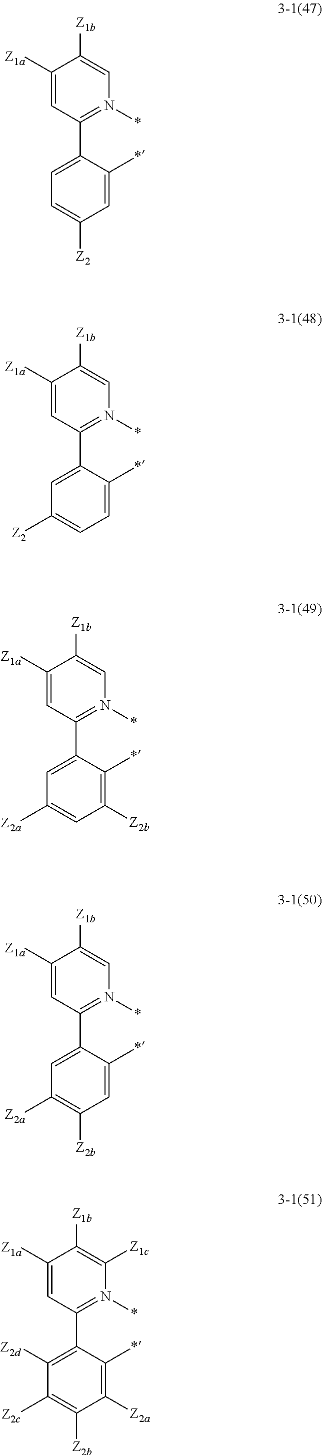

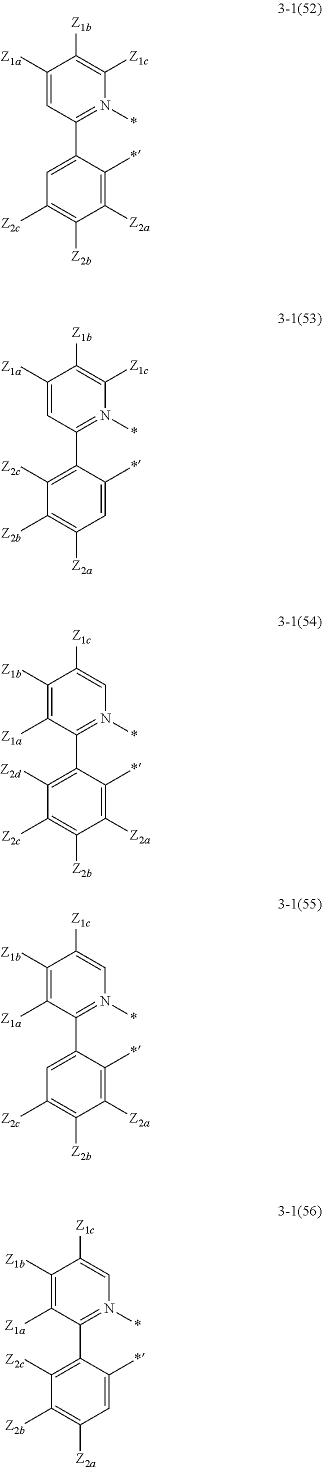

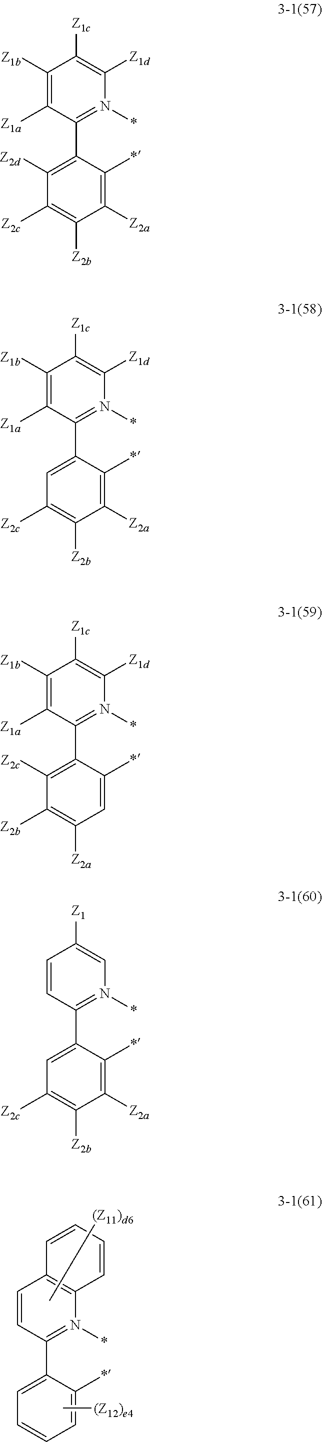

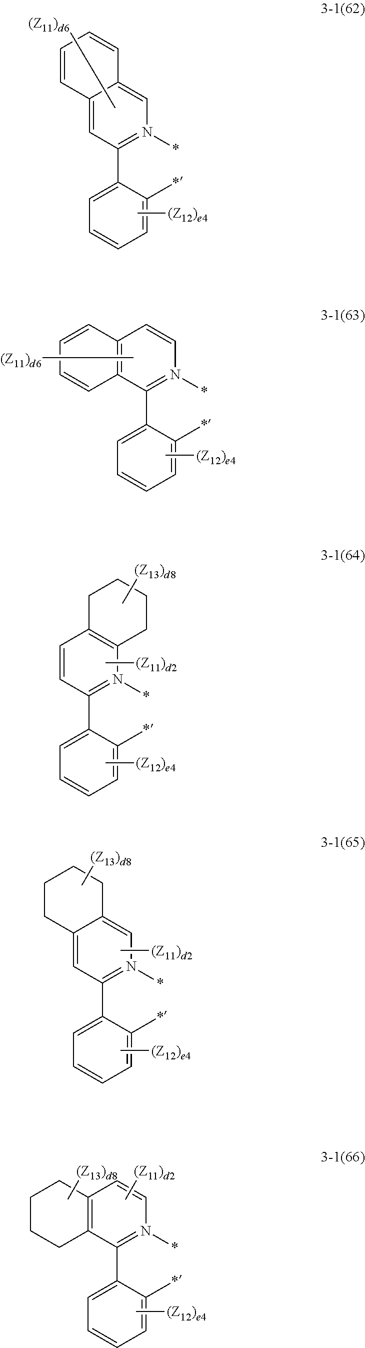

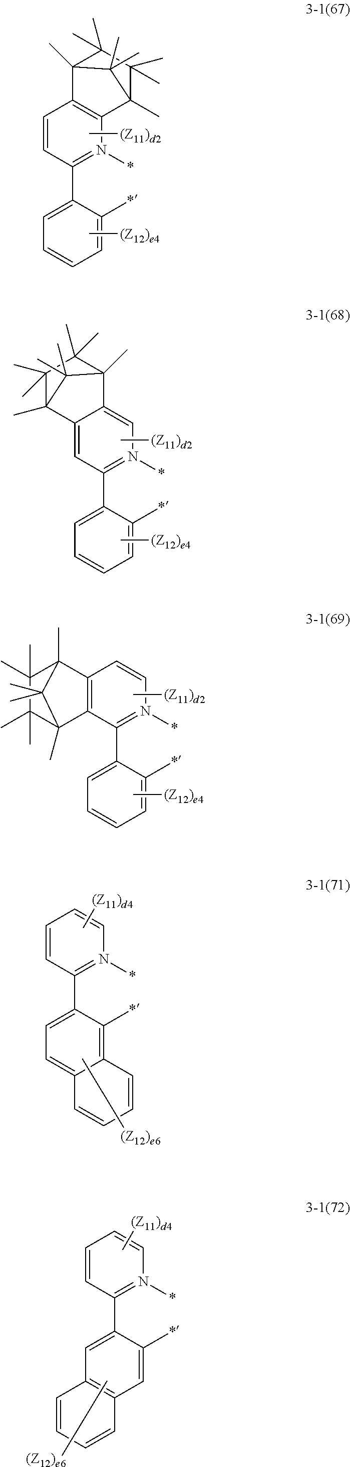

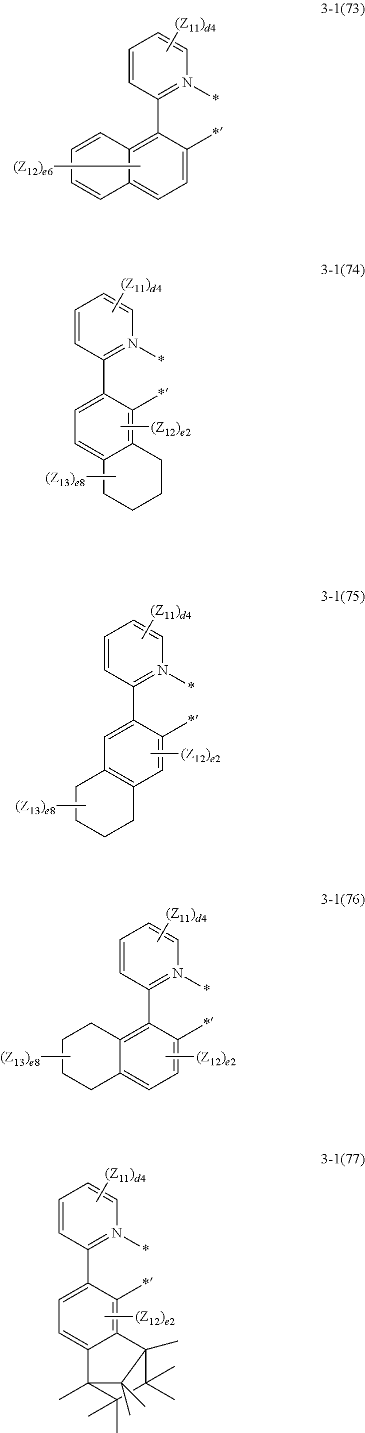

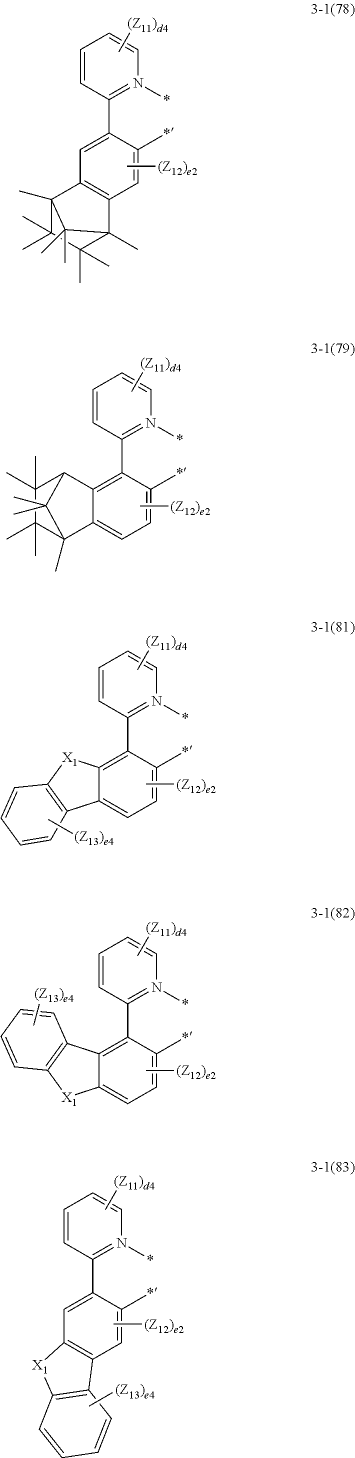

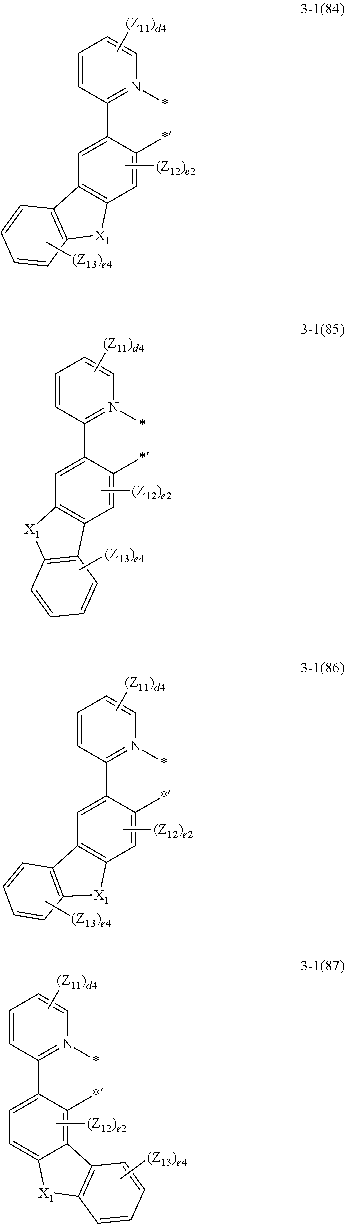

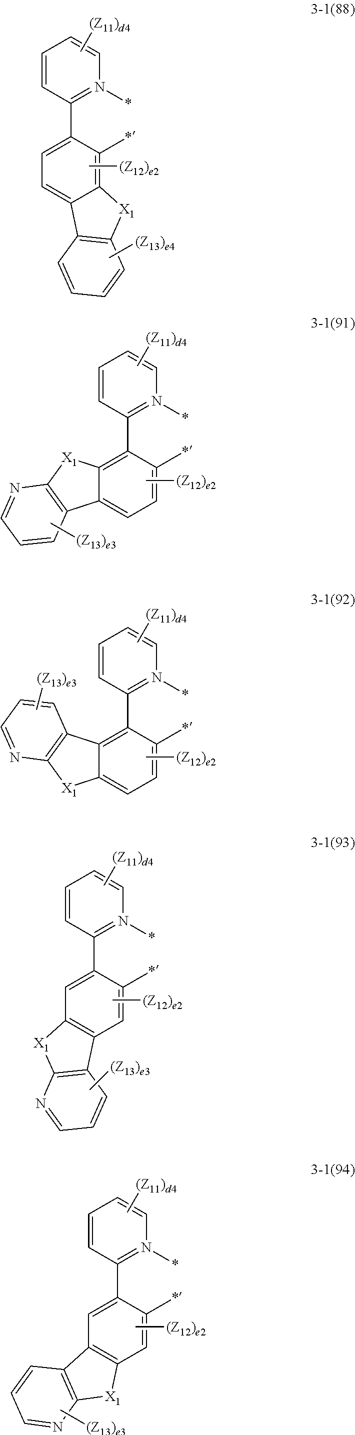

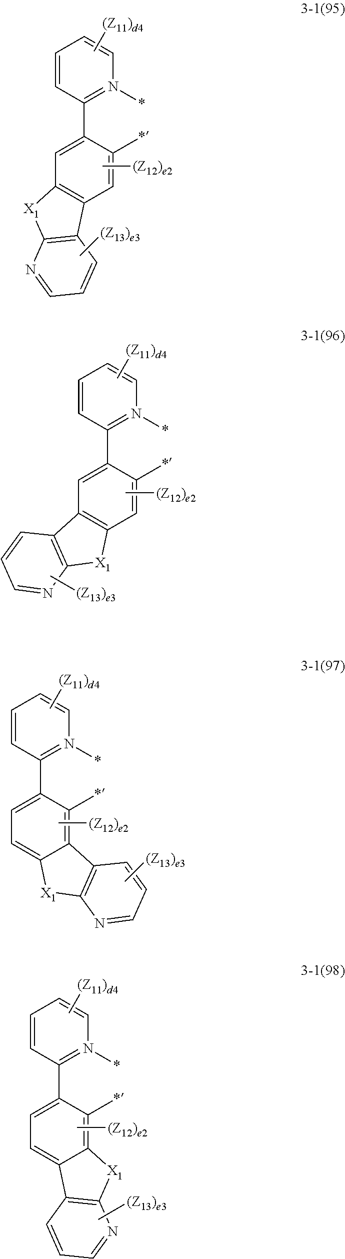

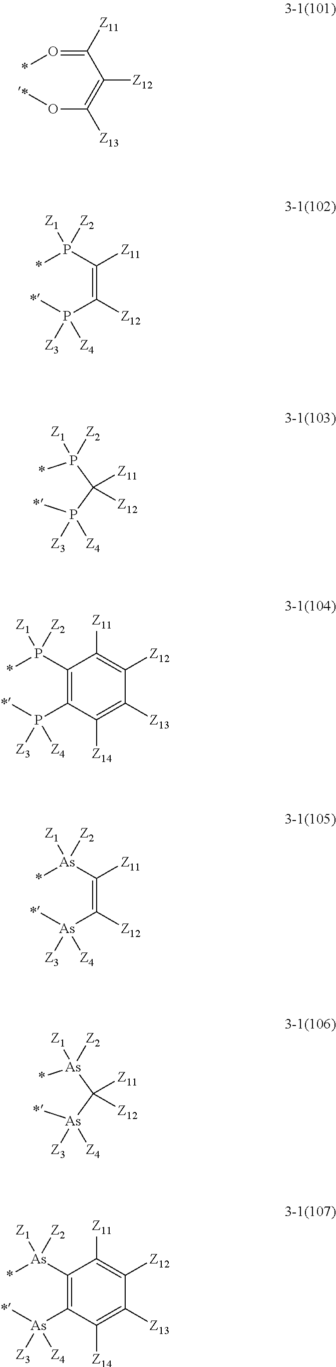

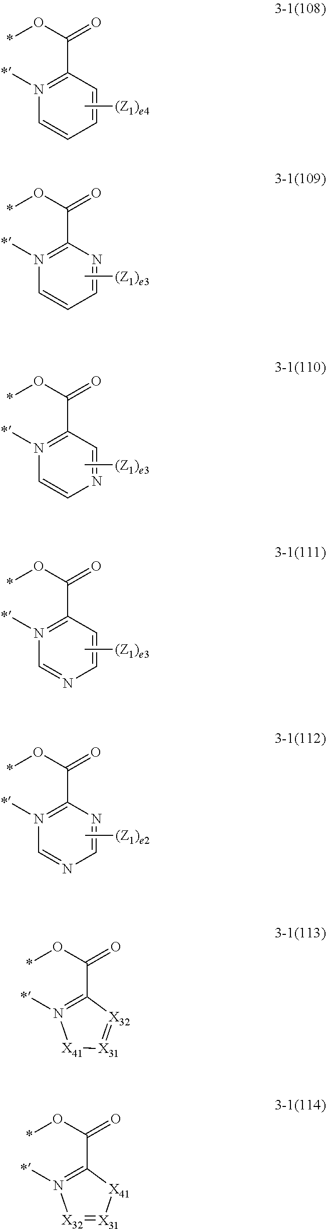

In Formulae 3-1(1) to 3-1(60), 3-1(61) to 3-1(69), 3-1(71) to 3-1(79), 3-1(81) to 3-1(88), 3-1(91) to 3-1(98), and 3-1(101) to 3-1(114),
X₁ may be O, S, C(Z₂₁)(Z₂₂), or N(Z₂₃),
X₃₁ may be N or C(Z₁ₐ),
X₃₂ may be N or C(Z_{1b}),
X₄₁ may be O, S, N(Z₁ₐ), or C(Z₁ₐ)(Z_{1b}),
Z₁ to Z₄, Z₁ₐ, Z_{1b}, Z_{1c}, Z_{1d}, Z₂ₐ, Z_{2b}, Z_{2c}, Z_{2d}, Z₁₁ to Z₁₄, and Z₂₁ to Z₂₃ may each independently be:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, or a C₁-C₂₀ alkoxy group;
a C₁-C₂₀ alkyl group or a C₁-C₂₀ alkoxy group, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a pyridinyl group, or a pyrimidinyl group;
a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, or an imidazopyrimidinyl group;
a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, or an imidazopyrimidinyl group, each substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, or an imidazopyrimidinyl group; or
—B(Q₈₆)(Q₈₇) or —P(═O)(Q₈₈)(Q₈₉),
Q₈₆ to Q₈₉ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂;
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group; or
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group, each substituted with at least one deuterium, a C₁-C₁₀ alkyl group, or a phenyl group,
d2 and e2 may each independently be 0 or 2,
e3 may be an integer from 0 to 3,
d4 and e4 may each independently be an integer from 0 to 4,
d6 and e6 may each independently be an integer from 0 to 6,
d8 and e8 may each independently be an integer from 0 to 8, and
* and *′ each indicate a binding site to M in Formula 1.

In an embodiment, in Formula 81, M may be Ir, and n81+n82 may be 3; or M may be Pt, and n81+n82 may be 2.

In an embodiment, the organometallic compound represented by Formula 81 may not be a salt including a pair of a cation and an anion, but instead may be a neutral compound.

In an embodiment, the organometallic compound represented by Formula 81 may include at least one of Compounds PD1 to PD78 and FIr₆, but embodiments of the present disclosure are not limited thereto:

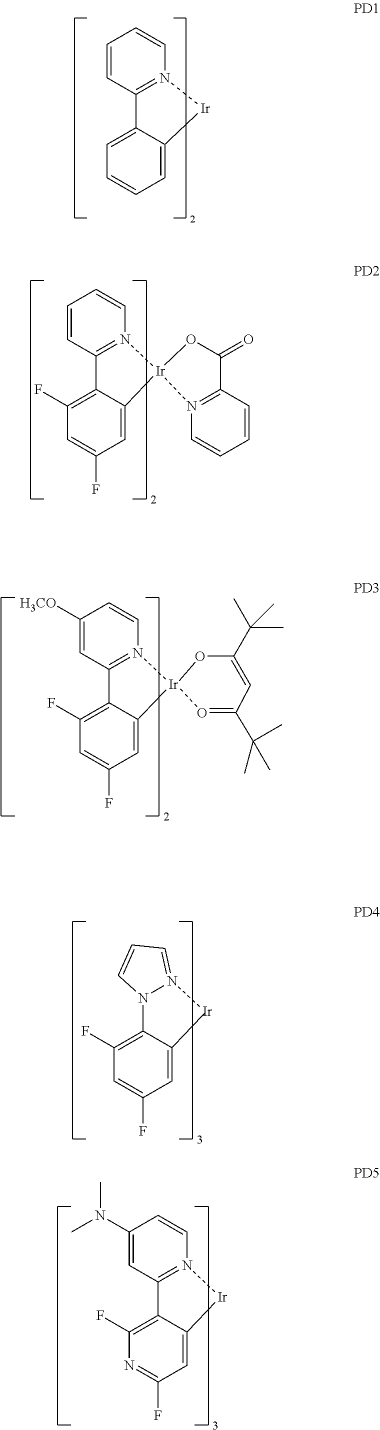

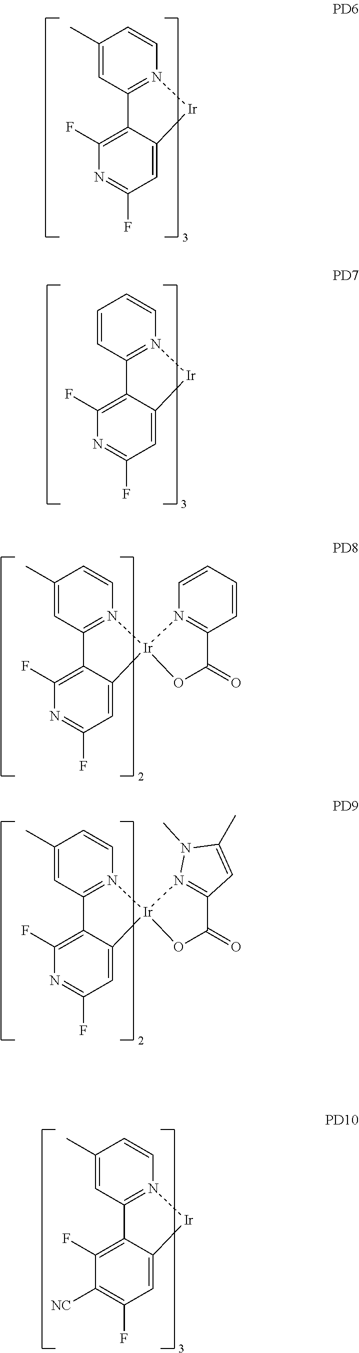

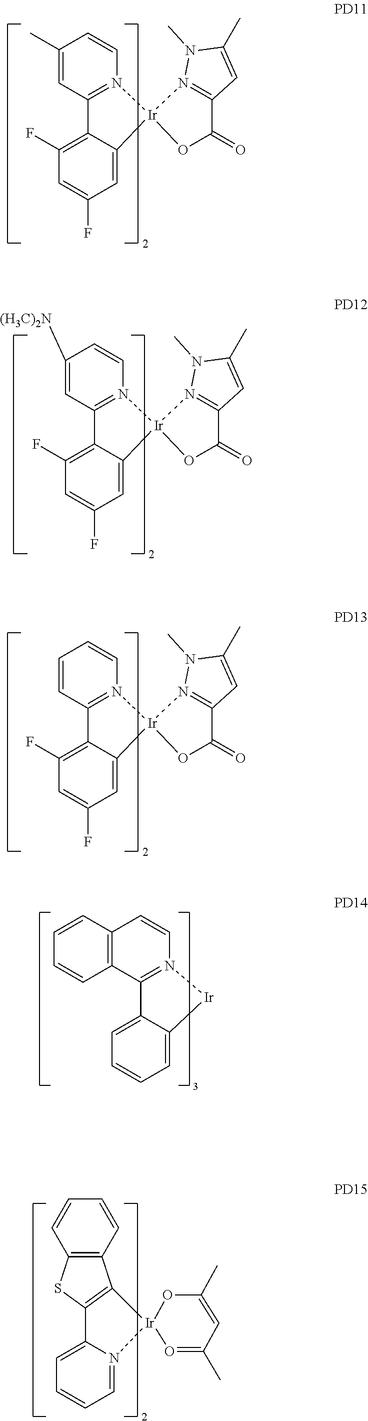

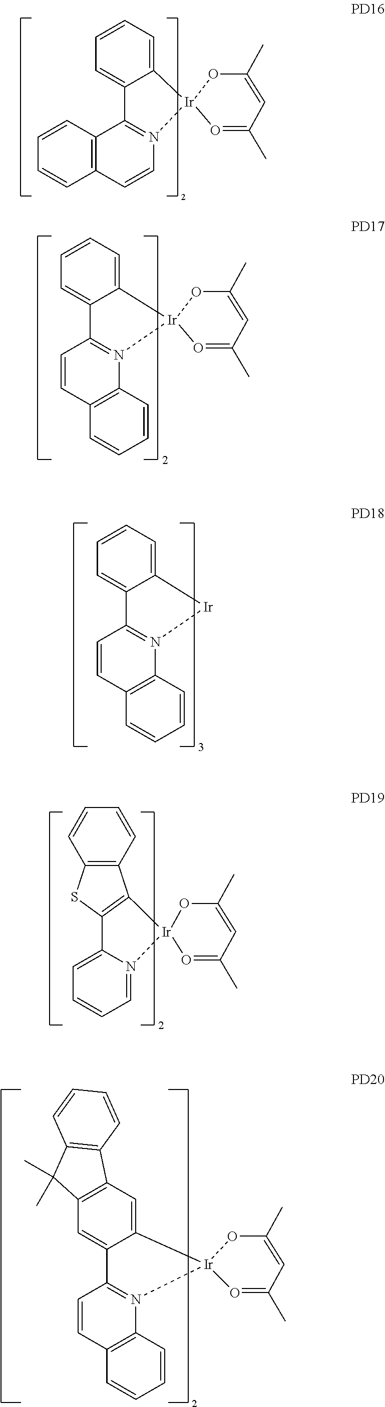

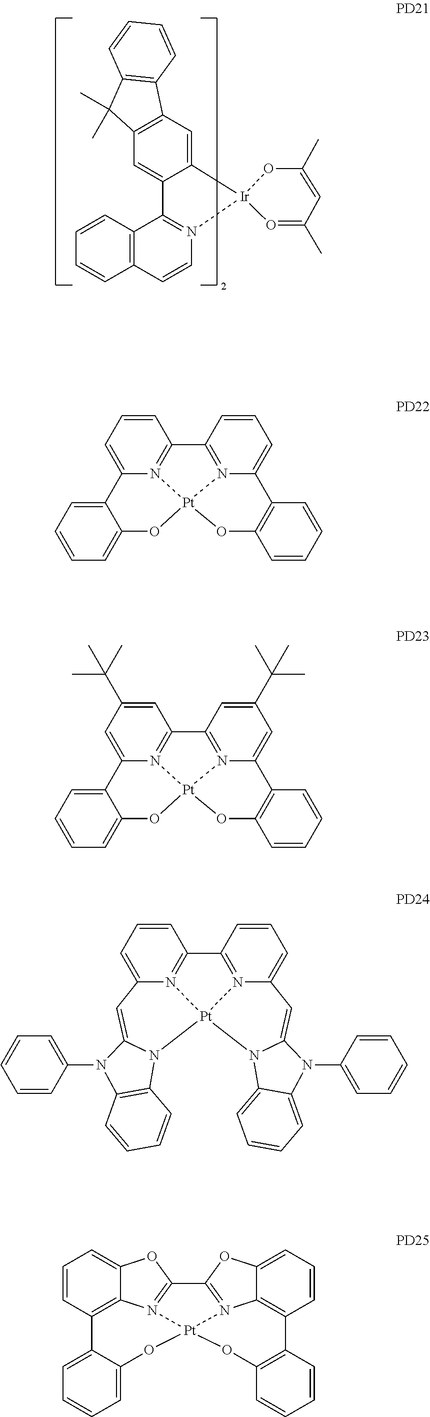

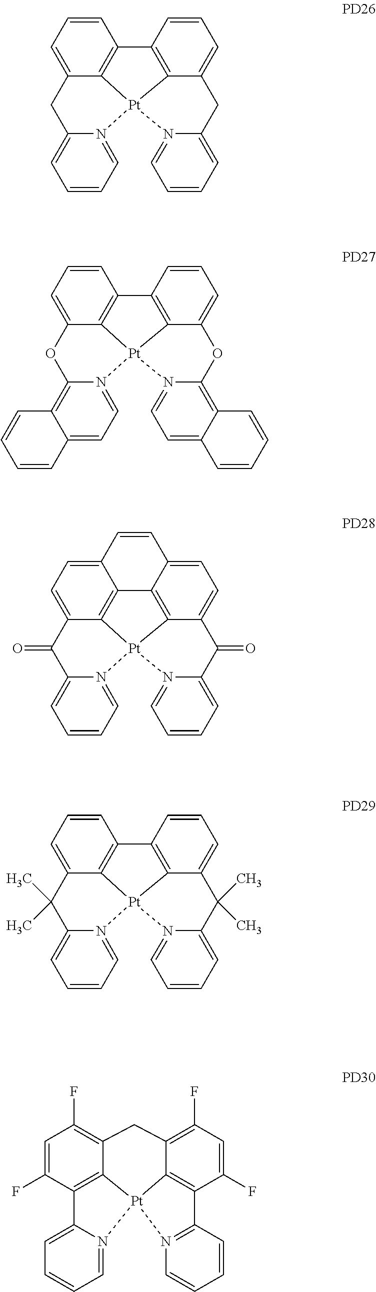

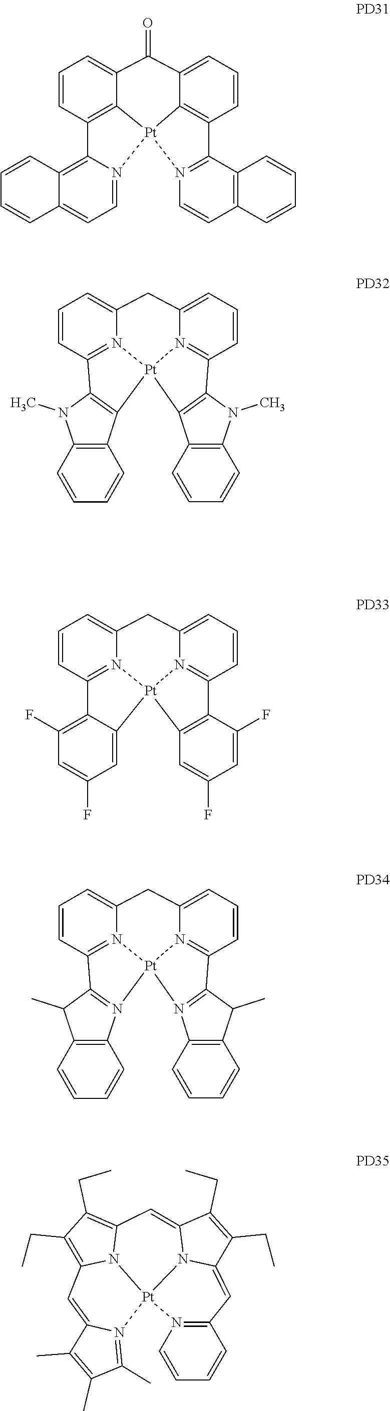

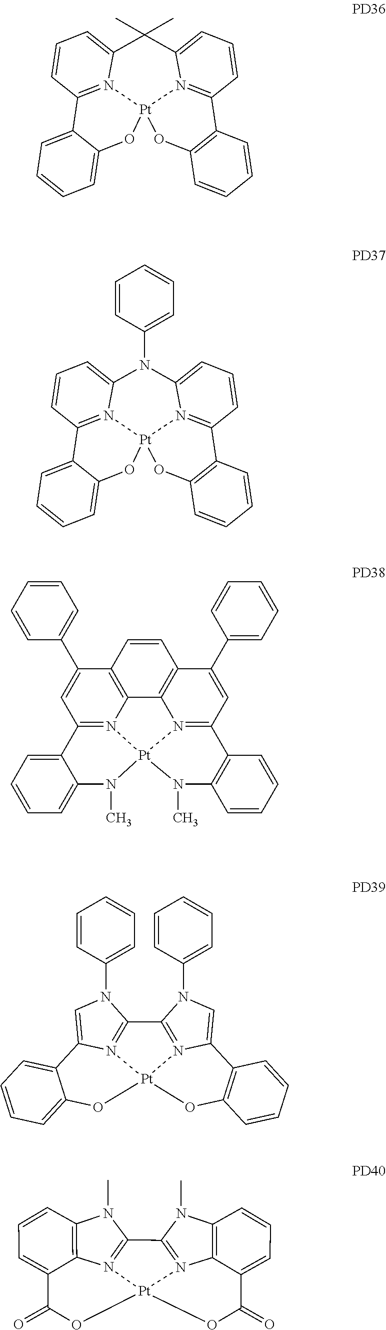

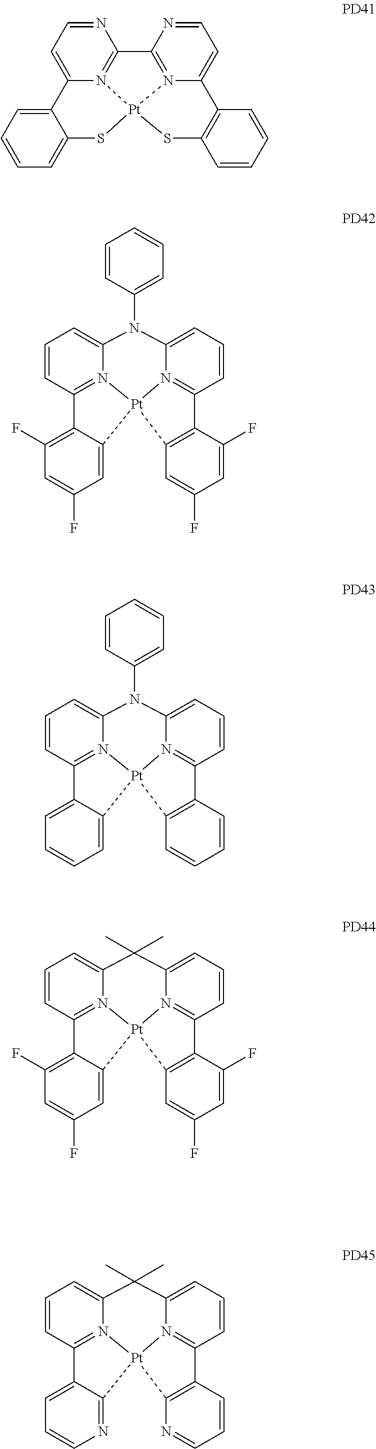

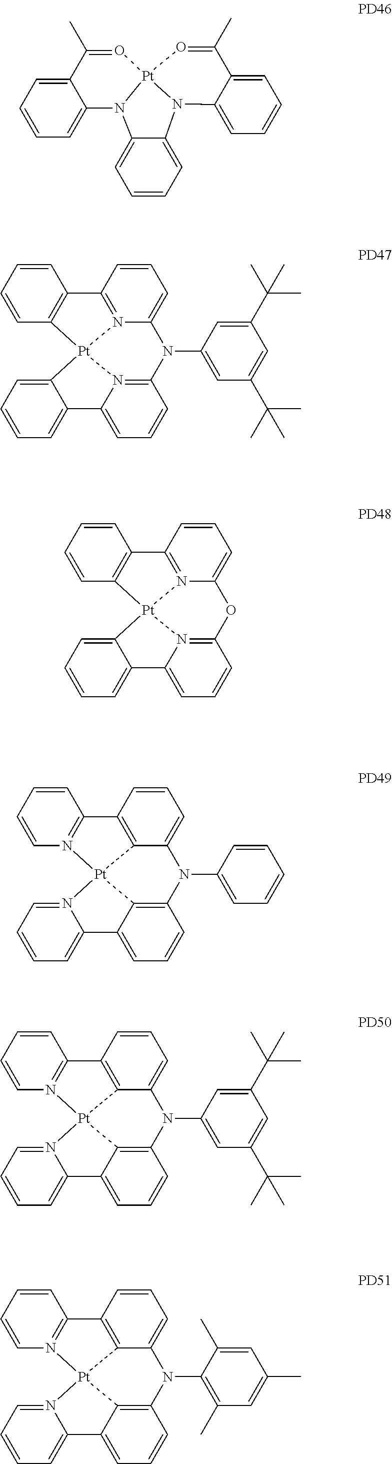

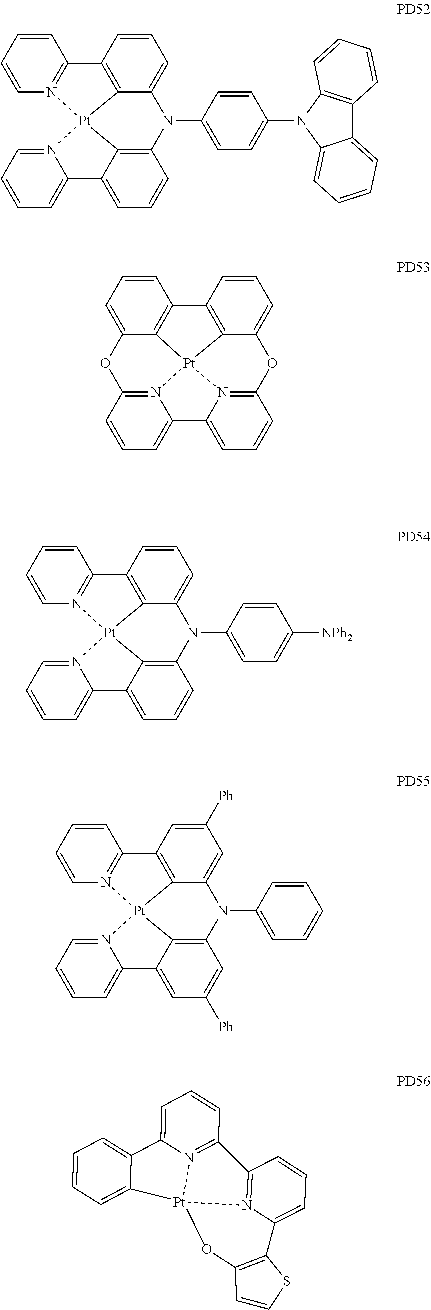

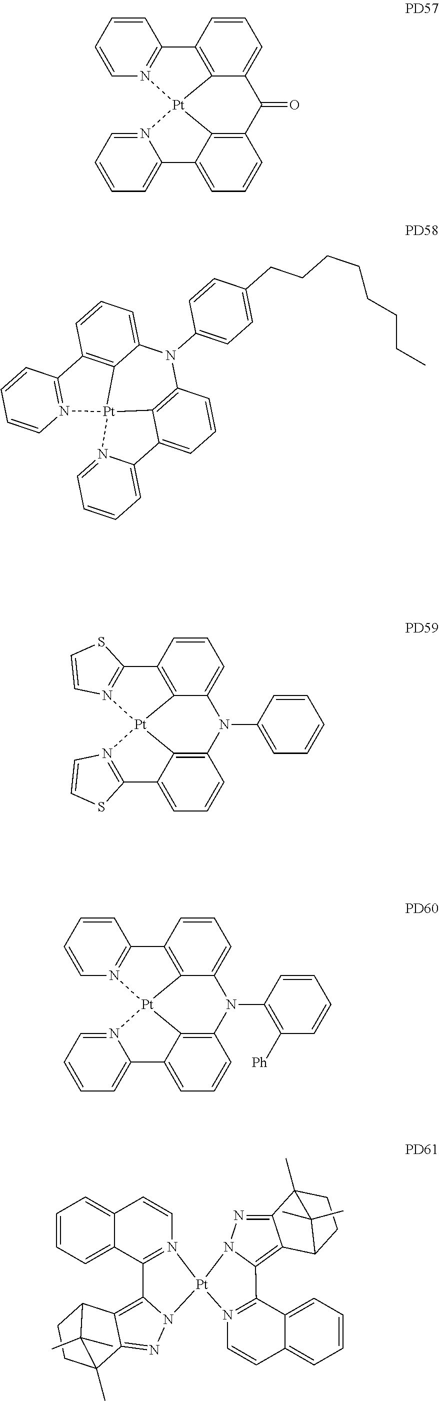

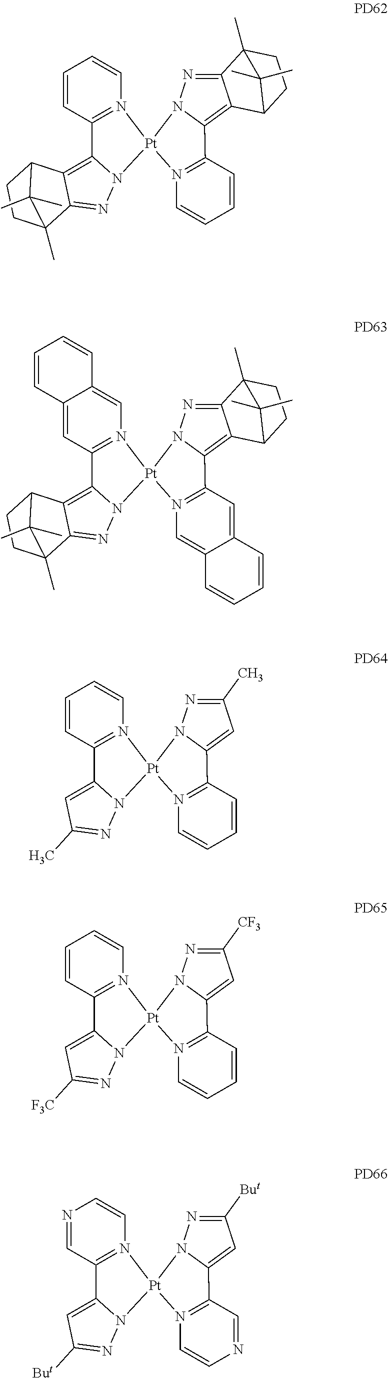

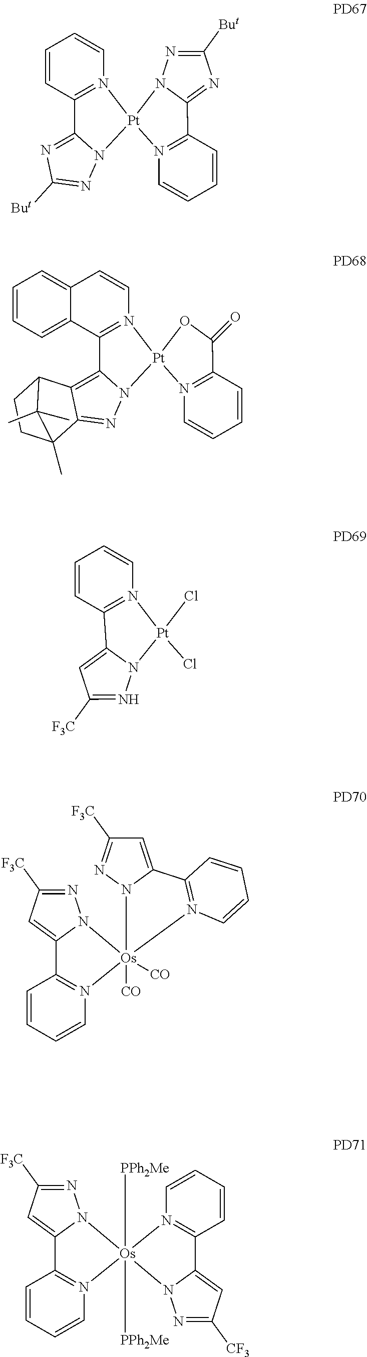

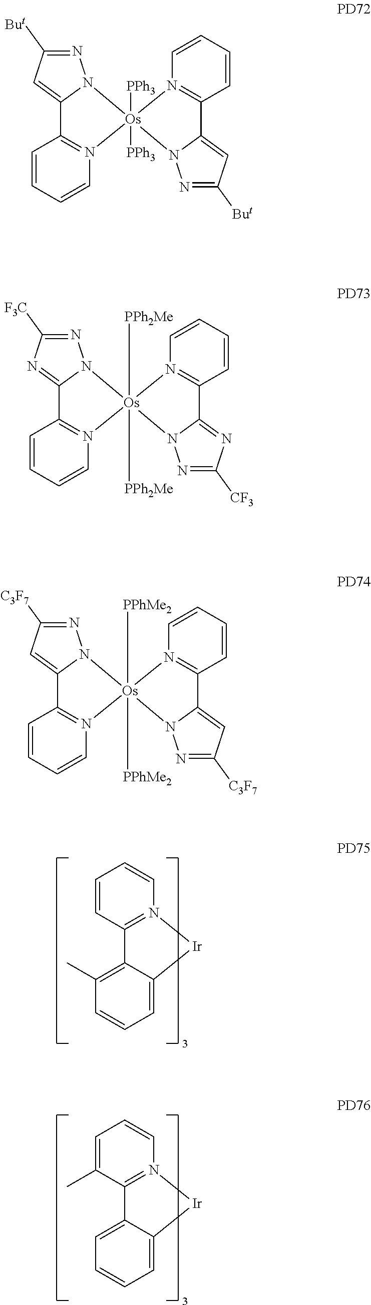

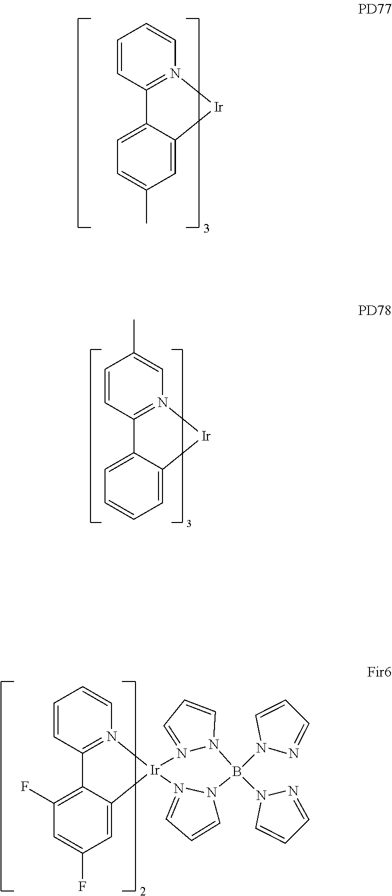

The expression “(an organic layer) includes at least one heterocyclic compound” used herein may include a case in which “(an organic layer) includes identical compounds represented by Formula 1” and a case in which “(an organic layer) includes two or more different heterocyclic compounds represented by Formula 1.”

For example, the organic layer may include, as the heterocyclic compound, only Compound 1. In this regard, Compound 1 may exist in an emission layer of the organic light-emitting device. In an embodiment, the organic layer may include, as the heterocyclic compound, Compound 1 and Compound 2. In this regard, Compound 1 and Compound 2 may exist in an identical layer (for example, Compound 1 and Compound 2 may all exist in an emission layer), or different layers (for example, Compound 1 may exist in an emission layer and Compound 2 may exist in a hole blocking layer).

The first electrode may be an anode, which is a hole injection electrode, and the second electrode may be a cathode, which is an electron injection electrode; or the first electrode may be a cathode, which is an electron injection electrode, and the second electrode may be an anode, which is a hole injection electrode.

The term “organic layer” as used herein refers to a single layer and/or a plurality of layers between the first electrode and the second electrode of the organic light-emitting device. The “organic layer” may include, in addition to an organic compound, an organometallic complex including metal.

FIGURE is a schematic view of an organic light-emitting device 10 according to an embodiment. Hereinafter, the structure of an organic light-emitting device according to an embodiment and a method of manufacturing an organic light-emitting device according to an embodiment will be described in connection with the FIGURE. The organic light-emitting device 10 includes a first electrode 11, an organic layer 15, and a second electrode 19, which are sequentially stacked.

A substrate may be additionally disposed under the first electrode 11 or above the second electrode 19. For use as the substrate, any substrate that is used in general organic light-emitting devices may be used, and the substrate may be a glass substrate or a transparent plastic substrate, each having excellent mechanical strength, thermal stability, transparency, surface smoothness, ease of handling, and water resistance.

In an embodiment, the first electrode 11 may be formed by depositing or sputtering a material for forming the first electrode 11 on the substrate. The first electrode 11 may be an anode. The material for forming the first electrode 11 may be a material with a high work function to facilitate hole injection. The first electrode 11 may be a reflective electrode, a semi-transmissive electrode, or a transmissive electrode. The material for forming the first electrode 11 may be indium tin oxide (ITO), indium zinc oxide (IZO), tin oxide (SnO₂), or zinc oxide (ZnO). In an embodiment, the material for forming the first electrode 11 may be metal or an alloy, such as magnesium (Mg), aluminum (Al), aluminum-lithium (Al—Li), calcium (Ca), magnesium-indium (Mg—In), or magnesium-silver (Mg—Ag).

The first electrode 11 may have a single-layered structure or a multi-layered structure including two or more layers. For example, the first electrode 11 may have a three-layered structure of ITO/Ag/ITO, but the structure of the first electrode 110 is not limited thereto.

The organic layer 15 is disposed on the first electrode 11.

The organic layer 15 may include a hole transport region, an emission layer, and an electron transport region.

The hole transport region may be disposed between the first electrode 11 and the emission layer.

The hole transport region may include at least one a hole injection layer, a hole transport layer, an electron blocking layer, or a buffer layer.

The hole transport region may include only either a hole injection layer or a hole transport layer. In an embodiment, the hole transport region may have a hole injection layer/hole transport layer structure or a hole injection layer/hole transport layer/electron blocking layer structure, which are sequentially stacked in this stated order from the first electrode 11.

When the hole transport region includes a hole injection layer (HIL), the hole injection layer may be formed on the first electrode 11 by using one or more suitable methods, for example, vacuum deposition, spin coating, casting, and/or Langmuir-Blodgett (LB) deposition.

When a hole injection layer is formed by vacuum deposition, the deposition conditions may vary according to a material that is used to form the hole injection layer, and the structure and thermal characteristics of the hole injection layer. For example, the deposition conditions may include a deposition temperature of about 100° C. to about 500° C., a vacuum pressure of about 10⁻⁸ torr to about 10⁻³ torr, and a deposition rate of about 0.01 Å/sec to about 100 Å/sec. However, the deposition conditions are not limited thereto.

When the hole injection layer is formed using spin coating, coating conditions may vary according to the material used to form the hole injection layer, and the structure and thermal properties of the hole injection layer. For example, a coating speed may be from about 2,000 rpm to about 5,000 rpm, and a temperature at which a heat treatment is performed to remove a solvent after coating may be from about 80° C. to about 200° C. However, the coating conditions are not limited thereto.

Conditions for forming a hole transport layer and an electron blocking layer may be understood by referring to conditions for forming the hole injection layer.

The hole transport region may include at least one m-MTDATA, TDATA, 2-TNATA, NPB, p-NPB, TPD, Spiro-TPD, Spiro-NPB, methylated-NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), a compound represented by Formula 201 below, or a compound represented by Formula 202 below:

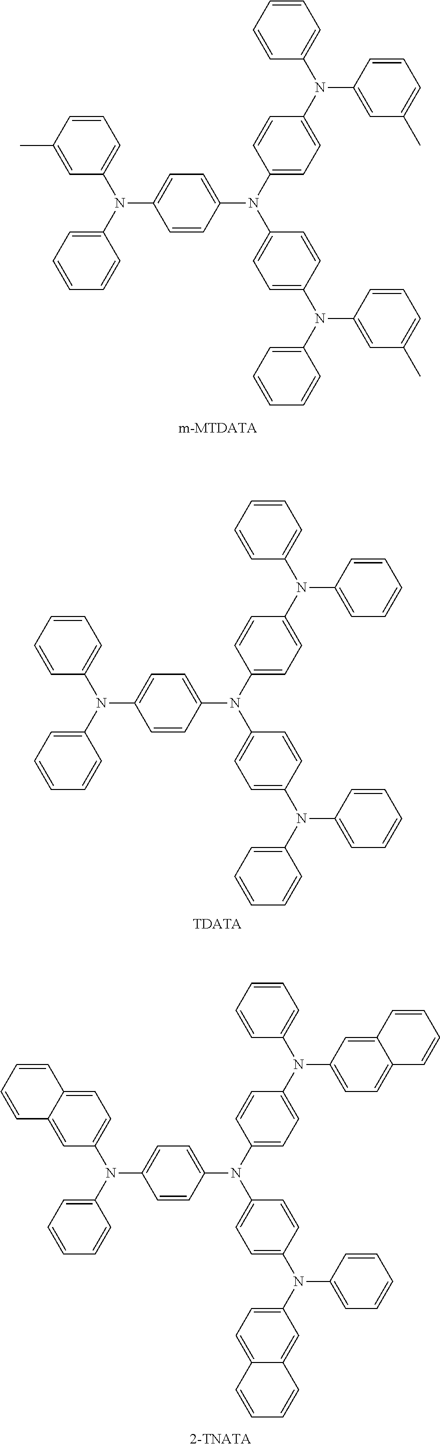

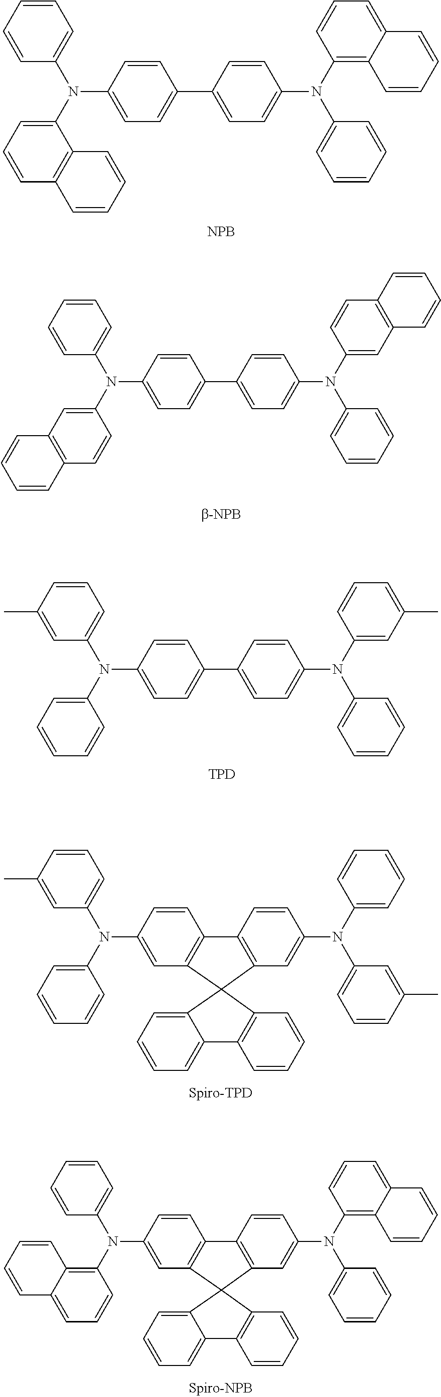

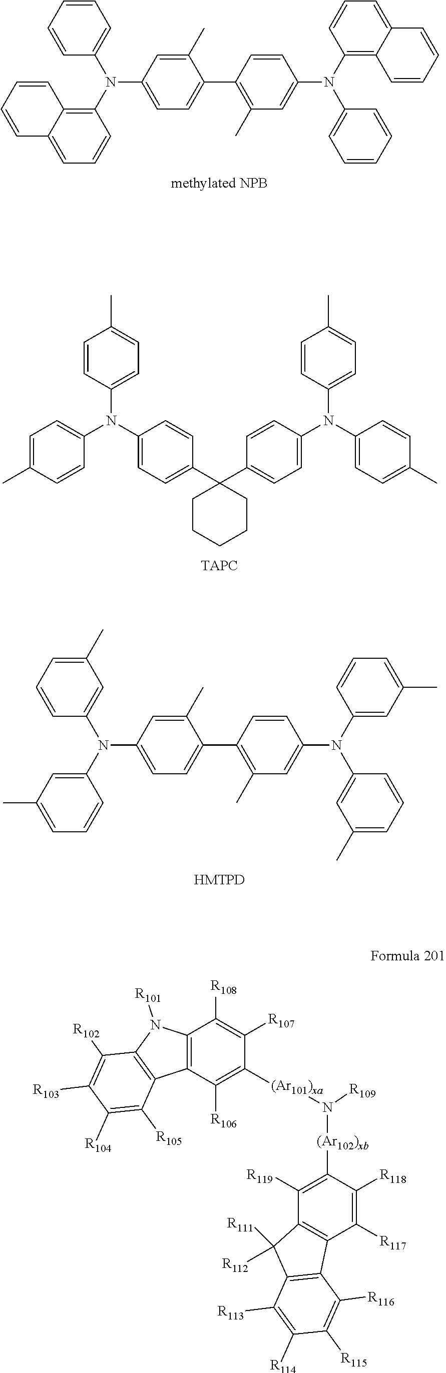

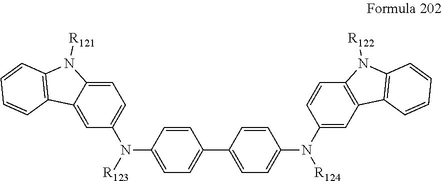

In Formula 201, Ar₁₀₁ and Ar₁₀₂ may each independently be:
a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, or a pentacenylene group; or
a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, or a pentacenylene group, each substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₂-C₁₀ heterocycloalkyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group.
xa and xb in Formula 201 may each independently be an integer from 0 to 5, or 0, 1 or 2. For example, xa may be 1 and xb may be 0, but xa and xb are not limited thereto.

In Formulae 201 and 202, R₁₀₁ to R₁₀₈, R₁₁₁ to R₁₁₉, and R₁₂₁ to R₁₂₄ may each independently be:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (for example, a methyl group, an ethyl group, a propyl group, a butyl group, pentyl group, a hexyl group, or the like), or a C₁-C₁₀ alkoxy group (for example, a methoxy group, an ethoxy group, a propoxy group, a butoxy group, a pentoxy group, or the like);
a C₁-C₁₀ alkyl group or a C₁-C₁₀ alkoxy group, each substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof or a phosphoric acid group or a salt thereof;
a phenyl group, a naphthyl group, an anthracenyl group, a fluorenyl group, or a pyrenyl group; or a phenyl group, a naphthyl group, an anthracenyl group, a fluorenyl group, or a pyrenyl group, each substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, or a C₁-C₁₀ alkoxy group,
but embodiments of the present disclosure are not limited thereto.

In Formula 201, R₁₀₉ may be:
a phenyl group, a naphthyl group, an anthracenyl group, or a pyridinyl group; or
a phenyl group, a naphthyl group, an anthracenyl group, or a pyridinyl group, each substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an anthracenyl group, or a pyridinyl group.

According to an embodiment, the compound represented by Formula 201 may be represented by Formula 201A below, but embodiments of the present disclosure are not limited thereto:

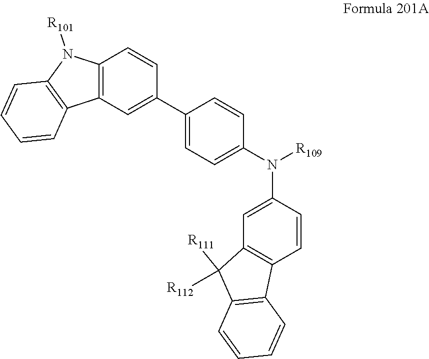

R₁₀₁, R₁₁₁, R₁₁₂, and R₁₀₉ in Formula 201A are the same as described above. For example, the compound represented by Formula 201, and the compound represented by Formula 202 may include compounds HT1 to HT20 illustrated below, but are not limited thereto:

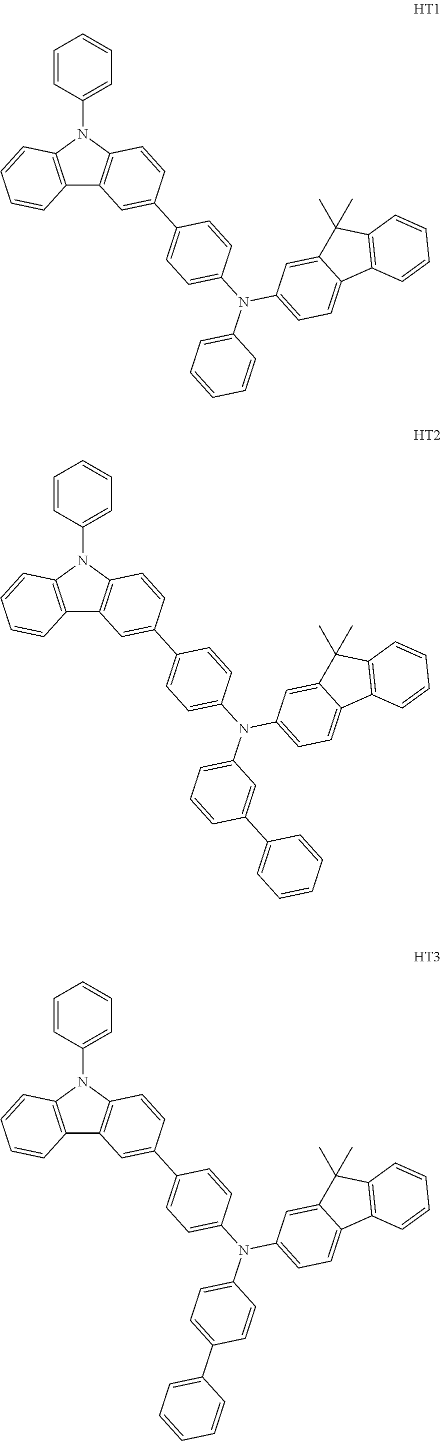 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

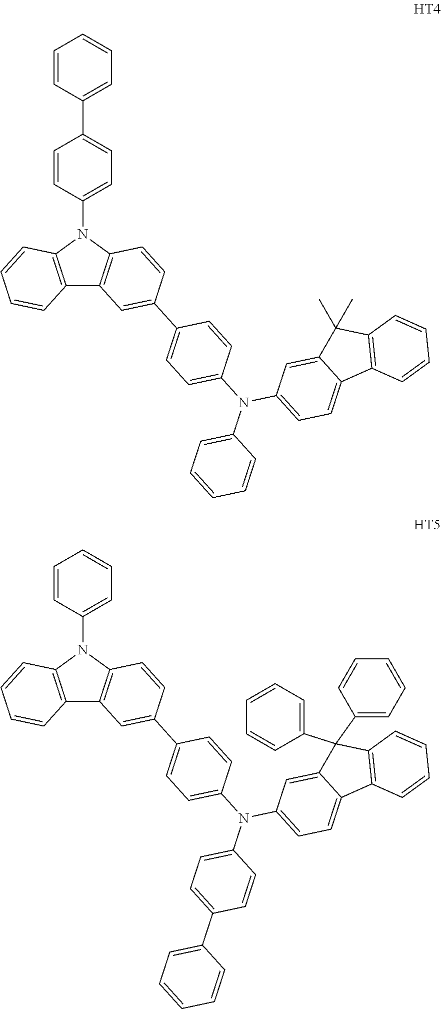 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

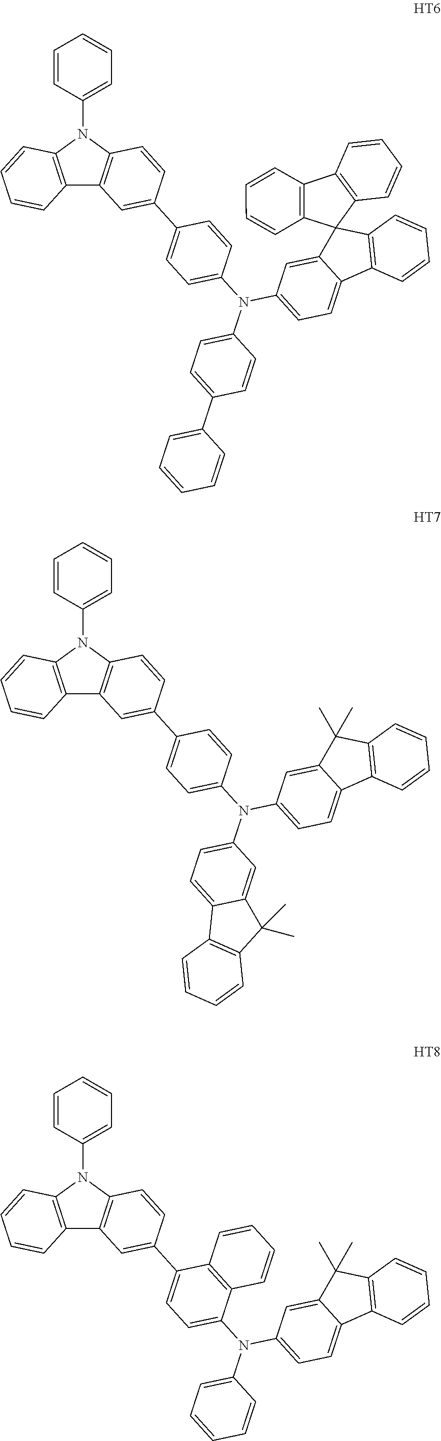 [SMILES: c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccccc3)c3ccc(-c4ccc5c(c4)c4ccccc4n5-c4ccccc4)c4ccccc34)cc21]

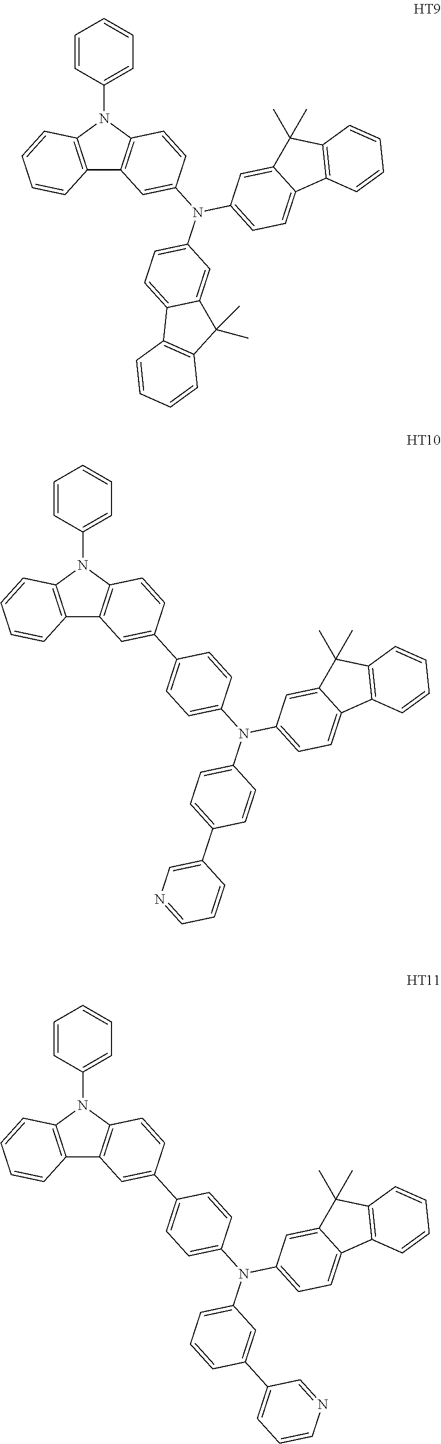 [SMILES: CC1(C)c2ccccc2-c2ccc(N(c3ccc4c(c3)C(C)(C)c3ccccc3-4)c3ccc4c(c3)c3ccccc3n4-c3ccccc3)cc21 ; CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21]

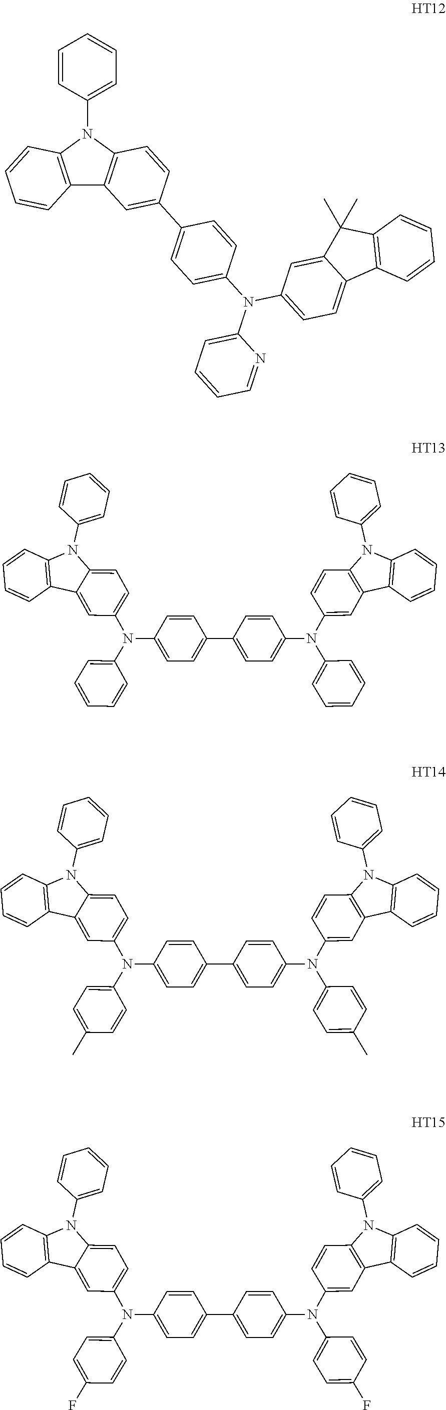 [SMILES: CC1(C)c2ccccc2-c2ccc(N(c3ccc(-c4ccc5c(c4)c4ccccc4n5-c4ccccc4)cc3)c3ccccn3)cc21 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

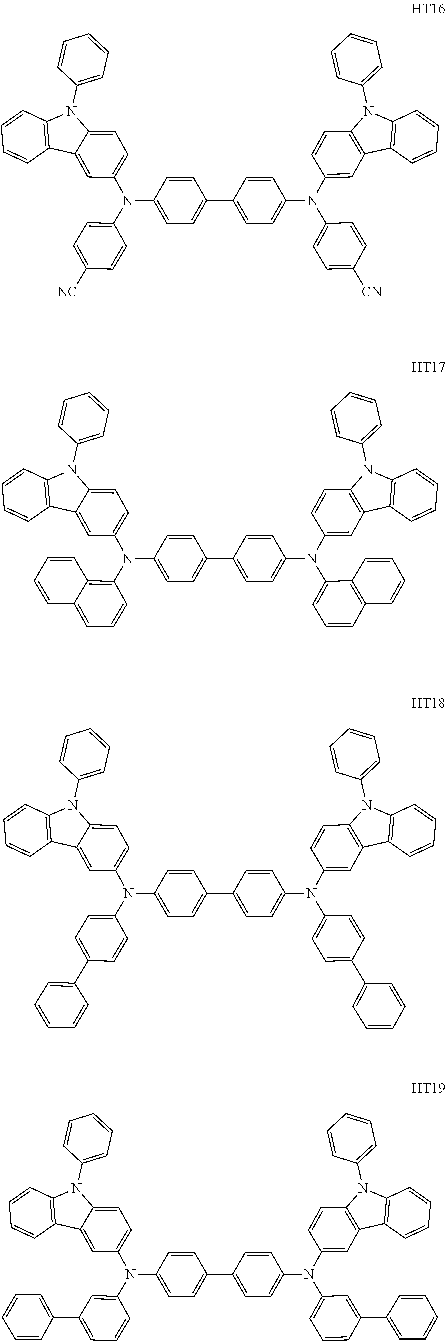 [SMILES: N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1 ; c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

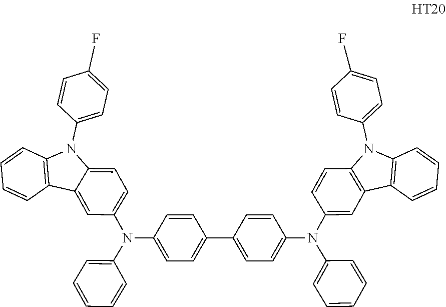 [SMILES: Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be in a range of about 100 Å to about 10,000 Å, for example, about 100 Å to about 1,000 Å. When the hole transport region includes at least one of a hole injection layer and a hole transport layer, a thickness of the hole injection layer may be in a range of about 100 Å to about 10,000 Å, for example, about 100 Å to about 1,000 Å, and a thickness of the hole transport layer may be in a range of about 50 Å to about 2,000 Å, for example about 100 Å to about 1,500 Å. When the thicknesses of the hole transport region, the hole injection layer, and the hole transport layer are within these ranges, satisfactory hole transporting characteristics may be obtained without a substantial increase in driving voltage.

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant. The p-dopant may be a quinone derivative, a metal oxide, or a cyano group-containing compound, but embodiments of the present disclosure are not limited thereto. Non-limiting examples of the p-dopant are a quinone derivative, such as tetracyanoquinodimethane (TCNQ) or 2,3,5,6-tetrafluoro-tetracyano-1,4-benzoquinodimethane (F4-TCNQ); a metal oxide, such as a tungsten oxide or a molybdenum oxide; and a cyano group-containing compound, such as Compound HT-D1 or Compound HT-D2 below, but are not limited thereto:

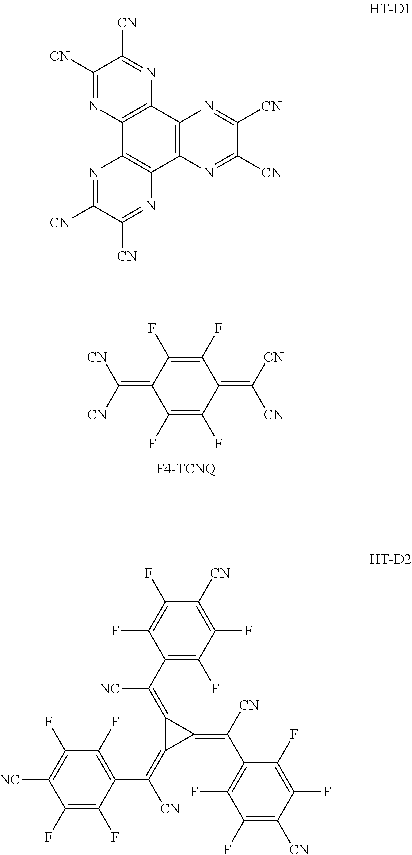

The hole transport region may further include a buffer layer.

Also, the buffer layer may compensate for an optical resonance distance according to a wavelength of light emitted from the emission layer, and thus, efficiency of a formed organic light-emitting device may be improved.

Then, an emission layer may be formed on the hole transport region by vacuum deposition, spin coating, casting, LB deposition, or the like. When the emission layer is formed by vacuum deposition or spin coating, the deposition or coating conditions may be similar to those applied in forming the hole injection layer although the deposition or coating conditions may vary according to a compound that is used to form the emission layer.

The electron transport region may further include an electron blocking layer. The electron blocking layer may include, for example, mCP, but a material therefor is not limited thereto:

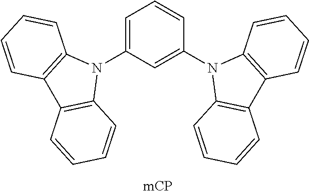 [SMILES: c1cc(-n2c3ccccc3c3ccccc32)cc(-n2c3ccccc3c3ccccc32)c1]

A thickness of the electron blocking layer may be in a range of about 50 Å to about 1,000 Å, for example, about 70 Å to about 500 Å. When the thickness of the electron blocking layer is within the range described above, the electron blocking layer may have satisfactory electron blocking characteristics without a substantial increase in driving voltage.

When the organic light-emitting device is a full-color organic light-emitting device, the emission layer may be patterned into a red emission layer, a green emission layer, and a blue emission layer. In an embodiment, due to a stacked structure including a red emission layer, a green emission layer, and/or a blue emission layer, the emission layer may emit white light.

The emission layer may include the heterocyclic compound represented by Formula 1.

For example, the emission layer may include the heterocyclic compound represented by Formula 1 alone.

In an embodiment, the emission layer may include the heterocyclic compound represented by Formula 1, and may further include:
i) the second compound (for example, a compound represented by Formula H-1);
ii) the organometallic compound represented by Formula 81; or
iii) any combination thereof.

The heterocyclic compound represented by Formula 1, the second compound, and the organometallic compound represented by Formula 81 are the same as described above.

In an embodiment, when the emission layer includes a host and a dopant, an amount of the dopant may be in a range of about 0.01 parts to about 20 parts by weight based on 100 parts by weight of the host, but embodiments of the present disclosure are not limited thereto. When the amount of the dopant is satisfied within the range above, the emission without extinction phenomenon may be realized.

In an embodiment, when the emission layer includes the heterocyclic compound represented by Formula 1 and the second compound, a weight ratio of the heterocyclic compound represented by Formula 1 to the second compound may be in a range of about 1:99 to about 99:1, for example, about 70:30 to about 30:70. In an embodiment, a weight ratio of the heterocyclic compound represented by Formula 1 to the second compound may be in a range of about 60:40 to about 40:60. When the weight ratio of the heterocyclic compound represented by Formula 1 to the second compound is satisfied within the range above, the charge transport balance in the emission layer may be efficiently achieved.

A thickness of the emission layer may be in a range of about 100 Å to about 1,000 Å, for example, about 200 Å to about 600 Å. When the thickness of the emission layer is within this range, excellent light-emission characteristics may be obtained without a substantial increase in driving voltage.

Then, an electron transport region may be disposed on the emission layer.

The electron transport region may include at least one of a hole blocking layer, an electron transport layer, or an electron injection layer.

For example, the electron transport region may have a hole blocking layer/electron transport layer/electron injection layer structure or an electron transport layer/electron injection layer structure, but the structure of the electron transport region is not limited thereto. The electron transport layer may have a single-layered structure or a multi-layered structure including two or more different materials.

Conditions for forming the hole blocking layer, the electron transport layer, and the electron injection layer which constitute the electron transport region may be understood by referring to the conditions for forming the hole injection layer.

When the electron transport region includes a hole blocking layer, the hole blocking layer may include, for example, at least one of BCP or Bphen, but may also include other materials:

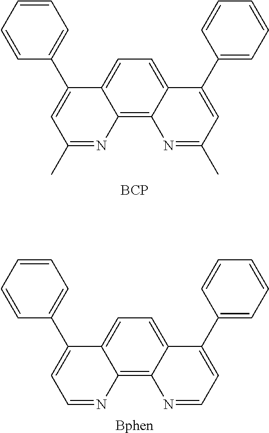

The hole blocking layer may include the heterocyclic compound represented by Formula 1.

A thickness of the hole blocking layer may be in a range of about 20 Å to about 1,000 Å, for example, about 30 Å to about 300 Å. When the thickness of the hole blocking layer is within these ranges, the hole blocking layer may have excellent hole blocking characteristics without a substantial increase in driving voltage.

The electron transport layer may further include at least one of BCP, Bphen, Alq₃, BAlq, TAZ, or NTAZ:

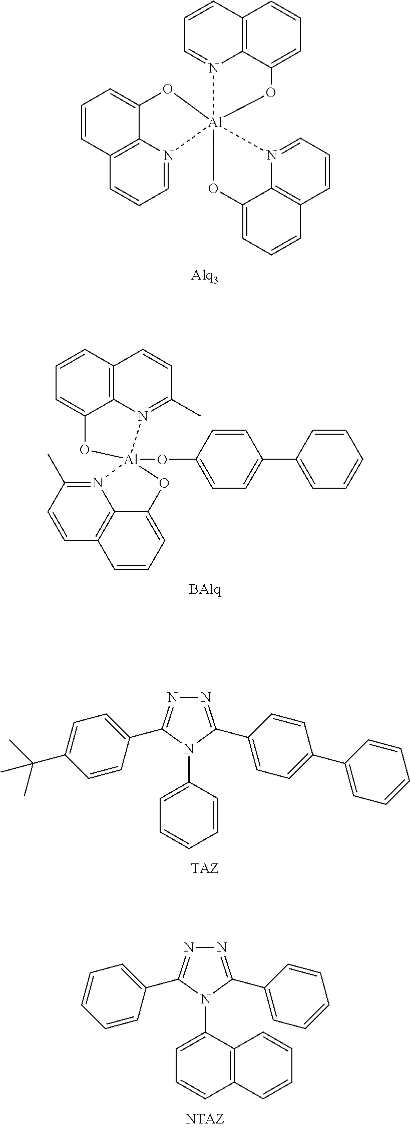

In an embodiment, the electron transport layer may include at least one of Compounds ET1, ET2, or ET3, but embodiments of the present disclosure are not limited thereto:

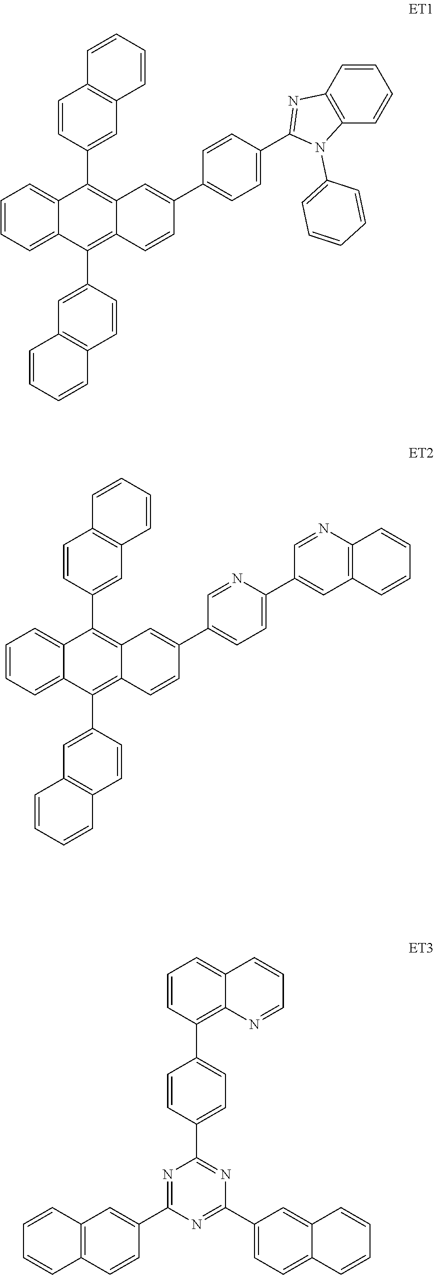 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc2cc(-c3nc(-c4ccc(-c5cccc6cccnc56)cc4)nc(-c4ccc5ccccc5c4)n3)ccc2c1]

A thickness of the electron transport layer may be in a range of about 100 Å to about 1,000 Å, for example, about 150 Å to about 500 Å. When the thickness of the electron transport layer is within the range described above, the electron transport layer may have satisfactory electron transport characteristics without a substantial increase in driving voltage.

Also, the electron transport layer may further include, in addition to the materials described above, a metal-containing material.

The metal-containing material may include a Li complex. The Li complex may include, for example, Compound ET-D1 (lithium quinolate, LiQ) or ET-D2.

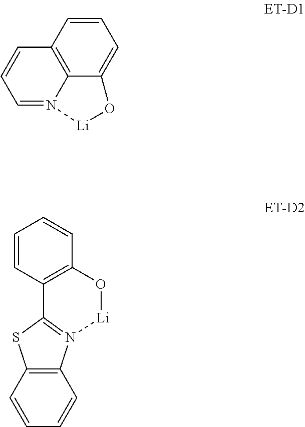

The electron transport region may include an electron injection layer that promotes flow of electrons from the second electrode 19 thereinto.

The electron injection layer may include at least one of LiQ, LiF, NaCl, CsF, Li₂O, or BaO.

A thickness of the electron injection layer may be in a range of about 1 Å to about 100 Å, and, for example, about 3 Å to about 90 Å. When the thickness of the electron injection layer is within the range described above, the electron injection layer may have satisfactory electron injection characteristics without a substantial increase in driving voltage.

The second electrode 19 is disposed on the organic layer 15. The second electrode 19 may be a cathode. A material for forming the second electrode 19 may be metal, an alloy, an electrically conductive compound, or a combination thereof, which have a relatively low work function. For example, lithium (Li), magnesium (Mg), aluminum (Al), aluminum-lithium (Al—Li), calcium (Ca), magnesium-indium (Mg—In), or magnesium-silver (Mg—Ag) may be formed as the material for forming the second electrode 19. To manufacture a top-emission type light-emitting device, a transmissive electrode formed using ITO or IZO may be used as the second electrode 19.

Hereinbefore, the organic light-emitting device has been described with reference to the FIGURE, but embodiments of the present disclosure are not limited thereto.

The term “C₁-C₆₀ alkyl group” as used herein refers to a linear or branched aliphatic saturated hydrocarbon monovalent group having 1 to 60 carbon atoms, and examples thereof include a methyl group, an ethyl group, a propyl group, an isobutyl group, a sec-butyl group, a tert-butyl group, a pentyl group, an isoamyl group, and a hexyl group. The term “C₁-C₆₀ alkylene group” as used herein refers to a divalent group having the same structure as the C₁-C₆₀ alkyl group.

The term “C₁-C₆₀ alkoxy group” as used herein refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₂₀-C₆₀ alkenyl group” as used herein refers to a hydrocarbon group having at least one double bond in the middle or at the terminus of the C₂₀-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group having at least one triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon monocyclic group having 3 to 10 carbon atoms, and non-limiting examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₂-C₁₀ heterocycloalkyl group” as used herein refers to a monovalent saturated monocyclic group having at least one heteroatom of N, O, P, Si, Se, Ge, or S as a ring-forming atom and 1 to 10 carbon atoms, and non-limiting examples thereof include a tetrahydrofuranyl group, and a tetrahydrothiophenyl group. The term “C₂-C₁₀ heterocycloalkylene group” as used herein refers to a divalent group having the same structure as the C₂-C₁₀ heterocycloalkyl group.

The term “C₃-C₁₀ cycloalkenyl group” as used herein refers to a monovalent monocyclic group that has 3 to 10 carbon atoms and at least one double bond in the ring thereof and no aromaticity, and non-limiting examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₂-C₁₀ heterocycloalkenyl group” as used herein refers to a monovalent monocyclic group that has at least one heteroatom of N, O, P, Si, Se, Ge, or S as a ring-forming atom, 1 to 10 carbon atoms, and at least one double bond in its ring. Examples of the C₂-C₁₀ heterocycloalkenyl group are a 2,3-dihydrofuranyl group, and a 2,3-dihydrothiophenyl group. The term “C₂-C₁₀ heterocycloalkenylene group” as used herein refers to a divalent group having the same structure as the C₂-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₁-C₆₀ heteroaryl group” as used herein refers to a monovalent group having a heterocyclic aromatic system that has at least one heteroatom of N, O, P, Si, Ge, or S as a ring-forming atom, and 1 to 60 carbon atoms. The term C₁-C₆₀ heteroarylene group” as used herein refers to a divalent group having a heterocyclic aromatic system that has at least one heteroatom N, O, P, Si, Ge, or S as a ring-forming atom, and 1 to 60 carbon atoms. Non-limiting examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each include two or more rings, the rings may be fused to each other.

The term “C₆-C₆₀ aryloxy group” as used herein refers to —OA₁₀₂ (wherein A₁₀₂ is the C₆-C₆₀ aryl group), and the term “C₆-C₆₀ arylthio group” as used herein refers to —SA₁₀₃ (wherein A₁₀₃ is the C₆-C₆₀ aryl group).

The term “C₅-C₃₀ carbocyclic group” as used herein refers to a monovalent, divalent, or higher valency group containing only carbon atoms in the ring(s) thereof, which may be saturated, unsaturated, or aromatic, having 5 to 30 carbon atoms.

Detailed examples of the C₅-C₃₀ carbocyclic group are a cyclopentyl group, a cyclohexenyl group, a decalinyl group, a phenyl group, a naphthyl group, an anthracenyl group, a phenanthrenyl group, a pyrenyl group, and a chrysenyl group. When the C₅-C₃₀ carbocyclic group includes two or more rings, the rings may be condensed to each other or linked via a bond.

The term “C₂-C₃₀ heterocarbocyclic group” as used herein refers to a monovalent, divalent or higher valency group having one or more rings, at least two carbon atoms and at least one heteroatom of N, O, P, Si, Se, Ge, or S as a ring-forming atom in the ring(s) thereof, which may be saturated, unsaturated, or aromatic, having 2 to 30 carbon atoms. Detailed examples of the C₂-C₃₀ heterocarbocyclic group are a furanyl group, a morpholinyl group, a piperidinyl group, a piperazinyl group, a pyridinyl group, a pyrimidinyl group, a pyrazinyl group, a pyridazinyl group, a triazinyl group, a quinolinyl group, and an isoquinolinyl group. When the C₂-C₃₀ heterocarbocyclic group includes two or more rings, the rings may be condensed to each other.

The term “monovalent non-aromatic condensed polycyclic group” as used herein refers to a monovalent group having two or more rings condensed to each other, only carbon atoms (for example, the number of carbon atoms may be in a range of 8 to 60) as a ring-forming atom, and no aromaticity in its entire molecular structure. Non-limiting examples of the monovalent non-aromatic condensed polycyclic group include a fluorenyl group. The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group having two or more rings condensed to each other, a heteroatom of N, O, P, Si, Se, Ge, or S, other than carbon atoms (for example, the number of carbon atoms may be in a range of 2 to 60), as a ring-forming atom, and no aromaticity in its entire molecular structure. Non-limiting examples of the monovalent non-aromatic condensed heteropolycyclic group include a carbazolyl group. The term “divalent non-aromatic condensed heteropolycyclic group” as used herein refers to a divalent group having the same structure as the monovalent non-aromatic condensed heteropolycyclic group.

In the present specification, at least one substituent of the substituted C₁-C₆₀ alkyl group, the substituted C₂-C₆₀ alkenyl group, the substituted C₂-C₆₀ alkynyl group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, or the substituted monovalent non-aromatic condensed heteropolycyclic group may be:
deuterium, —CD₃, —CD₂H, —CDH₂, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with at least one of deuterium, —CD₃, —CD₂H, —CDH₂, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₄)(Q₁₅), or —B(Q₁₆)(Q₁₇),
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group;
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each substituted with at least one of deuterium, —CD₃, —CD₂H, —CDH₂, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₄)(Q₂₅), or —B(Q₂₆)(Q₂₇), or
—Si(Q₃₁)(Q₃₂)(Q₃₃), —N(Q₃₄)(Q₃₅), or —B(Q₃₆)(Q₃₇), and
Q₁₁ to Q₁₇, Q₂₁ to Q₂₇, and Q₃₁ to Q₃₇ may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group.

* and *′ as used herein, unless defined otherwise, each refer to a binding site to a neighboring atom in a corresponding formula.

Hereinafter, a compound and an organic light-emitting device according to embodiments are described in detail with reference to Synthesis Example and Examples. However, the organic light-emitting device is not limited thereto. The wording ‘B’ was used instead of ‘A’ used in describing Synthesis Examples means that a molar equivalent of ‘A’ was identical to a molar equivalent of ‘B’.

### EXAMPLES

### Synthesis Example 1: Synthesis of Compound 1

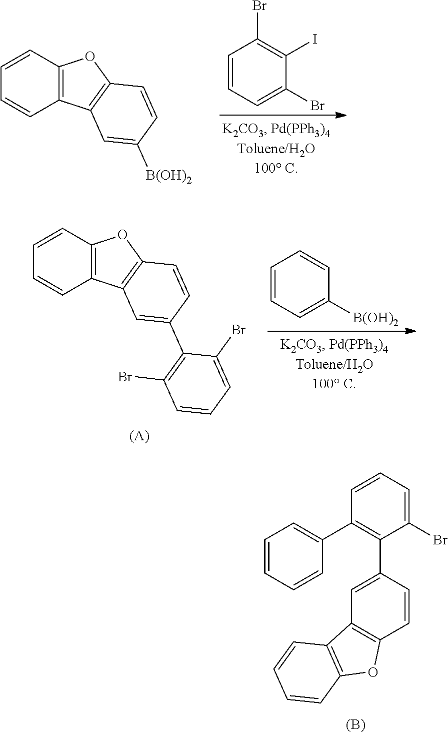

1) Synthesis of Intermediate (A)

Dibenzo[b,d]furan-2-ylboronic acid (15.0 g, 70.75 mmol), 1,3-dibromo-2-iodobenzene (25.6 g, 70.75 mmol), tetrakis(triphenylphosphine)palladium(0) (4.07 g, 3.53 mmol), and potassium carbonate (29.3 g, 212.25 mmol) were dissolved in 280 ml of toluene and 70 ml of water and stirred at a temperature of 100° C. for 12 hours. After the reaction was completed, the reaction mixture was cooled to room temperature, and an organic layer was extracted therefrom by using saturated ammonium chloride (NH₄Cl) and dichloromethane (DCM). The extracted organic layer was dried by using anhydrous magnesium sulfate (MgSO₄), filtered, and then concentrated under reduced pressure. Then, silica gel column chromatography was performed thereon to obtain Intermediate (A) (22.75 g, yield=80%).

LC-Mass (calcd.: 399.91 g/mol, found: M+1=401 g/mol).

(2) Synthesis of Intermediate (B)

Intermediate (A) (22.70 g, 56.46 mmol), phenylboronic acid (6.88 g, 56.46 mmol), tetrakis(triphenylphosphine)palladium(0) (3.26 g, 2.82 mmol), and potassium carbonate (23.44 g, 169.8 mmol) were dissolved in 240 ml of toluene and 60 ml of water and stirred at a temperature of 100° C. for 6 hours. After the reaction was completed, the reaction mixture was cooled to room temperature, and an organic layer was extracted therefrom by using saturated ammonium chloride (NH₄Cl) and DCM. The extracted organic layer was dried by using anhydrous magnesium sulfate (MgSO₄), filtered, and then concentrated under reduced pressure. Then, silica gel column chromatography was performed thereon to obtain Intermediate (B) (11.27 g, yield=50%).

LC-Mass (calcd.: 398.03 g/mol, found: M+1=399 g/mol).

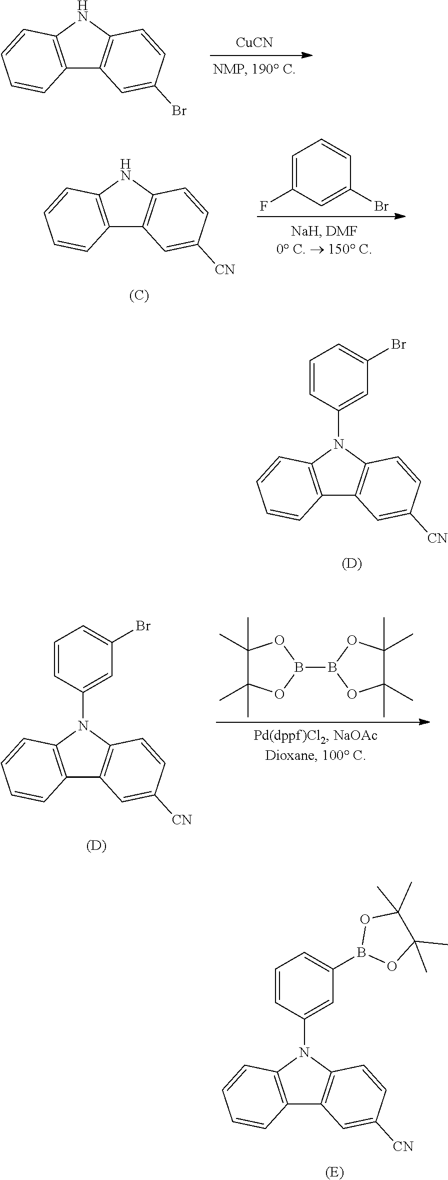

(3) Synthesis of Intermediate (C)

3-bromo-9H-carbazole (10.0 g, 40.63 mmol) and copper (I) cyanide (3.82 g, 42.66 mmol) were dissolved in 220 ml of N-methyl-2-pyrrolidone (NMP) and stirred at a temperature of 190° C. for 6 hours. After the reaction was completed, the reaction mixture was cooled to room temperature, and an organic layer was extracted therefrom by using saturated ammonium chloride (NH₄Cl) and ethyl acetate (EA). The extracted organic layer was dried by using anhydrous magnesium sulfate (MgSO₄), filtered, and concentrated under reduced pressure. Then silica gel column chromatography was performed thereon to obtain Intermediate (C) (5.31 g, yield=68%).

LC-Mass (calcd.: 192.07 g/mol, found: M+1=193 g/mol).

(4) Synthesis of Intermediate (D)

Intermediate (C) (5.31 g, 27.58 mmol) was dissolved in 200 ml of N,N-dimethylformamide (DMF) and cooled to a temperature 0° C. Sodium hydride (NaH, 60% dispersion in mineral oil) (1.16 g, 28.96 mmol) was slowly added to the solution and stirred at a temperature of 0° C. for 30 minutes. 1-bromo-3-fluorobenzene (5.3 g, 30.34 mmol) was dissolved in 15 ml of DMF, and this solution was slowly added to the solution of Intermediate (C) for 10 minutes. The mixture was heated to a temperature of 150° C. and stirred for 18 hours. After the reaction was completed, the reaction mixture was cooled to room temperature, and an organic layer was extracted therefrom by using saturated ammonium chloride (NH₄Cl) and EA. The extracted organic layer was dried by using anhydrous magnesium sulfate (MgSO₄), filtered, and then concentrated under pressure. Then, silica gel column chromatography was performed thereon to obtain Intermediate (D) (7.46 g, yield=78%).

LC-Mass (calcd.: 346.01 g/mol, found: M+1=347 g/mol).

(5) Synthesis of Intermediate (E)

Intermediate (D) (7.46 g, 21.51 mmol), bis(pinacolato)diboron (6.0 g, 23.66 mmol), [1,1′-Bis(diphenylphosphino)ferrocene]dichloropalladium(II) (786 mg, 1.08 mmol), and sodium acetate (5.29 g, 64.53 mmol) were dissolved in dioxane and stirred at a temperature of 100° C. for 12 hours. After the reaction was completed, the reaction mixture was cooled to room temperature, an organic layer was extracted therefrom by using saturated ammonium chloride (NH₄Cl) and DCM. The extracted organic layer was dried by using anhydrous magnesium sulfate (MgSO₄), filtered, and then concentrated under reduced pressure. Then, silica gel column chromatography was performed thereon to obtain Intermediate (E) (5.26 g, yield=62%).

LC-Mass (calcd.: 394.19 g/mol, found: M+1=395 g/mol).

(6) Synthesis of Compound 1

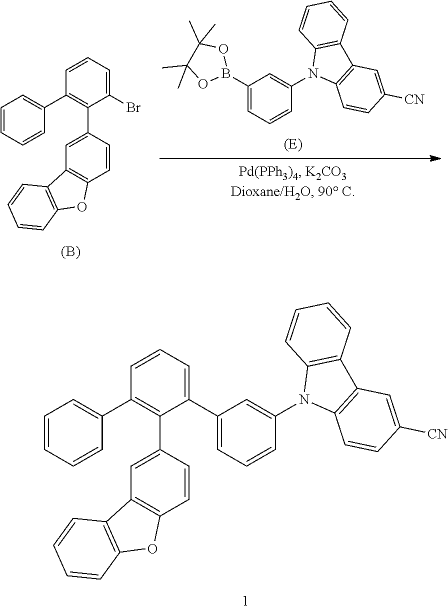

Intermediate (B) (4.43 g, 11.11 mmol), Intermediate (E) (5.26 g, 13.33 mmol), tetrakis(triphenylphosphine)palladium(0) (1.28 g, 1.11 mmol), and potassium carbonate (3.07 g, 22.22 mmol) were dissolved in 80 ml of dioxane and 20 ml of water and stirred at a temperature of 90° C. for 5 hours. After the reaction was completed, the reaction mixture was cooled to room temperature, and an organic layer was extracted therefrom by using saturated ammonium chloride (NH₄Cl) and DCM. The extracted organic layer was dried by using anhydrous magnesium sulfate (MgSO₄), filtered, and then concentrated under reduced pressure. Then, silica gel column chromatography was performed thereon to obtain Compound 1 (4.69 g, yield=72%).

LC-Mass (calcd.: 586.21 g/mol, found: M+1=587 g/mol).

### Synthesis Example 2: Synthesis of Compound 2

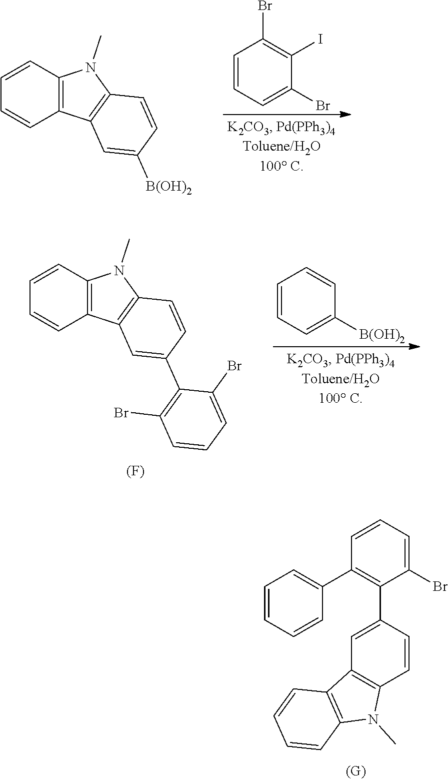 [SMILES: Cn1c2ccccc2c2cc(B(O)O)ccc21 ; Brc1cccc(Br)c1I ; Cn1c2ccccc2c2cc(-c3c(Br)cccc3Br)ccc21 ; OB(O)c1ccccc1 ; "(F)" ; Cn1c2ccccc2c2cc(-c3c(Br)cccc3-c3ccccc3)ccc21]

(1) Synthesis of Intermediate (F)

Intermediate (F) (13.84 g, yield=75%) was obtained in the same manner as in Synthesis of Intermediate (A), except that (9-methyl-9H-carbazol-3-yl)boronic acid (10.0 g, 44.44 mmol) was used instead of dibenzo[b,d]furan-2-ylboronic acid during synthesis.

LC-Mass (calcd.: 412.94 g/mol, found: M+1=414 g/mol).

(2) Synthesis of Intermediate (G)

Intermediate (G) (6.18 g, yield=45%) was obtained in the same manner as in Synthesis of Intermediate (B), except that Intermediate (F) (13.84 g, 33.33 mmol) was used instead of Intermediate (A) during synthesis.

LC-Mass (calcd.: 411.06 g/mol, found: M+1=412 g/mol)

(3) Synthesis of Compound 2

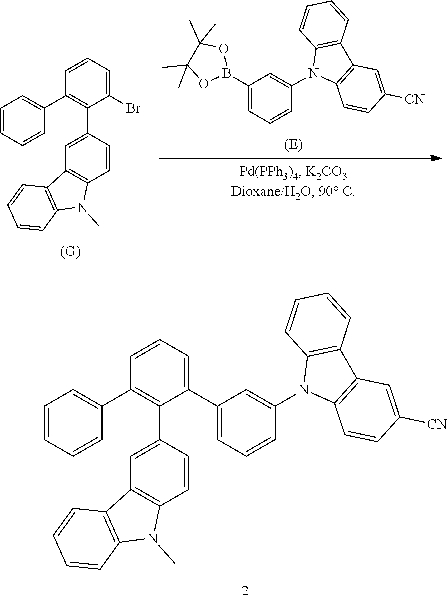

Compound 2 (4.75 g, yield=72%) was obtained in the same manner as in Synthesis of Compound 1, except that Intermediate (G) (4.54 g, 11.00 mmol) was used instead of Intermediate (B) during synthesis.

LC-Mass (calcd.: 599.24 g/mol, found: M+1=600 g/mol).

### Synthesis Example 3: Synthesis of Compound 3

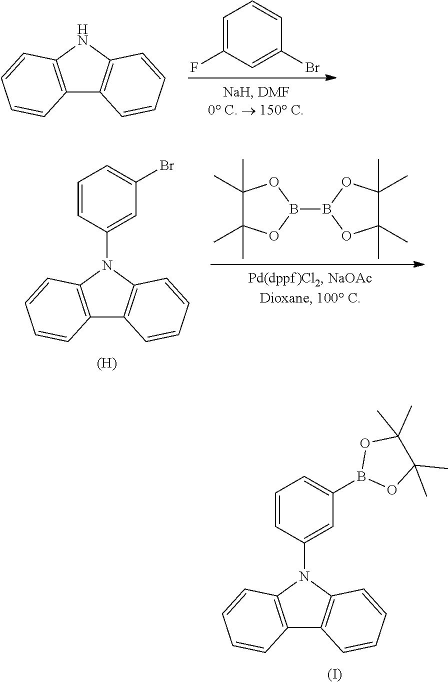

(1) Synthesis of Intermediate (H)

Intermediate (H) (12.64 g, yield=82%) was obtained in the same manner as in the same manner as in Synthesis of Intermediate (D), except that carbazole (8.00 g, 47.85 mmol) was used instead of Intermediate (C) during synthesis.

LC-Mass (calcd.: 321.02 g/mol, found: M+1=322 g/mol).

(2) Synthesis of Intermediate (I)

Intermediate (I) (10.14 g, yield=70%) was obtained in the same manner as in Synthesis of Intermediate (E), except that Intermediate (H) (12.64 g, 39.24 mmol) was used instead of Intermediate (D) during synthesis.

LC-Mass (calcd.: 369.19 g/mol, found: M+1=370 g/mol).

(3) Synthesis of Compound 3

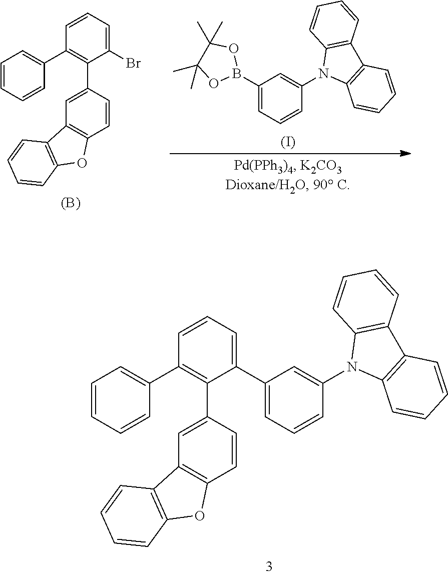

Compound 3 (8.29 g, yield=75%) was obtained in the same manner as in Synthesis of Compound 1, except that Intermediate (I) (8.00 g, 21.67 mmol) was used instead of Intermediate (E) during synthesis.

LC-Mass (calcd.: 561.21 g/mol, found: M+1=562 g/mol).

### Synthesis Example 4: Synthesis of Compound 4

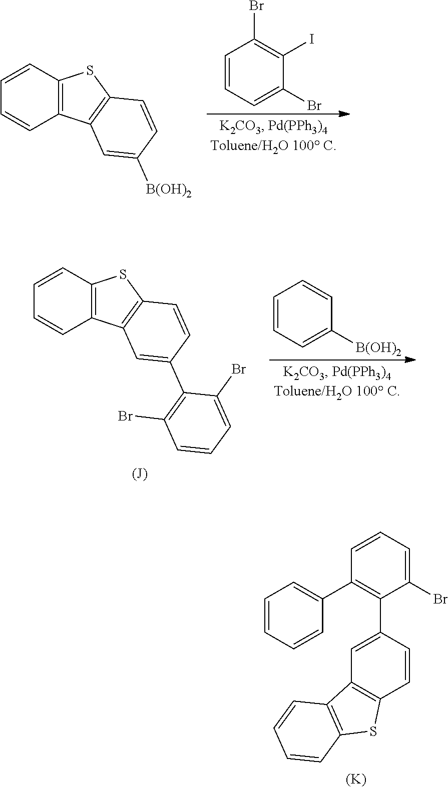

(1) Synthesis of Intermediate (J)

Intermediate (J) (12.83 g, yield=70%) was obtained in the same manner as in Synthesis of Intermediate (A), except that dibenzo[b,d]thiophen-2-ylboronic acid (10.0 g, 43.84 mmol) was used instead of dibenzo[b,d]furan-2-ylboronic acid during synthesis.

LC-Mass (calcd.: 415.89 g/mol, found: M+1=416 g/mol).

(2) Synthesis of Intermediate (K)

Intermediate (K) (5.74 g, yield=45%) was obtained in the same manner as in Synthesis of Intermediate (B), except that Intermediate (J) (12.83 g, 30.69 mmol) was used instead of Intermediate (A) during synthesis.

LC-Mass (calcd.: 414.00 g/mol, found: M+1=415 g/mol).

(3) Synthesis of Compound 4

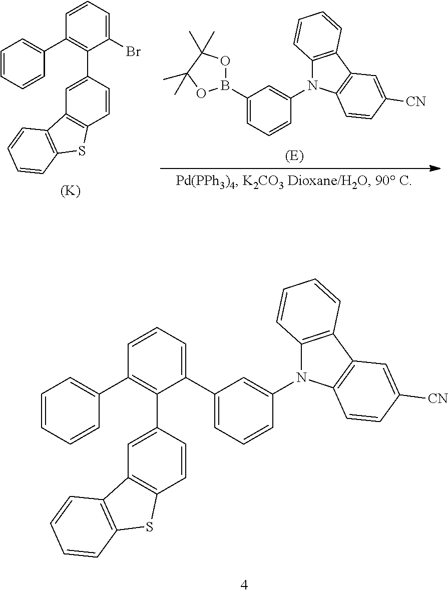

Compound 4 (6.08 g, yield=73%) was obtained in the same manner as in Synthesis of Compound 1, except that Intermediate (K) (5.74 g, 13.81 mmol) was used instead of Intermediate (B) during synthesis.

LC-Mass (calcd.: 602.18 g/mol, found: M+1=603 g/mol).

### Synthesis Example 5: Synthesis of Compound 5

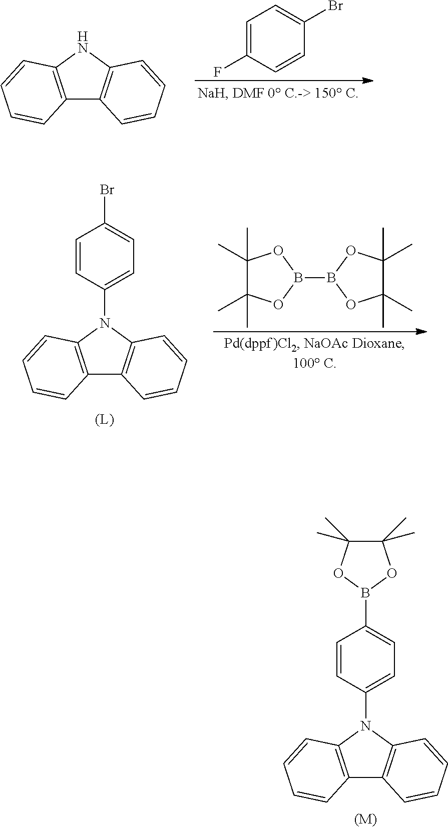

(1) Synthesis of Intermediate (L)

Intermediate (L) (7.36 g, yield=80%) was obtained in the same manner as in Synthesis of Intermediate (H), except that 1-bromo-4-fluorobenzene (5.00 g, 28.57 mmol) was used instead of 1-bromo-3-fluorobenzene during synthesis.

LC-Mass (calcd.: 321.02 g/mol, found: M+1=322 g/mol).

(2) Synthesis of Intermediate (M)

Intermediate (M) (5.91 g, yield=70%) was obtained in the same manner as in Synthesis of Intermediate (E), except that Intermediate (L) (7.36 g, 22.86 mmol) was used instead of Intermediate (D).

LC-Mass (calcd.: 369.19 g/mol, found: M+1=370 g/mol).

(3) Synthesis of Compound 5

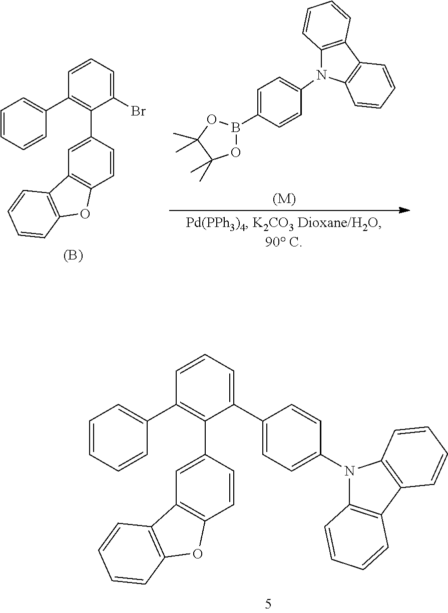

Compound 5 (5.96 g, yield=73%) was obtained in the same manner as in Synthesis of Compound 1, except that Intermediate (M) (5.91 g, 16.00 mmol) was used instead of Intermediate (E) during synthesis.

LC-Mass (calcd.: 561.21 g/mol, found: M+1=562 g/mol).

### Synthesis Example 6: Synthesis of Compound 7

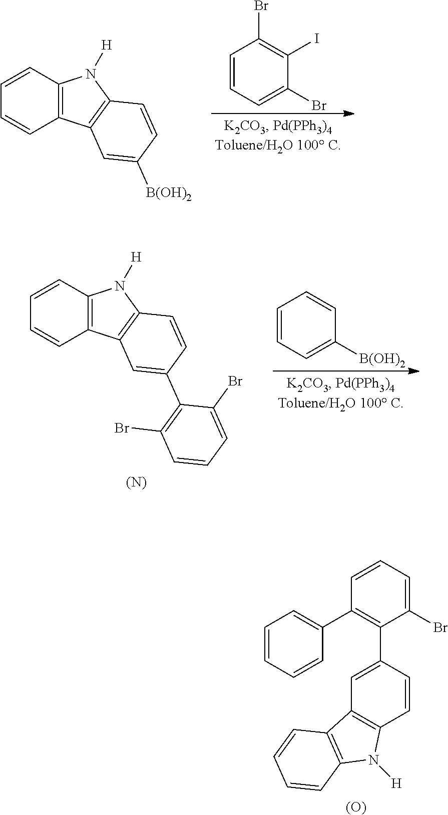

(1) Synthesis of Intermediate (N)

Intermediate (N) (13.31 g, yield=70%) was obtained in the same manner as in Synthesis of Intermediate (A), except that (9H-carbazol-3-yl)boronic acid (10.0 g, 47.39 mmol) was used instead of dibenzo[b,d]furan-2-ylboronic acid during synthesis.

LC-Mass (calcd.: 398.92 g/mol, found: M+1=400 g/mol).

(2) Synthesis of Intermediate (O)

Intermediate (O) (5.29 g, yield=40%) was obtained in the same manner as in Synthesis of Intermediate (B), except that Intermediate (N) (13.31 g, 33.17 mmol) was used instead of Intermediate (A) during synthesis.

LC-Mass (calcd.: 397.05 g/mol, found: M+1=398 g/mol).

(3) Synthesis of Compound 7

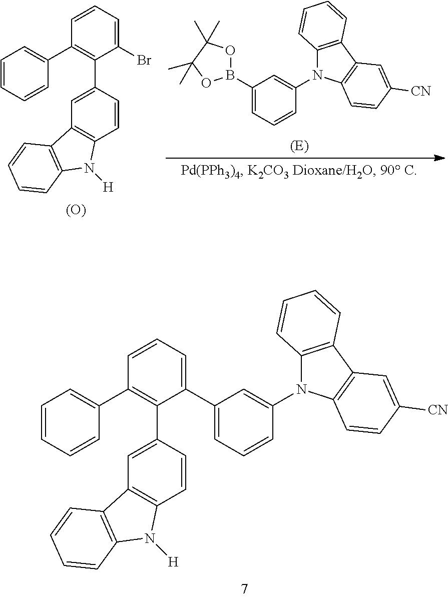

Compound 7 (5.44 g, yield=70%) was obtained in the same manner as in Synthesis of Compound 1, except that Intermediate (O) (5.29 g, 13.27 mmol) was used instead of Intermediate (B) during synthesis.

LC-Mass (calcd.: 585.22 g/mol, found: M+1=586 g/mol).

### Synthesis Example 7: Synthesis of Compound 10

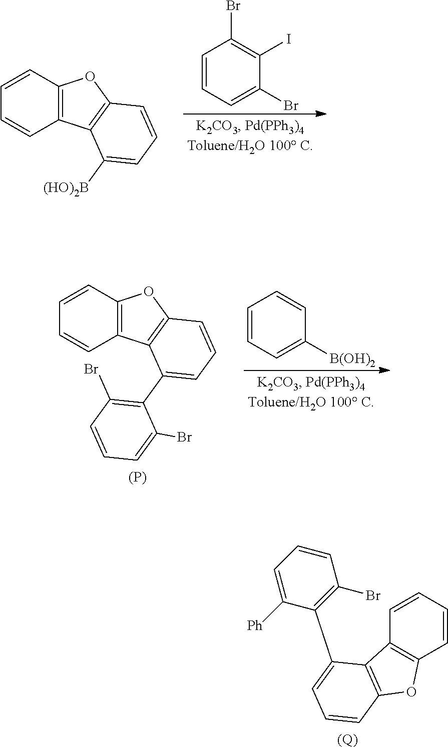

(1) Synthesis of Intermediate (P)

Intermediate (P) (13.47 g, yield=71%) was obtained in the same manner as in Synthesis of Intermediate (A), except that dibenzo[b,d]furan-1-ylboronic acid (10.0 g, 47.17 mmol) was used instead of dibenzo[b,d]furan-2-ylboronic acid during synthesis.

LC-Mass (calcd.: 399.91 g/mol, found: M+1=401 g/mol).

(2) Synthesis of Intermediate (Q)

Intermediate (Q) (5.62 g, yield=42%) was obtained in the same manner as in Synthesis of Intermediate (B), except that Intermediate (P) (13.47 g, 33.49 mmol) was used instead of Intermediate (A) during synthesis.

LC-Mass (calcd.: 398.03 g/mol, found: M+1=399 g/mol).

(3) Synthesis of Compound 10

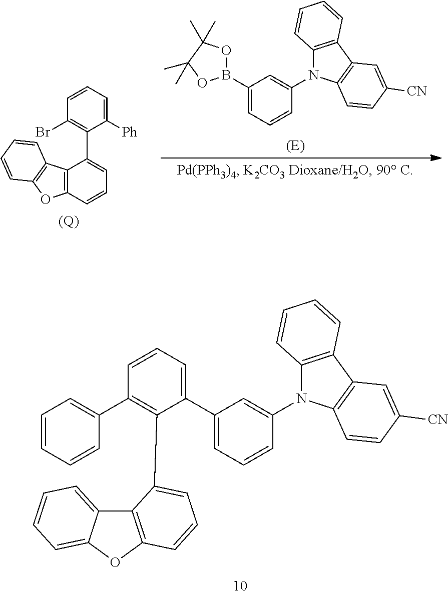

Compound 10 (6.03 g, yield=73%) was obtained in the same manner as in Synthesis of Compound 1, except that Intermediate (Q) (5.62 g, 14.07 mmol) was used instead of Intermediate (B) during synthesis.

LC-Mass (calcd.: 586.21 g/mol, found: M+1=587 g/mol).

### Synthesis Example 8: Synthesis of Compound 13

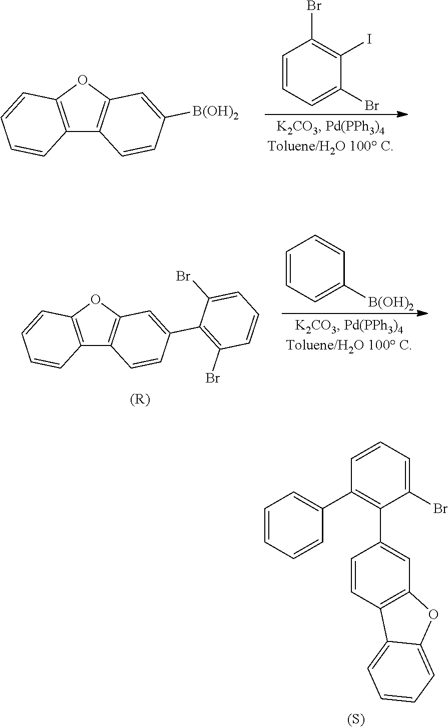

(1) Synthesis of Intermediate (R)

Intermediate (R) (13.85 g, yield=73%) was obtained in the same manner as in Synthesis of Intermediate (A), except that dibenzo[b,d]furan-3-ylboronic acid (10.0 g, 47.17 mmol) was used instead of dibenzo[b,d]furan-2-ylboronic acid during synthesis.

LC-Mass (calcd.: 399.91 g/mol, found: M+1=401 g/mol).

(2) Synthesis of Intermediate (S)

Intermediate (S) (6.19 g, yield=45%) was obtained in the same manner as in Synthesis of Intermediate (B), except that Intermediate (R) (13.85 g, 34.43 mmol) was used instead of Intermediate (A) during synthesis.

LC-Mass (calcd.: 398.03 g/mol, found: M+1=399 g/mol).

(3) Synthesis of Compound 13

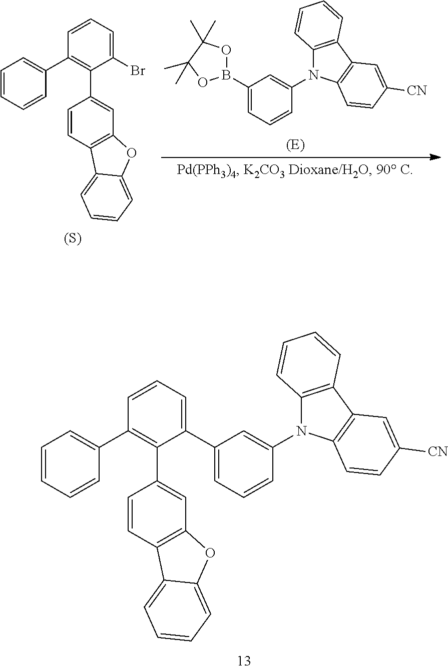

Compound 13 (6.82 g, yield=75%) was obtained in the same manner as in Synthesis of Compound 1, except that Intermediate (S) (6.19 g, 15.49 mmol) was used instead of Intermediate (B) during synthesis.

LC-Mass (calcd.: 586.21 g/mol, found: M+1=587 g/mol).

### Synthesis Example 9: Synthesis of Compound 14

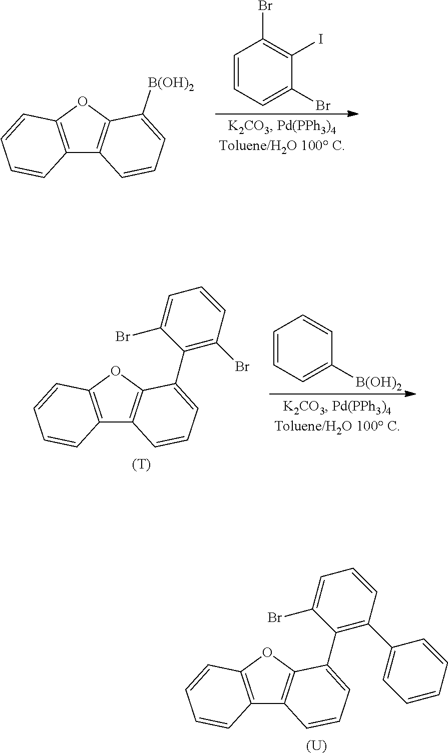

(1) Synthesis of Intermediate (T)

Intermediate (13.28 g, yield=70%) was synthesized in the same manner as in Synthesis of Intermediate (A), except that dibenzo[b,d]furan-4-ylboronic acid (10.0 g, 47.17 mmol) was used instead of dibenzo[b,d]furan-2-ylboronic acid during synthesis.

LC-Mass (calcd.: 399.91 g/mol, found: M+1=401 g/mol).

(2) Synthesis of Intermediate (U)

Intermediate (U) (6.06 g, yield=46%) was obtained in the same manner as in Synthesis of Intermediate (B), except that Intermediate (T) (13.28 g, 33.02 mmol) was used instead of Intermediate (A) during synthesis.

LC-Mass (calcd.: 398.03 g/mol, found: M+1=399 g/mol).

(3) Synthesis of Compound 14

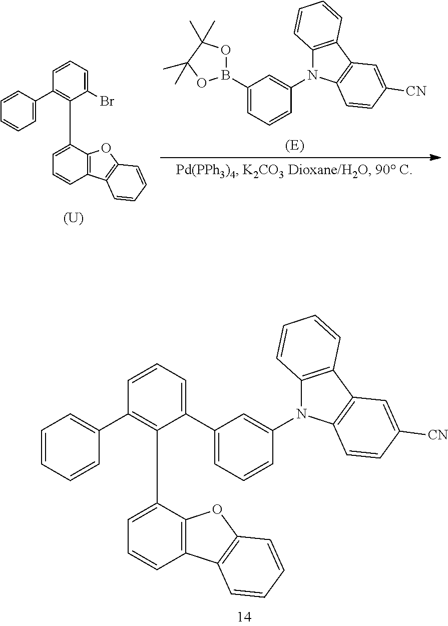

Compound 14 (6.51 g, yield=73%) was obtained in the same manner as in Synthesis of Compound 1, except that Intermediate (U) (6.06 g, 15.19 mmol) was used instead of Intermediate (B) during synthesis.

LC-Mass (calcd.: 586.21 g/mol, found: M+1=587 g/mol).

### Synthesis Example 10: Synthesis of Compound 15

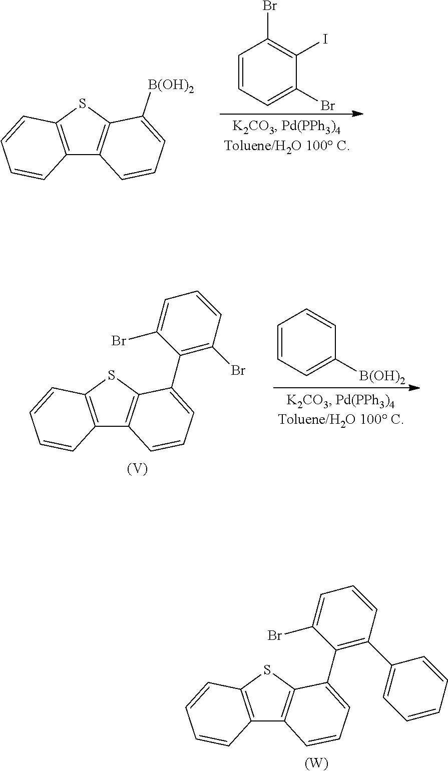

(1) Synthesis of Intermediate (V)

Intermediate (V) (12.83 g, yield=70%) was obtained in the same manner as in Synthesis of Intermediate (A), except that dibenzo[b,d]thiophen-4-ylboronic acid (10.0 g, 43.85 mmol) was used instead of dibenzo[b,d]furan-2-ylboronic acid during synthesis.

LC-Mass (calcd.: 415.89 g/mol, found: M+1=417 g/mol).

(2) Synthesis of Intermediate (W)

Intermediate (W) (5.48 g, yield=43%) was obtained in the same manner as in Synthesis of Intermediate (B), except that Intermediate (V) (12.83 g, 30.70 mmol) was used instead of Intermediate (A) during synthesis.

LC-Mass (calcd.: 414.00 g/mol, found: M+1=415 g/mol).

(3) Synthesis of Compound 15

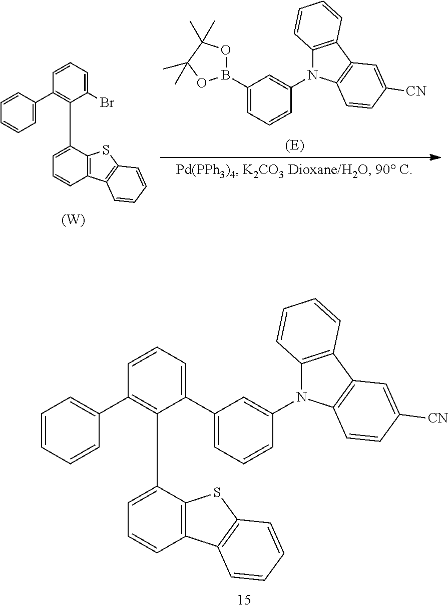

Compound 15 (5.57 g, yield=70%) was obtained in the same manner as in Synthesis of Compound 1, except that Intermediate (W) (5.48 g, 13.20 mmol) was used instead of Intermediate (B) during synthesis.

LC-Mass (calcd.: 602.18 g/mol, found: M+1=603 g/mol).

### Evaluation Example 1: Evaluation of Photoluminescence Quantum Yield (PLQY) and Thermal Characteristics

Samples in which a host H19 and a dopant (15 wt %) were co-deposited on a quartz cell to a thickness of 100 Å by using Compounds 1, 2, 3, 4, 5, and 10 and Comparative Compounds 1 and 2 as a dopant were manufactured by a co-deposition process. The samples were excited in a nitrogen atmosphere by excited light having a wavelength of 340 nm by using C9920-02 and PMA-11 (available from Hamamatsu photonics), and PLOY was measured. PLOY was expressed by a relative value (%) when PLOY of Comparative Compound 2 was 100%. Results thereof are shown in Table 2.

In addition, thermal analysis (N₂ atmosphere, temperature range: room temperature to 800° C. (10° C./min)-TGA, room temperature to 400° C.-DSC, pan type: Pt Pan in disposal Al pan (TGA), disposal Al pan (DSC)) was performed on Compounds 1, 2, 3, 4, 5, and 10 and Comparative Compounds 1 and 2 by using thermo gravimetric analysis (TGA) and differential scanning calorimetry (DSC), and results thereof are shown in Table 2 as relative values (based on Celsius temperature) for Comparative Compound 1.

TABLE 2

| | PLQY | Tg | Td |
|---|---|---|---|
| Compound | (relative value, %) | (relative value, %) | (relative value, %) |
| Compound 1 | 119 | 151 | 133 |
| Compound 2 | 110 | 153 | 136 |
| Compound 3 | 106 | 141 | 125 |
| Compound 4 | 118 | 154 | 138 |
| Compound 5 | 120 | 146 | 129 |
| Compound 10 | 113 | 146 | 130 |
| Comparative | 104 | 100 | 100 |
| Compound 1 | | | |
| Comparative | 100 | 123 | 113 |
| Compound 2 | | | |

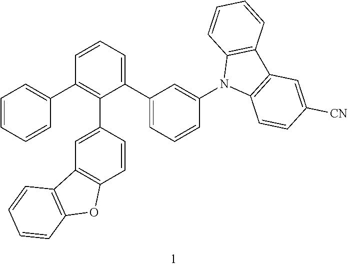 [SMILES: N#Cc1ccc2c(c1)c1ccccc1n2-c1cccc(-c2cccc(-c3ccccc3)c2-c2ccc3oc4ccccc4c3c2)c1]

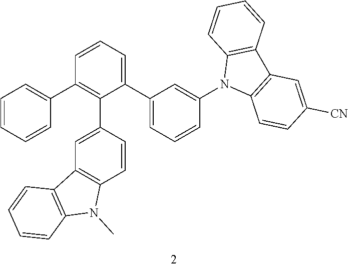 [SMILES: Cn1c2ccccc2c2cc(-c3c(-c4ccccc4)cccc3-c3cccc(-n4c5ccccc5c5cc(C#N)ccc54)c3)ccc21]

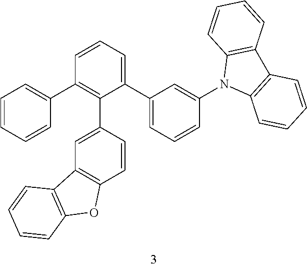 [SMILES: c1ccc(-c2cccc(-c3cccc(-n4c5ccccc5c5ccccc54)c3)c2-c2ccc3oc4ccccc4c3c2)cc1]

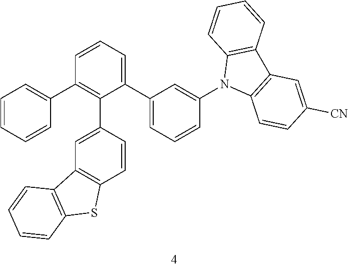 [SMILES: N#Cc1ccc2c(c1)c1ccccc1n2-c1cccc(-c2cccc(-c3ccccc3)c2-c2ccc3sc4ccccc4c3c2)c1]

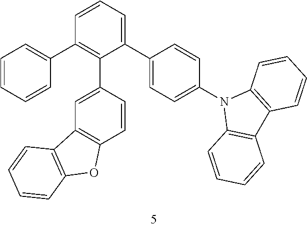 [SMILES: c1ccc(-c2cccc(-c3ccc(-n4c5ccccc5c5ccccc54)cc3)c2-c2ccc3oc4ccccc4c3c2)cc1]

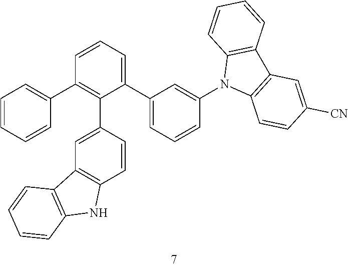 [SMILES: N#Cc1ccc2c(c1)c1ccccc1n2-c1cccc(-c2cccc(-c3ccccc3)c2-c2ccc3[nH]c4ccccc4c3c2)c1]

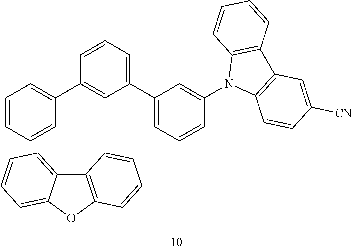 [SMILES: N#Cc1ccc2c(c1)c1ccccc1n2-c1cccc(-c2cccc(-c3ccccc3)c2-c2cccc3oc4ccccc4c23)c1]

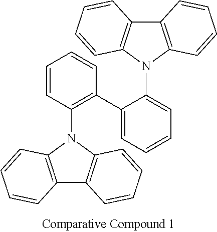 [SMILES: c1ccc(-n2c3ccccc3c3ccccc32)c(-c2ccccc2-n2c3ccccc3c3ccccc32)c1]

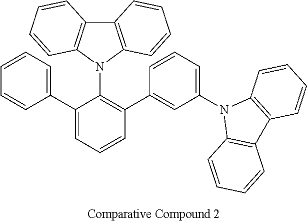 [SMILES: c1ccc(-c2cccc(-c3cccc(-n4c5ccccc5c5ccccc54)c3)c2-n2c3ccccc3c3ccccc32)cc1]

Referring to Table 2, it is confirmed that the heterocyclic compound has high PLQY and excellent thermal stability, as compared with those of Comparative Compounds 1 and 2.

Since the heterocyclic compound has excellent electric characteristics and thermal stability, an organic light-emitting device including the heterocyclic compound may have low driving voltage, high efficiency, high power, high quantum efficiency, and long lifespan characteristics.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope as defined by the following claims.

What is claimed is:

1. A heterocyclic compound represented by Formula 1: 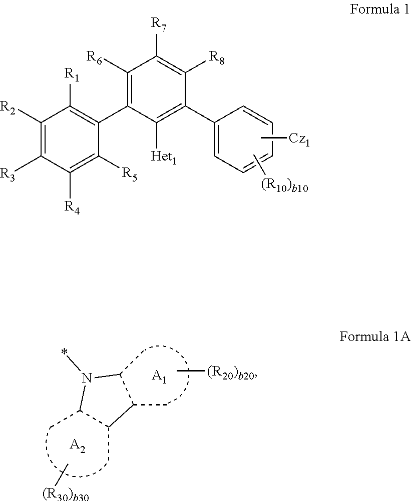
wherein, in Formula 1,
Cz₁ is a group represented by Formula 1A,
Het₁ is a group represented by Formulae 2B-1 to 2B-7: 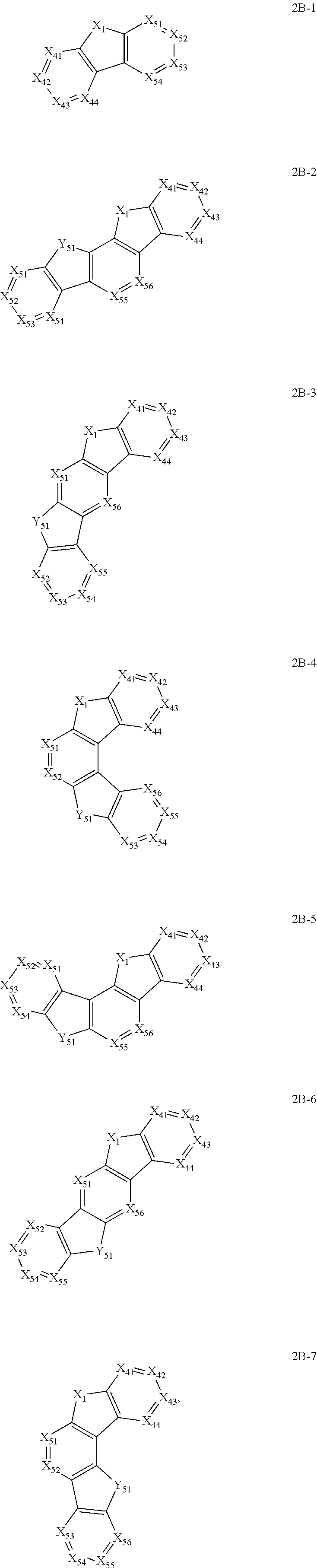
wherein, in Formulae 2B-1 to 2B-7,
X₁ is O, S, or N(R₉),
X₄₁ is N, C(R₄₁), or *—C, X₄₂ is N, C(R₄₂), or *—C, X₄₃ is N, C(R₄₃), or *—C, X₄₄ is N, C(R₄₄), or *—C, and one of X₄₁ to X₄₄ is *—C
X₅₁ is N or C(R₅₁), X₅₂ is N or C(R₅₂), X₅₃ is N or C(R₅₃), X₅₄ is N or C(R₅₄), X₅₅ is N or C(R₅₅), X₅ is N or C(R₅₆), and each of X₅₁ to X₅₆ is not N,
Y₅₁ is O, S, N(R₅₇), C(R₅₇)(R₅₈), or Si(R₅₇)(R₅₈),
and
* indicates a binding site to a neighboring atom,
in Formulae 1, 1A, and 2B-1 to 2B-7,
A₁ to A₄ are each independently a C₅-C₆₀ carbocyclic group or a C₁-C₆₀ heterocyclic group,
R₁ to R₁₀, R₂₀, R₃₀, R₄₁ to R₄₄, and R₅₁ to R₅₈ are each independently hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), or —B(Q₆)(Q₇),
b10 is an integer from 1 to 4,
b20, b30, b40, and b50 are each independently an integer from 1 to 10,
if Het₁ is a group represented by Formula 2B-1 and X₁ is O or S, then on e of X₄₁, X₄₂ and X₄₄ is *—C,
at least one substituent of the substituted C₅-C₆₀ carbocyclic group, the substituted C₁-C₆₀ heterocyclic group, the substituted C₆-C₆₀ arylene group, the substituted C₁-C₆₀ heteroarylene group, the substituted divalent non-aromatic condensed polycyclic group, the substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group is:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a mono valent non-aromatic condensed heteropolycyclic group, —Si(Q₁₁)(Q₁₂)(Q₁₃), —N(Q₁₄)(Q₁₅), or —B(Q₁₆)(Q₁₇);
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group;
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆ heteroaryl group, a monovalent non-aromatic condensed polycyclic group, or a monovalent non-aromatic condensed heteropolycyclic group, each substituted with at least one deuterium, —F, —Cl, —Br, —I, —C D₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocyclo alkenyl group, a C₆-C₆₀ aryl group, a C₆-C₆₀ aryloxy group, a C₆-C₆₀ arylthio group, a C₁-C₆ heteroaryl group, a monovalent non-aromatic condensed polycyclic group, a monovalent non-aromatic condensed heteropolycyclic group, —Si(Q₂₁)(Q₂₂)(Q₂₃), —N(Q₂₄)(Q₂₅), or —B(Q₂₆)(Q₂₇), or
—Si(Q₃₁)(Q₃₂)(Q₃₃), —N(Q₃₄)(Q₃₅), or —B(Q₃₆)(Q₃₇), and
Q₁ to Q₇, Q₁₁ to Q₁₇, Q₂₁ to Q₂₇, and Q₃₁ to Q₃₇ are each independently hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group.

2. The heterocyclic compound of claim 1, wherein
Cz₁ is a group represented by Formulae 2A-1 to 2A-7: 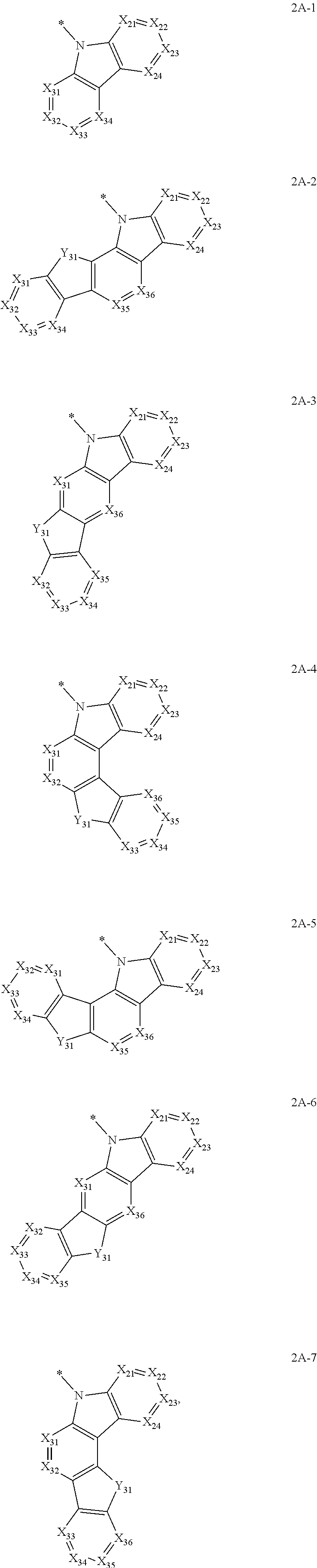
wherein, in Formulae 2A-1 to 2A-7,
X₂₁ is N or C(R₂₁), X₂₂ is N or C(R₂₂), X₂₃ is N or C(R₂₃), X₂₄ is N or C(R₂₄), and each of X₂₁ to X₂₄ is not N,
X₃₁ is N or C(R₃₁), X₃₂ is N or C(R₃₂), X₃₃ is N or C(R₃₃), X₃₄ is N or C(R₃₄), X₃₅ is N or C(R₃₅), X₃₆ is N or C(R₃₆), and each of X₃₁ to X₃₆ is not N,
Y₃₁ is O, S, N(R₃₇), C(R₃₇)(R₃₈), or Si(R₃₇)(R₃₈),
R₂₁ to R₂₄ are each independently the same as defined in connection with R₂₀ in claim 1,
R₃₁ to R₃₈ are each independently the same as defined in connection with R₃₀ in claim 1, and
* indicates a binding site to a neighboring atom.

3. The heterocyclic compound of claim 1, wherein
A₁ to A₄ are each independently a benzene group, a naphthalene group, a fluorene group, a pyridine group, a pyrazine group, a pyrimidine group, a pyridazine group, a carbazole group, a triazine group, a dibenzofuran group, a dibenzothiophene group, or a dibenzosilole group.

4. The heterocyclic compound of claim 1, wherein
A₁ to A₄ are each independently a benzene group.

5. The heterocyclic compound of claim 1, wherein
R₁ to R₁₀, R₂₀, R₃₀, R₄₀, and R₅₀ are each independently:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, or a C₁-C₂₀ alkoxy group;
a C₁-C₂₀ alkyl group or a C₁-C₂₀ alkoxy group, each substituted with at least one deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a pyridinyl group, a pyrimidinyl group, a pyrazinyl group, a pyridazinyl group, or a triazinyl group;
a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-bifluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, an imidazopyridimidinyl group, or an imidazopyridinyl group; and
a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a fluorenyl group, a spiro-bifluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, an imidazopyridimidinyl group, or an imidazopyridinyl group, each substituted with at least one deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a naphthyl group, an anthracenyl group, a pyrenyl group, a phenanthrenyl group, a fluorenyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, or a quinazolinyl group.

6. The heterocyclic compound of claim 1, wherein
R₁ to R₁₀, R₂₀, R₃₀, R₄₀, and R₅₀ are each independently:
hydrogen, deuterium, a cyano group, a C₁-C₂₀ alkyl group, or a C₁-C₂₀ alkoxy group;
a C₁-C₂₀ alkyl group or a C₁-C₂₀ alkoxy group, each substituted with at least one deuterium, a cyano group, a phenyl group, a biphenyl group, a terphenyl group, or a naphthyl group;
a cyclopentyl group, a cyclohexyl group, a cyclopentenyl group, a cyclohexenyl group, a cycloheptenyl group, a phenyl group, a biphenyl group, a terphenyl group, a naphthyl group, a fluorenyl group, a carbazolyl group, a dibenzofuranyl group, or a dibenzothiophenyl group; or
a cyclopentyl group, a cyclohexyl group, a cyclopentenyl group, a cyclohexenyl group, a cycloheptenyl group, a phenyl group, a biphenyl group, a terphenyl group, a naphthyl group, a fluorenyl group, a carbazolyl group, a dibenzofuranyl group, or a dibenzothiophenyl group, each substituted with at least one deuterium, a cyano group, a C₁-C₂₀ alkyl group, a C₁-C₂₀ alkoxy group, a phenyl group, a biphenyl group, a terphenyl group, or a naphthyl group.

7. The heterocyclic compound of claim 1, wherein
R₁ to R₁₀, R₂₀, R₃₀, R₄₀, and R₅₀ are each independently hydrogen, a cyano group, a methyl group, an ethyl group, a propyl group, an isobutyl group, a sec-butyl group, ter-butyl group, or a phenyl group.

8. The heterocyclic compound of claim 1, wherein
the heterocyclic compound does not comprise a cyano group, or
the heterocyclic compound comprises one to three cyano groups.

9. The heterocyclic compound of claim 1, wherein
the heterocyclic compound represented by Formula 1 is represented by one of Formulae 10-1 to 10-12: 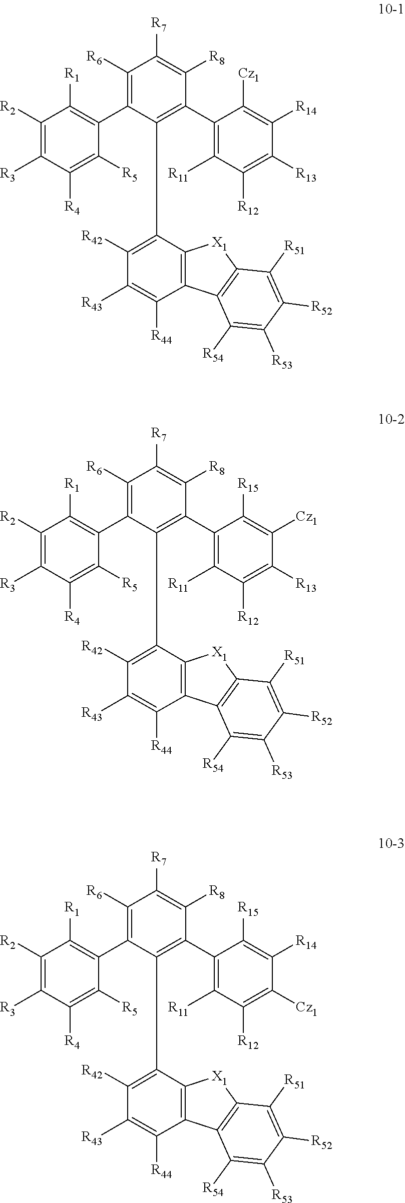 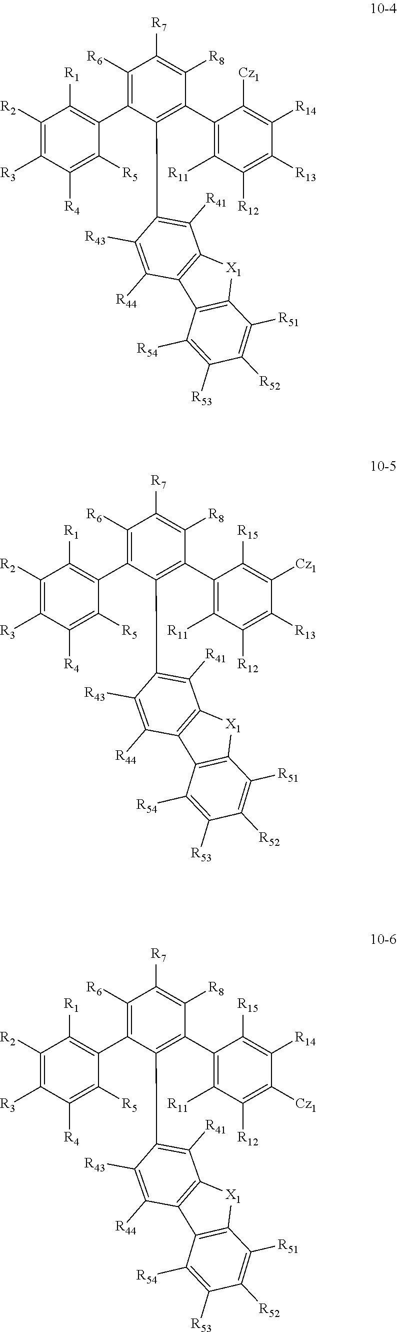 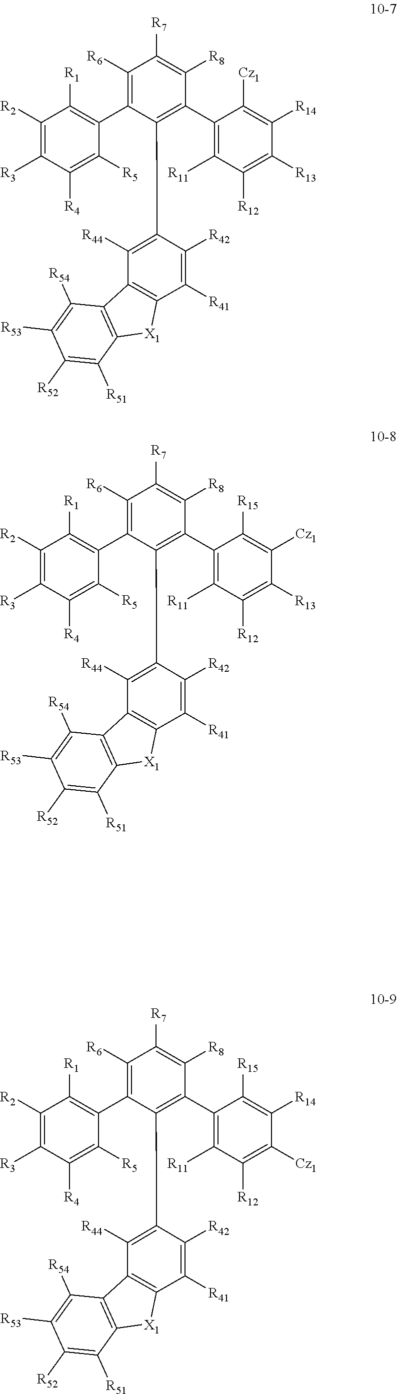 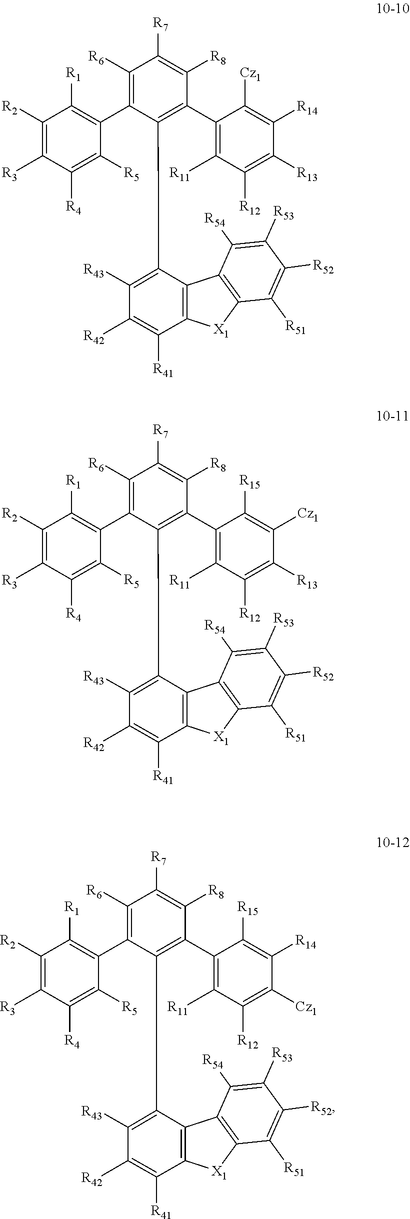
wherein, in Formulae 10-7 to 10-9,
X₁ is N(R₉),
R₁ to R₉ are each independently the same as defined in claim 1,
wherein, in Formulae 10-1 to 10-6, and 10-10 to 10-12,
X₁ is the same as defined in claim 1,
wherein, in Formulae 10-1 to 10-12,
Cz₁ is a group represented by Formula 10A: 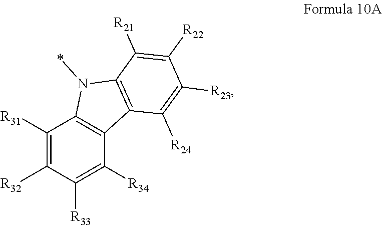
wherein, in Formulae 10-1 to 10-12 and 10A,
R₁ to R₉ are each independently the same as defined in claim 1,
R₁₁ to R₁₅ are each independently the same as defined in connection with R₁₀ in claim 1,
R₂₁ to R₂₄ are each independently the same as defined in connection with R₂₀ in claim 1,
R₃₁ to R₃₄ are each independently the same as defined in connection with R₃₀ in claim 1,
R₄₁ to R₄₄ are each independently the same as defined in connection with R₄₀ in claim 1,
R₅₁ to R₅₄ are each independently the same as defined in connection with R₅₀ in claim 1, and
* indicates a binding site to a neighboring atom.

10. The heterocyclic compound of claim 1, wherein
the heterocyclic compound has an asymmetrical structure.

11. The heterocyclic compound of claim 1, wherein
the heterocyclic compound represented by Formula 1 is one of Compounds 2 7 to 10, and 13 to 15: 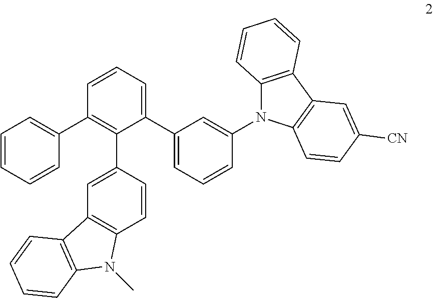 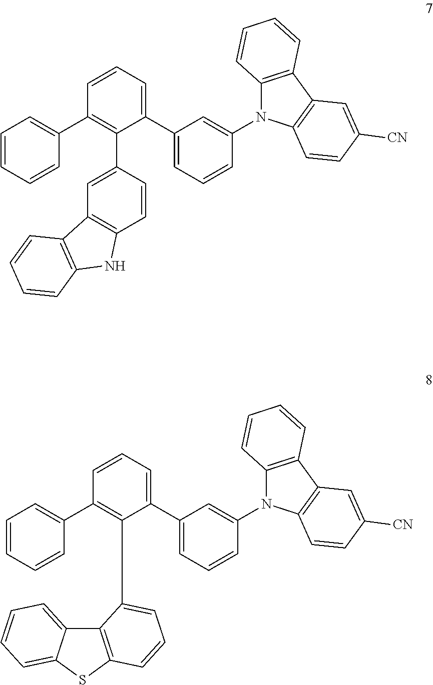 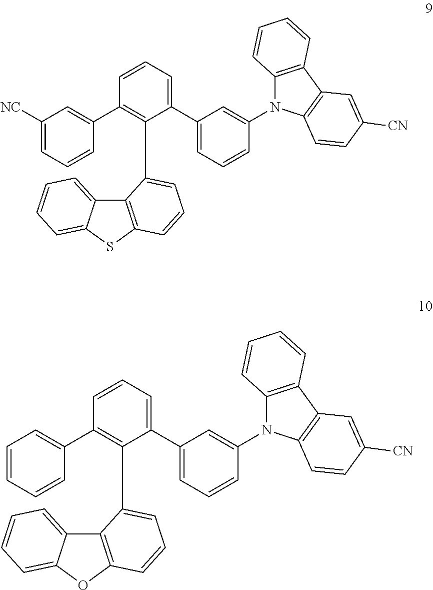 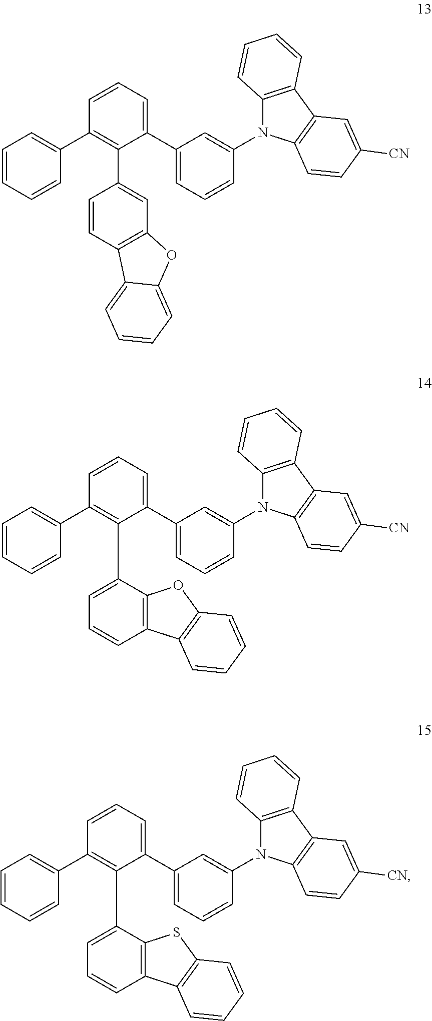
wherein, in Compounds 1 to 15, -Ph indicates a phenyl group.

12. An organic light-emitting device comprising:
a first electrode;
a second electrode; and
an organic layer between the first electrode and the second electrode and comprising an emission layer,
wherein the organic layer comprises at least one of the heterocyclic compound of claim 1 represented by Formula 1.

13. The organic light-emitting device of claim 12, wherein
the first electrode is an anode,
the second electrode is a cathode,
the organic layer further comprises a hole transport region between the first electrode and the emission layer and an electron transport region between the e mission layer and the second electrode,
the hole transport region comprises at least one of a hole injection layer, a hole transport layer, or an electron blocking layer, and
the electron transport region comprises at least one of a hole blocking layer, an electron transport layer, or an electron injection layer.

14. The organic light-emitting device of claim 13, wherein
the emission layer comprises the heterocyclic compound represented by Formula 1.

15. The organic light-emitting device of claim 14, wherein
the emission layer comprises a host and a dopant,
the host comprises the heterocyclic compound represented by Formula 1, and
an amount of the host is larger than an amount of the dopant.

16. The organic light-emitting device of claim 14, wherein
the emission layer emits blue light having a maximum emission wavelength in a range of about 410 nanometers to about 490 nanometers.

17. The organic light-emitting device of claim 13, wherein
the heterocyclic compound represented by Formula 1 is comprised in the hole transport region.

18. The organic light-emitting device of claim 13, wherein
the heterocyclic compound represented by Formula 1 is comprised in the electron transport region.
